SEQUENCE LISTING (17 sequences)

SEQ ID NO: 1 - protein, 685 aa, Homo sapiens
MDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 2 - DNA, 2759 bp, Homo sapiens
gtcactcgtataaaaacctatgctttgaaggttctcgtgtgtctcggcctgcaggtctcgctcagagctgtgtccctgaacatccaccctgctggggtggcttgacgcacttctgtgcaaatctgttcgctcgcaaccctacctacctctctcccgaaccggagaaaaccttcggcggggtccttccgggttttgtgtcgaatctgcggcggcgacccggcgccgcgtcacgcggtggtgaatgtgcggcagcacgcgcgccgcgtcgtttacgcggcgatttcatcatgctccgagccgggcggcgcgcgccgcttccgtcgccaccctctctggacagcccagggccgcaggctcatgccctctccgcgtccagtgctgcttagaggtgctcgcgccgctctgctgctgctgctgccgccccggctcttagcccgaccctcgctcctgctccgccggtccctcagcgcggcctcctgccccccgatctccttgcccgccgccgcctcccggagcagcatggacggcgcgggggctgaggaggtgctggcacctctgaggctagcagtgcgccagcagggagatcttgtgcgaaaactcaaagaagataaagcaccccaagtagacgtagacaaagcagtggctgagctcaaagcccgcaagagggttctggaagcaaaggagctggcgttacagcccaaagatgatattgtagaccgagcaaaaatggaagataccctgaagaggaggtttttctatgatcaagcttttgctatttatggaggtgttagtggtctgtatgactttgggccagttggctgtgctttgaagaacaatattattcagacctggaggcagcactttatccaagaggaacagatcctggagatcgattgcaccatgctcacccctgagccagttttaaagacctctggccatgtagacaaatttgctgacttcatggtgaaagacgtaaaaaatggagaatgttttcgtgctgaccatctattaaaagctcatttacagaaattgatgtctgataagaagtgttctgtcgaaaagaaatcagaaatggaaagtgttttggcccagcttgataactatggacagcaagaacttgcggatctttttgtgaactataatgtaaaatctcccattactggaaatgatctatcccctccagtgtcttttaacttaatgttcaagactttcattgggcctggaggaaacatgcctgggtacttgagaccagaaactgcacaggggattttcttgaatttcaaacgacttttggagttcaaccaaggaaagttgccttttgctgctgcccagattggaaattcttttagaaatgagatctcccctcgatctggactgatcagagtcagagaattcacaatggcagaaattgagcactttgtagatcccagtgagaaagaccaccccaagttccagaatgtggcagaccttcacctttatttgtattcagcaaaagcccaggtcagcggacagtccgctcggaaaatgcgcctgggagatgctgttgaacagggtgtgattaataacacagtattaggctatttcattggccgcatctacctctacctcacgaaggttggaatatctccagataaactccgcttccggcagcacatggagaatgagatggcccattatgcctgtgactgttgggatgcagaatccaaaacatcctacggttggattgagattgttggatgtgctgatcgttcctgttatgacctctcctgtcatgcacgagccaccaaagtcccacttgtagctgagaaacctctgaaagaacccaaaacagtcaatgttgttcagtttgaacccagtaagggagcaattggtaaggcatataagaaggatgcaaaactggtgatggagtatcttgccatttgtgatgagtgctacattacagaaatggagatgctgctgaatgagaaaggggaattcacaattgaaactgaagggaaaacatttcagttaacaaaagacatgatcaatgtgaagagattccagaaaacactatatgtggaagaagttgttccgaatgtaattgaaccttccttcggcctgggtaggatcatgtatacggtatttgaacatacattccatgtacgagaaggagatgaacagagaacattcttcagtttccctgctgtagttgctccattcaaatgttccgtcctcccactgagccaaaaccaggagttcatgccatttgtcaaggaattatcggaagccctgaccaggcatggagtatctcacaaagtagacgattcctctgggtcaatcggaaggcgctatgccaggactgatgagattggcgtggcttttggtgtcaccattgactttgacacagtgaacaagaccccccacactgcaactctgagggaccgtgactcaatgcggcagataagagcagagatctctgagctgcccagcatagtccaagacctagccaatggcaacatcacatgggctgatgtggaggccaggtatcctctgtttgaagggcaagagactggtaaaaaagagacaatcgaggaatgaggacaattttgacaacttttgaccacttgcgctaataaaaaaaaaaaaaaactactcttatgtccactttacaaaagaaaacagcattgtgattactcccagggaccgtattttatcttcagtggctgcctgattttacccccacaattaaagttgaaggaatcctgaacaaaaaaaaaaaa

SEQ ID NO: 3 - protein, 8 aa, Artificial Sequence
  Exemplary poly-histidine tag
LEHHHHHH

SEQ ID NO: 4 - protein, 923 aa, Homo sapiens
MERGLPLLCAVLALVLAPAGAFRNDECGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLEPILITIIAMSALGVLLGAVCGVVLYCACWHNGMSERNLSALENYNFELVDGVKLKKDKLNTQSTYSEA

SEQ ID NO: 5 - protein, 644 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSGIK

SEQ ID NO: 6 - protein, 609 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEGGTTVLATEKPTVIDSTIQSGIK

SEQ ID NO: 7 - protein, 704 aa, Homo sapiens [sequence omitted]

SEQ ID NO: 8 - protein, 644 aa, Homo sapiens; identical to SEQ ID NO: 5

SEQ ID NO: 9 - protein, 609 aa, Homo sapiens; identical to SEQ ID NO: 6

SEQ ID NO: 10 - protein, 921 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 11 - protein, 909 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFADEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 12 - protein, 901 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 13 - protein, 931 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 14 - protein, 555 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKVGCSWRPL

SEQ ID NO: 15 - protein, 906 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKGGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 16 - protein, 926 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 17 - protein, 555 aa, Homo sapiens; identical to SEQ ID NO: 14

(19) United States
(12) Patent Application Publication
(10) Pub. No.: US 2016/0144003 A1
(43) Pub. Date: May 26, 2016

(54) COMPOSITIONS AND METHODS FOR TREATING CHARCOT-MARIE-TOOTH DISEASES AND RELATED NEURONAL DISEASES

(71) Applicants: Xiang-lei Yang, San Diego, CA (US); Paul Schimmel, La Jolla, CA (US); Weiwei He, San Diego, CA (US)

(72) Inventors: Xiang-lei Yang, San Diego, CA (US); Paul Schimmel, La Jolla, CA (US); Weiwei He, San Diego, CA (US)

(73) Assignee: THE SCRIPPS RESEARCH INSTITUTE, La Jolla, CA (US)

(21) Appl. No.: 14/781,563

(22) PCT Filed: May 17, 2012
(86) PCT No.: PCT/US12/38397

§ 371 Date: Feb. 11, 2016

Related U.S. Application Data
(60) Provisional application No. 61/487,900, filed on May 19, 2011.
(60) Provisional application No. 61/572,619, filed on Jul. 19, 2011.

(51) Int. Cl. A61K 38/53

(52) U.S. Cl. CPC A61K 38/53

(57) ABSTRACT

Provided are compositions and methods for the treatment of mutant glycyl-tRNA synthetase (GlyRS)-associated diseases, such as Charcot-Marie-Tooth (CMT) diseases, and related compositions and methods for diagnostic, drug discovery, and research applications. Also provided are mutant glycyl-tRNA synthetases and uses thereof.

### CROSS REFERENCE TO RELATED APPLICATIONS

This application claims the benefit under 35 U.S.C. §119(e) of U.S. Provisional Application No. 61/572,619, filed Jul. 19, 2011; and U.S. Provisional Application No. 61/487,900, filed May 19, 2011, each of which is incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ATYR_105_01 WO_ST25txt. The text file is about 97 KB, was created on May 18, 2012, and is being submitted electronically via EFS-web, concurrent with the filing of the specification.

### TECHNICAL FIELD

The present invention relates to the discovery of neomorphic regions of glycyl-tRNA synthetase (GlyRS) associated with certain diseases such as Charcot-Marie-Tooth (CMT) diseases, the interaction of these GlyRS neomorphic regions with neuropilin transmembrane receptors, and related compositions and methods for diagnostic, drug discovery, research, and therapeutic applications.

### BACKGROUND

Charcot-Marie-Tooth (CMT) disease, also known as hereditary motor and sensory neuropathy, was named after three physicians who first described the disease in 1886. The disease is characterized by loss of muscle tissue and touch sensation in body extremities, predominantly in the feet and legs but also in the hands and arms (See Patzko A, Shy M E, Curr Neurol Neurosci Rep 11:78-88, 2011). Presently incurable, this disease is one of the most common inherited neurological disorders affecting 1 in 2,500 people (See Skre H, Clin Genet 6:98-118, 1974). Genetically, CMT disease is a heterogeneous group of disorders (See Barisic N, et al., Ann Hum Genet 72:416-441, 2088). Among the forty or so genes identified so far whose mutations are linked to the similar clinical presentations of CMT, four are genes encoding aminoacyl-tRNA synthetase, namely glycyl, tyrosyl, alanyl, and lysyl tRNA synthetases (See Antonellis et al., Am J Hum Genet 72:1293-1299, 2003; Jordanova, et al., Nat Genet 38:197-202, 2006; Latour, et al., Am J Hum Genet 86:77-82, 2010; and McLaughlin, et al., Am J Hum Genet 87:560-566, 2010).

Aminoacyl-tRNA synthetases are a family of essential enzymes in translation (See Ling et al., Annu Rev Microbial 63:61-78, 2009). Each member is responsible for charging one specific amino acid onto its cognate tRNAs. The charged tRNAs then use the embedded 3-nucleotide anticodons to decode mRNA and provide the corresponding amino acid building blocks for protein synthesis on the ribosome. Encoding glycyl-tRNA synthetase (GlyRS), GARS was the first tRNA synthetase gene implicated in CMT (See Antonellis et al., supra). So far, at least eleven different missense mutations of GARS have been reported to cause a dominant axonal form of CMT (e.g., CMT type 2D) in patients (See Antonellis et al., supra; James, et al., Neurology 67:1710-1712, 2006; Abe and Hayasaka, J Hum Genet 54:310-312, 2009; and Motley et al., Trends Neurosci 33:59-66, 2010). Two separate spontaneous or ENU-induced missense mutations have also been linked to CMT-like phenotypes in mice (See Seburn et al., Neuron 51:715-726, 2006; and Achilli, et al., Dis Model Mech 2:359-373, 2009). However, not all mutations affect the aminoacylation activity of the tRNA synthetase (See Nangle et al., Proc Natl Acad Sci USA 104:11239-11244, 2007; and Antonellis et al., J Neurosci 26:10397-10406, 2006). Furthermore, studies in mice demonstrated that the CMT-like phenotype was not caused by haplo-insufficiency in protein synthesis, but rather by a pathogenic role of the mutant GlyRS itself (See Sebum et al., supra; and Stum M, et al., Mol Cell Neurosci 46:432-443, 2011), which remains to be defined at the molecular level.

GlyRS is a class II tRNA synthetase, whose catalytic domain consists of a central anti-parallel β sheet flanked with a helices, and three conserved sequence motifs (motifs 1-3) (See Xie et al., PNAS USA 104:9976-9981, 2007. Human GlyRS has three insertions that split the catalytic domain, a metazoan-specific helix-turn-helix WHEP domain, and an anticodon binding domain at the N- and C-terminal side of the catalytic domain, respectively. Like most class II tRNA synthetases, GlyRS functions as a dimer for aminoacylation. Despite being well-separated in the primary sequence of the three domains of GlyRS, all known CMT-causing mutations are located near the dimer interface of our crystal structure (See Nangle et al., supra). However, different CMT-causing mutations have different effects on dimer formation; for instance, some disrupt, some strengthen, and some seem to have no effect on the dimer (See Id.). In addition, crystal structures of two CMT-causing mutant proteins showed little difference from that of the WI protein (See Xie et al., supra; and Cader, et al., FEBS Lea 581:2959-2964, 2007), suggesting that structural differences, if any, between mutant and WT GlyRSs are subtle and might be suppressed by crystal packing forces.

Closer exploration of mutant GlyRS structures at the molecular level could thus lead to identification and characterization of GlyRS structures associated with various diseases such as CMT diseases.

### BRIEF DESCRIPTION OF THE DRAWINGS

FIG. 1 shows the distribution of CMT-causing mutations on GlyRS. In FIG. 1A, all 13 CMT-causing mutations are mapped onto the domain structure of GlyRS. The three sequence motifs that are characteristic of the catalytic domain of class II tRNA synthetases are noted as 1, 2, 3, and the three insertions to the catalytic domain as I, II, III. FIG. 1B shows the dimer interface location of two newly identified CMT-associated residues C157 and P244. FIG. 1C shows a close-up view of the location of C157 and P244.

FIGS. 2A and 2B illustrate the changes in deuterium incorporation resulting from CMT-causing mutations or deletion of the WHEP domain. The results are mapped on the primary sequence of GlyRS, with CMT mutation-associated residues highlighted in purple (as also shown in FIG. 1A). The percent difference of deuterium incorporation was calculated from the hydrogen-deuterium exchange after 1 hour for each mutant relative to WT GlyRS. The uncovered areas for each mutant may result from either lack of sequence coverage for either the mutant or WT GlyRS, or lack of common peptides for direct comparison. The sequence coverage for WT, L129P, G240R, G526R, S581L, G598A and ΔWHEP GlyRS was 96%, 87%, 95%, 89%, 98%, 99% and 96%, respectively. The eight consensus opened-up areas (hot spots) are labeled. In general, the hot spots areas are well-covered in at least 4 of the 5 CMT mutants tested, and each mutant peptide within the hot spots showed more than 5% increase in HDX relative to WT.

FIG. 3 shows the changes in deuterium incorporation mapped onto the crystal structure of GlyRS. FIGS. 3A-3F show mapping of changes in deuterium incorporation caused by different CMT mutations or deletion of the WHEP domain, as indicated. The monomeric structure is oriented to view the dimerization interface. The color coding/shading is the same as in FIG. 2. FIG. 3G shows mapping of the consensus areas (or hot spots) that are opened-up by all 5 tested CMT-causing mutations. The hot spots are colored in gold (medium gray) and CMT mutation-associated residues in purple (darkest gray).

In FIG. 4, SAXS analysis confirms the structure opening of G526R GlyRS and also reveals the conformational change of the WHEP domain. FIG. 4A shows solution scattering data of WT and G526R GlyRS. Small-angle X-ray scattering curves are overlapped with theoretical scattering profiles calculated from ab initio models (black line). The inset shows Guinier plots at the low-angle region (S*R_{g}<1.3). FIG. 4B shows distance distribution P(R) functions of WT and G526R GlyRS. P(R) curves were calculated from SAXS data shown in FIG. 4A. The main differences between WT and G526R GlyRS are indicated by arrows. FIGS. 4C and 4D show SAXS-based ab initio modeling of WT (purple; dark grey) and G526R (yellow; light grey) GlyRS. The crystal structure of the dimeric WT GlyRS was manually docked into the SAXS-based molecular envelop, leaving two extra densities located on each side of the dimer near the N-terminus of the GlyRS structure. Therefore, the extra densities most likely correspond to the disordered WHEP domain in the crystal structure. The extra densities are fit with a model of the WHEP domain from HisRS (PDB:1X59) and differ between WT and G526R GlyRS, suggesting a conformational change of the WHEP domain induced by the G526R mutation.

FIG. 5 shows in illustration of the same conformational opening of GlyRS induced by different CMT-causing mutations and of the generation of a common neomorphic surface.

FIGS. 6A-6F show deuterium uptake time-course curves for representative peptides in CMT-causing mutants compared with those in WT GlyRS. (FIG. 6A) L129P. (FIG. 6B) G240R. (FIG. 6C) G526R. (FIG. 6D) S581L. (FIG. 6E) G598A. (FIG. 6F) WHEP domain deletion. Blue or darker line is WT GlyRS, and red or lighter line is the mutant.

FIG. 7 shows the level of deuterium incorporation for WT GlyRS after 1 hour hydrogen-deuterium exchange. The percentage incorporation is the observed number of incorporated deuterium isotopes to the total number of exchangeable amide protons in a peptide. FIG. 7A shows the result mapped onto the primary sequence of GlyRS. Here, peptides from the disordered region in the crystal structure of GlyRS (e.g., the WHEP domain, Insertion III and the C-terminus) generally have high levels of deuterium uptake. FIG. 7B shows dimeric (stereo) and monomeric (mono) views of the GlyRS crystal structure with specific domains, motifs and insertions labeled. FIG. 7C shows HDX results mapped onto the crystal structure of GlyRS; peptides with high deuterium uptake are located on the surface of the dimeric crystal structure.

FIGS. 8A-8F show sedimentation equilibrium analysis of WT and CMT-causing mutant human GlyRS proteins. Three cells containing different concentrations of each protein sample were centrifuged at 9000 rpm (solid curved line), 14000 rpm (mixed-dash curved line), and 19000 rpm (dash curved line). Absorbance data for each cell was collected at 280 nm and 230 nm wavelength. The meniscus (vertical dot-dash line) in each cell was set manually, while the bottom (vertical dash line) was allowed to float. Data within the fit limits (vertical solid lines) was globally fitted to the monomer-dimer self-association model in SEDPHAT. Data beyond approximately 1.5 absorbance was omitted in case of non-linearity of the detection instrumentation. The fits for the 9000, 14000, and 19000 rpm data are represented by the solid, mixed dash, and dash lines respectively. The residuals for each fit are displayed in the lower panel of each plot with the same color scheme.

FIG. 9A shows the detection of GlyRS in human and mouse serum by SDS-PAGE. The level of GlyRS in serum, was detected by anti-GlyRS (anti-V5) antibody and cell necrosis was monitored by tubulin, GAPDH and LDH. N2a cell lysate was used as positive control for different antibodies. The SDS-PAGE analysis in FIG. 9B shows that the WHEP domain of GlyRS is required for secretion of GlyRS. Full-length cytoplasmic GlyRS can be detected in both whole cell lysate and cell medium. The ΔWHEP mutant can be detected in whole cell lysate but not cell medium.

FIGS. 10A-10B illustrate the results of an in vitro pull-down assay, where CMT-associated mutants of GlyRS show enhanced interaction with neuropilin. FIG. 10A shows the reaction scheme, and FIG. 10B shows the results of western-blotting with anti-His antibody (Lane 1 is wild-type GlyRS; Lane 2 is L129P mutant; Lane 3 is G240R mutant; and Lane 4 is G526R mutant).

FIGS. 11A-11B illustrate the results of a cell-surface binding experiment in Hela cells overexpressing a GFP-neuropilin fusion protein, where CMT-associated mutants of GlyRS show enhanced interaction with neuropilin. FIG. 11A shows the experimental scheme, and FIG. 11B shows the results of western-blotting to detect His-GRS and GFP-neuropilin (NRP1) interactions (Lanes 1 and 2 are wild-type GlyRS; Lane 3 is L129P mutant; Lane 4 is G240R mutant; Lane 5 is S581L mutant; and Lane 6 is ΔWHEP mutant).

FIGS. 12A-12G show the effect of GlyRS mutants on neuropilin-induced neurite outgrowth in N2a cells. FIG. 12A shows GFP staining of N2a cells transfected with GFP alone, compared to FIG. 12B, which shows GFP staining of N2a cells transfected with GFP-NRP1. Increased neurite outgrowth can be observed in FIG. 12B, relative to FIG. 12A. FIG. 12C and FIG. 12D show that incubation with wild-type GlyRS had no effect on neuropilin-induced neurite outgrowth. FIG. 12C shows filopedia and FIG. 12D shows lamellipodia of GFP-NRP1 expressing N2a cells incubated with wild-type GlyRS. FIGS. 12E (L129P mutant), 12F (G240R mutant), and 12G (G526R mutant) show that incubation with CMT-associated GlyRS mutants reverses neuropilin-induced neurite outgrowth. These images also show a stronger interaction between the CMT mutants and the neuropilin-transfected cells relative to wild-type GlyRS.

FIG. 13 shows a quantitative analysis of NRP1 and GlyRS binding, based on cell counts of confocal microscopy images of GlyRS (wild-type or mutants) and GFP-NRP1-expressing cells relative to GlyRS (wild-type or mutant) and GFP-only expressing cells.

FIGS. 14A-14C show the results of a competition binding assay of GlyRS to neuropilin. FIG. 14A illustrates the experimental scheme, and FIGS. 14B-14C show the results of western-blotting to detect interaction of His-GlyRS or V5-sema3A with neuropilin (NRP1). FIG. 14B shows the results for wild-type GlyRS, which does not competitively inhibit binding between sema3A and neuropilin, and FIG. 14C shows the results for the L129P mutant, which does competitively inhibit binding between sema3A and neuropilin.

FIGS. 15A-15C show the binding affinity between various GlyRS polypeptides and neuropilin-1 (NRP-1). In FIG. 15A, the binding between NRP-1 and GlyRS (wild-type (WT), CMT-associated mutant L129P, and CMT-associated mutant P234KY) at 1 μM was compared by bio-layer interferometry in Octet RED system. No binding was detected between NRP-1 and WT GlyRS, but strong binding was detected between NRP-1 and each of the CMT-associated mutants L129P and P234KY. FIG. 15B shows the kinetic analysis of the binding between L129P and NRP-1 at five concentrations listed. Global fitting of the binding data for the five concentrations of L129P resulted in a dissociation constant of 36.8 nM. FIG. 15C shows the kinetic analysis of P234KY binding to NRP1 at three concentrations listed. Global fitting of binding data for the three concentrations of P234KY resulted in a dissociation constant of 162 nM.

### BRIEF SUMMARY OF THE INVENTION

Embodiments of the present invention relate to the discovery of neomorphic regions of glycyl-tRNA synthetase (GlyRS), which show increased solvent exposure in disease-associated and other mutant forms of GlyRS relative to wild-type GlyRS, and which may thus play a role in one or more non-canonical activities of disease-associated GlyRS mutants. Many of these solvent-exposed or opened-up surfaces are shared by a number of disease-associated GlyRS mutants, suggesting their utility as therapeutic targets for antibodies, binding agents, or small molecules in the treatment of a variety of diseases, such as Charcot-Marie-Tooth diseases, and other GlyRS-associated diseases. Polypeptides or other molecules comprising these neomorphic regions could also be useful in drug discovery or other basic science applications, for example, to identify antibodies, binding agents, or small molecules having binding specificity for one or more neomorphic regions of GlyRS, and to identify cellular binding partners of disease-associated GlyRS mutants.

It has also been unexpectedly discovered that certain disease-associated GlyRS mutants, including CMT-associated GlyRS mutants, specifically interact with neuropilin(s), including for example the neuropilin transmembrane co-receptors (e.g., neuropilin-1 or NRP-1). Because neuropilins play a general role in axon guidance during the development of the nervous system in vertebrates, and play other important roles in normal physiology, this discovery suggests that disease-associated GlyRS mutants may mediate disease progression at least in part through the negative regulation of neuropilins. This discovery thus enables the development of specific screening assays to identify molecules such as antibodies or other binding agents that can block the interaction between disease-associated mutant GlyRS and neuropilins. It also suggests that soluble isoforms of neuropilins could sequester disease-associated GlyRS mutants, and thereby reduce the symptoms or progression of GlyRS-associated diseases, such as CMT and other diseases mediated by GlyRS mutants.

Additionally, because neuropilins play diverse roles during the physiological regulation of processes such as angiogenesis, axon guidance, cell survival, migration, and invasion, the ability of GlyRS mutants to specifically interact with neuropilins further suggests that these GlyRS mutants (or other molecules with exposed neomorphic regions of GlyRS) may have therapeutic utility in their own right, for example, where physiological problems occur due to aberrant activity of neuropilins, aberrant activity of neuropilin ligands such as vascular endothelial growth factor (VEGF), placental growth factors (PGFs), heparin-binding proteins, fibroblast growth factor-2 (FGF-2), hepatocyte growth factor (HGF), and semaphorin family members (e.g., Sema3A), and/or aberrant activity of other members of neuropilin-related pathways described herein and known in the art.

Accordingly, embodiments of the present invention include an antibody or antigen-binding fragment thereof that exhibits binding specificity for at least one neomorphic region of human glycyl-tRNA synthetase (GlyRS), wherein said at least one neomorphic region is located within a region defined by amino acid residues A57 to A663 of full-length human GlyRS (SEQ ID NO:1), or an antigenic fragment of said region. In certain embodiments, the neomorphic region is selected from one or more regions defined by amino acid residues A57-A83, L129-D161, N208-Y320, V366-H378, P518-M531, L584-Y604, F620-R635, and D654-A663 of full-length human GlyRS (SEQ ID NO:1), or a combination or an antigenic fragment of said region(s). In some embodiments, the antibody or antigen binding fragment binds to both wild type human GlyRS and a mutant human GlyRS; wherein said mutant GlyRS comprises at least one disease associated mutation. In certain embodiments, affinity of said antibody or antigen-binding fragment thereof for said mutant human GlyRS is stronger than its affinity for wild type human GlyRS by at least about 2× to at least about 100× or to at least about 1000×.

In some embodiments the antibody or antigen binding fragment binds specifically to a mutant GlyRS, but does not substantially bind to the wild type full length GlyRS, wherein the mutant GlyRS comprises at least one disease associated mutation. In some aspects, binding of the antibody or antigen binding fragment blocks or reduces the binding of the mutant GlyRS to a neuropilin(s).

In particular embodiments, the disease associated mutation is associated with Charcot-Marie-Tooth Disease Type 2D or Distal Spinal Muscular Atrophy Type V (dSMA-V) disease. In specific embodiments, the disease associated mutation is associated with Charcot-Marie-Tooth Disease Type 2D. In certain embodiments, the disease associated mutation is A57V, E71G, L129P, C157R, P234KY, G240R, P244L, I280F, H418R, D500N, G526R, S581L, or G598A. In certain embodiments, the disease associated mutation is selected from the group consisting of L129P, G240R, G526R, S581L, and G598A.

In some embodiments, the disease associated mutation is L129P. In particular embodiments, said neomorphic region is selected from one or more regions defined by amino acid residues A57-A83, G97-T110, E119-S178, N208-Y320, A326-N348, L361-H378, K423-E429, V461-Y464, L480-F486, K505-P554, V564-N570, L584-Y604, F620-I645, or D654-A663 of full-length human GlyRS (SEQ ID NO:1), or an antigenic fragment of said region(s), and wherein affinity of the antibody for the L129P mutant of human GlyRS is stronger than its affinity for wild type human GlyRS by at least about 2× to at least about 100× or to at least about 1000×.

In certain embodiments, the disease associated mutation is G240R. In specific embodiments, said neomorphic region is selected from one or more regions defined by amino acid residues A57-A83, G97-E123, F147-L189, F204-Y320, N348-H378, V461-Y464, K483-M531, D545-R642, or D654-E685 of full-length human GlyRS (SEQ ID NO:1), or an antigenic fragment of said region(s), wherein affinity of the antibody for the G240R mutant of human GlyRS is stronger than its affinity for wild type human GlyRS by at least about 2× to at least about 100× or to at least about 1000×.

In certain embodiments, said disease associated mutation is G526R. In specific embodiments, said neomorphic region is selected from one or more regions defined by amino acid residues A57-A83, L129-K150, S183-V188, N208-Y320, N348-D389, K423-E429, L480-E485, D500-L511, P518-M531, T538-F550, L584-Y604, F620-I645, or D654-A663 of full-length human GlyRS (SEQ ID NO:1), or an antigenic fragment of said region(s), and wherein affinity of the antibody for the G526R mutant of human GlyRS is stronger than its affinity for wild type human GlyRS by at least about 2× to at least about 100× or to at least about 1000×.

In some embodiments, said disease associated mutation is S581L. In particular embodiments, said neomorphic region is selected from one or more regions defined by amino acid residues A57-107, L129-D161, N208-Y320, V366-I402, K493-Q496, V513-M531, A555-R635, or D654-E685 of full-length human GlyRS (SEQ ID NO:1), or an antigenic fragment of said region(s), and wherein affinity of the antibody for the S581L mutant of human GlyRS is stronger than its affinity for wild type human GlyRS by at least about 2× to at least about 100× or to at least about 1000×.

In certain embodiments, said disease associated mutation is G598A. In some embodiments, said neomorphic region is selected from one or more regions defined by amino acid residues A57-N106, L129-L203, N208-Y320, V366-D389, A421-Y464, E504-M531, F551-I645, or D654-A663, or an antigenic fragment of said region(s), and wherein affinity of the antibody for the G598A mutant of human GlyRS is stronger than its affinity for wild type human GlyRS by at least about 2× to at least about 100× or to at least about 1000×.

In certain embodiments, said disease is Distal Spinal Muscular Atrophy Type V (dSMA-V). In specific embodiments, said disease associated mutation is E71G, L129P, P234KY, G240R or I280F.

Certain embodiments include an antibody or antigen-binding fragment that fully or partially antagonizes an interaction between a human GlyRS mutant associated with a disease and its cellular binding partner(s). In specific embodiments, the cellular binding partner is a neuropilin, such as, for example, the neuropilin transmembrane receptor, such as neuropilin-1 (NRP1). Some embodiments include an antibody or antigen-binding fragment which modulates the rate of deuterium uptake of a human GlyRS mutant associated with a disease. Certain embodiments include an antibody or antigen-binding fragment which exhibits bind specificity for the monomer but not the dimer of human wild type GlyRS.

Also included are binding agents or small molecules that exhibit binding specificity for at least one neomorphic region of human glycyl-tRNA synthetase (GlyRS), wherein said at least one neomorphic region is located within a region defined by amino acid residues A57 to A663 of full-length human GlyRS (SEQ ID NO:1), or an antigenic fragment of said region. In some embodiments, said neomorphic region is selected from one or more regions defined by amino acid residues A57-A83, L129-D161, N208-Y320, V366-H378, P518-M531, L584-Y604, F620-R635, and D654-A663 of full-length human GlyRS (SEQ ID NO:1), or a combination or an antigenic fragment of said region(s). In some embodiments, said binding agent or small molecule binds to both wild type human GlyRS and a mutant GlyRS; wherein said mutant GlyRS comprises at least one disease associated mutation. In particular embodiments, affinity of said binding agent or small molecule for said mutant human GlyRS is stronger than its affinity for wild type human GlyRS by at least about 2× to at least about 100× or to at least about 1000×.

In some embodiments the binding agents or small molecules bind specifically to a mutant GlyRS, but does not substantially bind to the wild type full length GlyRS, wherein the mutant GlyRS comprises at least one disease associated mutation. In some aspects, binding of the binding agents or small molecules block the binding of the mutant GlyRS to a neuropilin(s).

In certain embodiments, the disease associated mutation is associated with Charcot-Marie-Tooth Disease Type 2D or Distal Spinal Muscular Atrophy Type V (dSMA-V) disease. In some embodiments, the disease associated mutation is associated with Charcot-Marie-Tooth Disease Type 2D. In specific embodiments, the disease associated mutation is A57V, E71G, L129P, C157R, P234KY, G240R, P244L, I280F, H418R, D500N, G526R, S581L, or G598A. In certain embodiments, the disease associated mutation is selected from the group consisting of L129P, G240R, G526R, S581L, and G598A.

In certain embodiments, said disease associated mutation is L129P. In specific embodiments, said neomorphic region is selected from one or more regions defined by amino acid residues A57-A83, G97-T110, E119-S178, N208-Y320, A326-N348, L361-H378, K423-E429, V461-Y464, L480-F486, K505-P554, V564-N570, L584-Y604, F620-I645, or D654-A663 of full-length human GlyRS (SEQ ID NO:1), or an antigenic fragment of said region(s), and wherein affinity of the binding agent for the L129P mutant of human GlyRS is stronger than its affinity for wild type human GlyRS by at least about 2× to at least about 100× or to at least about 1000×.

In certain embodiments, said disease associated mutation is G240R. In particular embodiments, said neomorphic region is selected from one or more regions defined by amino acid residues A57-A83, G97-E123, F147-L189, F204-Y320, N348-H378, V461-Y464, K483-M531, D545-R642, or D654-E685 of full-length human GlyRS (SEQ ID NO:1), or an antigenic fragment of said region(s), and wherein affinity of the binding agent or small molecule for the G240R mutant of human GlyRS is stronger than its affinity for wild type human GlyRS by at least about 2× to at least about 100× or to at least about 1000×.

In certain embodiments, said disease associated mutation is G526R. In some embodiments, said neomorphic region is selected from one or more regions defined by amino acid residues A57-A83, L129-K150, S183-V188, N208-Y320, N348-D389, K423-E429, L480-E485, D500-L511, P518-M531, T538-F550, L584-Y604, F620-I645, or D654-A663 of full-length human GlyRS (SEQ ID NO:1), or an antigenic fragment of said region(s), and wherein affinity of the binding agent or small molecule for the G526R mutant of human GlyRS is stronger than its affinity for wild type human GlyRS by at least about 2× to at least about 100× or to at least about 1000×.

In certain embodiments, said disease associated mutation is S581L. In some embodiments, said neomorphic region is selected from one or more regions defined by amino acid residues A57-107, L129-D161, N208-Y320, V366-I402, K493-Q496, V513-M531, A555-R635, or D654-E685 of full-length human GlyRS (SEQ ID NO:1), or an antigenic fragment of said region(s), and wherein affinity of the binding agent or small molecule for the S581L mutant of human GlyRS is stronger than its affinity for wild type human GlyRS by at least about 2× to at least about 100× or to at least about 1000×.

In particular embodiments, said disease associated mutation is G598A. In certain embodiments, said neomorphic region is selected from one or more regions defined by amino acid residues A57-N106, L129-L203, N208-Y320, V366-D389, A421-Y464, E504-M531, F551-I645, or D654-A663 of full-length human GlyRS (SEQ ID NO:1), or an antigenic fragment of said region(s), and wherein affinity of the binding agent or small molecule for the G598A mutant of human GlyRS is stronger than its affinity for wild type human GlyRS by at least about 2× to at least about 100× or to at least about 1000×.

In certain embodiments, the disease is Distal Spinal Muscular Atrophy Type V (dSMA-V). In specific embodiments, the disease associated mutation is E71G, L129P, P234KY, G240R or I280F.

Certain embodiments include a binding agent or small molecule which fully or partially antagonizes an interaction between a human GlyRS mutant associated with a disease and its cellular binding partner(s). In specific embodiments, the cellular binding partner is a neuropilin, such as, for example, a neuropilin transmembrane receptor, such as neuropilin-1 (NRP1). Some embodiments include a binding agent or small molecule which modulates the rate of deuterium uptake of a human GlyRS mutant associated with a disease. Particular embodiments include a binding agent or small molecule which exhibits bind specificity for the monomer but not the dimer of human wild type GlyRS. Also included are binding agents selected from adnectins, anticalins, avimers, DARPins, and aptamers. Certain binding agents include one or more soluble isoforms of a neuropilin transmembrane receptor, such as a soluble isoform of NRP1.

Some embodiments include compositions, comprising an antibody or antigen-binding fragment described herein, a binding agent or small molecule described herein, or both, and a pharmaceutically acceptable carrier. In specific embodiments, the composition is for treating Charcot-Marie-Tooth Disease Type 2D or Distal Spinal Muscular Atrophy Type V (dSMA-V).

Also included are methods of reducing or ameliorating a symptom of Charcot-Marie-Tooth Disease Type 2D or Distal Spinal Muscular Atrophy Type V (dSMA-V) disease, comprising administering to a subject an antibody or antigen-binding fragment described herein, a binding agent or small molecule described herein, or a composition described herein, where said subject has a glycyl-tRNA synthetase (GlyRS)-disease associated mutation. In particular embodiments, said disease associated mutation is A57V, E71G, L129P, C157R, P234KY, G240R, P244L, I280F, H418R, D500N, G526R, S581L, or G598A. In certain embodiments, the disease associated mutation is selected from the group consisting of L129P, G240R, G526R, S581L, and G598A.

Also included are methods for identifying an antibody or antigen-binding fragment thereof, or a binding agent or small molecule, which modulates a disease mediated by a human mutant tRNA synthetase, comprising a) incubating said antibody or antigen-binding fragment thereof, or binding agent or small molecule, with said mutant tRNA synthetase, and b) measuring the rate of deuterium exchange in the presence of said antibody or antigen-binding fragment thereof, or binding agent or small molecule, relative to the rate of deuterium exchange in the absence of said antibody or antigen-binding fragment thereof, or binding agent or small molecule, wherein a difference in the rates of deuterium exchange in the presence and absence of said antibody or antigen-binding fragment thereof, or binding agent or small molecule; indicates that said antibody or antigen-binding fragment thereof, or binding agent or small molecule, is capable of modulating a disease mediated by said human mutant tRNA synthetase. In some embodiments, said mutant tRNA synthetase is a GlyRS, a TyrRS, an AlaRS, or a LysRS.

Also included are methods of identifying cellular binding partner of a human glycyl-tRNA synthetase (GlyRS) mutant associated with a Charcot-Marie-Tooth (CMT) disease, comprising a) combining a polypeptide that comprises an exposed neomorphic region of human GlyRS, where said neomorphic region is one or more of A57-A663, A57-A83, L129-D161, N208-Y320, V366-H378, P518-M531, L584-Y604, F620-R635, or D654-A663, or an antigenic fragment of said region(s), with one or more candidate cellular binding partner(s), and b) detecting or determining binding of said neomorphic region of said polypeptide to one or more of said candidate cellular binding partner(s), thereby identifying a cellular binding partner that specifically binds to a neomorphic region of human GlyRS. In certain embodiments, said polypeptide is a fusion protein that comprises at least one heterologous sequence and one or more of said neomorphic regions. In certain embodiments, said polypeptide consists essentially of A57-A663, A57-A83, L129-D161, N208-Y320, V366-H378, P518-M531, L584-Y604, F620-R635, or D654-A663 of human GlyRS, or an antigenic fragment or combination thereof. In some embodiments, one or more of said candidate cellular binding partner(s) is a cell-surface receptor or an extracellular portion thereof.

Also included are methods of diagnosing a Charcot-Marie-Tooth (CMT) disease in a subject, comprising contacting a biological sample from said subject with an antibody or antigen-binding fragment described herein, or a binding agent or small molecule described herein, where detecting a specific interaction between said antibody or antigen-binding fragment, or said binding agent or small molecule, and a glycyl-tRNA synthetase (GlyRS) in said sample indicates that said subject has a CMT disease.

Also included are methods of identifying a subject with Charcot-Marie-Tooth (CMT) disease who would benefit from treatment with an antibody or antigen-binding fragment described herein, or a binding agent or small molecule described herein, or a neuropilin polypeptide as described herein, comprising contacting a biological sample from said subject with said antibody or antigen-binding fragment described herein, or said binding agent or small molecule described herein or said neuropilin polypeptide, where detecting a specific interaction between said antibody or antigen-binding fragment, or said binding agent or small molecule, or neuropilin polypeptide and a mutant glycyl-tRNA synthetase (GlyRS) in said sample indicates that said subject would benefit from therapy with an antibody or antigen-binding fragment described herein, or a binding agent or small molecule described herein or a neuropilin polypeptide as described herein.

Certain embodiments include isolated peptides, consisting essentially of residues A57-A663 of wild-type GlyRS (SEQ ID NO:1), or an antigenic fragment thereof. Also included are isolated polypeptides, consisting essentially of residues A57-A83, L129-D161, N208-Y320, V366-H378, P518-M531, L584-Y604, F620-R635, or D654-A663 of wild-type GlyRS, or an antigenic fragment thereof. Certain embodiments include isolated peptides, consisting essentially of residues F79-A83, M227-L257, I232-N253, L258-E279, F147-K150, or E515-M531 of wild-type GlyRS. Specific embodiments include isolated peptides, consisting of residues F79-A83, M227-L257, I232-N253, L258-E279, F147-K150, or E515-M531 of wild-type GlyRS. Some embodiments include isolated peptides, consisting of residues A57-A663, A57-A83, L129-D161, N208-Y320, V366-H378, P518-M531, L584-Y604, F620-R635, or D654-A663 of wild-type GlyRS. In certain embodiments, the isolated peptide specifically binds to a neuropilin.

Also included are fusion proteins, comprising any one or more of the mutant GlyRS peptides described herein and a heterologous fusion partner. Certain embodiments include isolated polynucleotides, which encode a peptide or fusion protein described herein.

Also included are methods of identifying an antibody, binding agent or small molecule that exhibits binding specificity for a neomorphic region of human glycyl-tRNA synthetase (GlyRS), comprising a) combining a polypeptide that comprises an exposed neomorphic region of human GlyRS, where said neomorphic region is one or more of A57-A663, A57-A83, L129-D161, N208-Y320, V366-H378, P518-M531, L584-Y604, F620-R635, or D654-A663, or a combination or an antigenic fragment of said region(s), with at least one test antibody, binding agent or small molecule under suitable conditions, and b) detecting or determining binding of the neomorphic region of the polypeptide to the test antibody, binding agent or small molecule, thereby identifying an antibody, binding agent or small molecule that specifically binds to a neomorphic region of human GlyRS.

Also included are processes for manufacturing a pharmaceutical composition, wherein said composition comprises an antibody, binding agent or small molecule, comprising: a) performing an in vitro screen of one or more test antibodies, binding agents or small molecules in the presence of a polypeptide that comprises an exposed neomorphic region of human GlyRS, where said neomorphic region is one or more of A57-A663, A57-A83, L129-D161, N208-Y320, V366-H378, P518-M531, L584-Y604, F620-R635, or D654-A663, or a combination or an antigenic fragment of said region(s), to identify an antibody, binding agent or small molecule that specifically binds to the neomorphic region; b) performing a cell-based assay with the antibody, binding agent or small molecule identified in step a), to identify an antibody, binding agent or small molecule that modulates one or more non-canonical activities of a human GlyRS mutant associated with Charcot-Marie-Tooth Disease Type 2D or Distal Spinal Muscular Atrophy Type V (dSMA-V); c) optionally assessing the structure-activity relationship (SAR) of the antibody, binding agent or small molecule identified in step b), to correlate its structure with modulation of the non-canonical activity, and optionally derivatizing the antibody, binding agent or small molecule to alter its ability to modulate the non-canonical activity; and d) producing sufficient amounts of the antibody, binding agent or small molecule identified in step b), or the derivatized antibody, binding agent or small molecule step c), for use in humans, thereby manufacturing the pharmaceutical composition.

In certain embodiments, the polypeptide is a fusion protein that comprises at least one heterologous sequence and one or more of said neomorphic regions. In particular embodiments, said polypeptide consists essentially of A57-A663, A57-A83, L129-D161, N208-Y320, V366-H378, P518-M531, L584-Y604, F620-R635, or D654-A663 of human GlyRS, or combination or an antigenic fragment thereof. In certain embodiments, said binding agent is selected from the group consisting of adnectins, anticalins, avimers, DARPins, and aptamers. Certain binding agents include one or more soluble isoforms of a neuropilin transmembrane receptor, such as a soluble isoform of NRP1. In some embodiments, said antibody, binding agent or small molecule fully or partially antagonizes an interaction between a human GlyRS mutant associated with a Charcot-Marie-Tooth Disease Type 2D and its cellular binding partner(s).

Also included are methods of blocking or reducing neuropilin activity, for example, in a neuropilin-expressing cell, comprising contacting the neuropilin with a human glycyl-tRNA synthetase (GlyRS) mutant polypeptide, where the GlyRS mutant polypeptide exhibits specific binding to the neuropilin, thereby reducing neuropilin activity. In some aspects, affinity of the GlyRS mutant for neuropilin is greater than affinity of wild-type human GlyRS for neuropilin by at least about 1.5× to about 100× or more. In certain embodiments, the GlyRS mutant competitively inhibits binding of neuropilin to one or more neuropilin ligand(s). In certain embodiments, one or more neuropilin ligand(s) comprise a vascular endothelial growth factor (VEGF), a semaphorin, a placental growth factor, a heparin-binding protein, fibroblast growth factor-2, or hepatocyte growth factor. In specific embodiments, the VEGF is VEGF-165 or VEGF-B. In some embodiments, the semaphorin is semaphorin-3A (sema3A). In certain embodiments, neuropilin activity is associated with one or more of angiogenesis, cell migration, cell invasion, cell metastasis, or cell adhesion.

In certain embodiments, the GlyRS mutant is a disease-associated mutant. In particular embodiments, the GlyRS mutant is a CMT-associated mutant. In certain embodiments, the GlyRS mutant comprises one or more exposed neomorphic regions defined by amino acid residues A57-A83, L129-D161, N208-Y320, V366-H378, P518-M531, L584-Y604, F620-R635, and D654-A663 of full-length human GlyRS (SEQ ID NO:1). In certain embodiments, the GlyRS mutant is full-length. In some embodiments the GlyRS mutant comprises one or more of A57V, E71G, L129P, C157R, P234KY, G240R, P244L, I280F, H418R, D500N, G526R, S581L, or G598A relative to wild-type human GlyRS. In certain embodiments, the GlyRS mutant consists essentially of residues A57-A663 of wild-type GlyRS (SEQ ID NO:1), or an antigenic fragment thereof. In certain embodiments, the GlyRS mutant consists essentially of residues A57-A83, L129-D161, N208-Y320, V366-H378, P518-M531, L584-Y604, F620-R635, or D654-A663 of wild-type GlyRS, or an antigenic fragment thereof. In certain embodiments, the GlyRS mutant consists essentially of residues F79-A83, M227-L257, I232-N253, L258-E279, F147-K150, or E515-M531 of wild-type GlyRS. In specific embodiments, the GlyRS mutant consists of residues F79-A83, M227-L257, I232-N253, L258-E279, F147-K150, or E515-M531 of wild-type GlyRS. In particular embodiments, the GlyRS mutant consists of residues A57-A663, A57-A83, L129-D161, N208-Y320, V366-H378, P518-M531, L584-Y604, F620-R635, or D654-A663 of wild-type GlyRS. In certain embodiments, the GlyRS mutant comprises a heterologous fusion partner. Often, the GlyRS mutant is capable of specifically binding to neuropilin. In certain embodiments, the neuropilin is neuropilin-1 (NRP-1).

In certain embodiments, the neuropilin activity is in a subject, and the method comprises administering the GlyRS mutant polypeptide to the subject. Hence, also included are methods for treating the subject, wherein the subject has a disease or condition associated with increased neuropilin activity or expression, and/or increased activity or expression of a neuropilin ligand, optionally a VEGF and/or a semaphorin. In certain embodiments, the increased neuropilin activity is associated with one or more of angiogenesis, cell migration, cell invasion, cell metastasis, or cell adhesion. In particular embodiments, the subject has a cancer, optionally a metastatic cancer. In certain embodiments, the cancer is one or more of prostate cancer, breast cancer, colon cancer, rectal cancer, lung cancer, astrocytoma, ovarian cancer, testicular cancer, stomach cancer, bladder cancer, pancreatic cancer, liver cancer, kidney cancer, brain cancer, melanoma, non-melanoma skin cancer, bone cancer, lymphoma, leukemia, thyroid cancer, endometrial cancer, multiple myeloma, acute myeloid leukemia, neuroblastoma, glioblastoma, or non-Hodgkin's lymphoma.

Also included are methods of identifying an antibody, binding agent or small molecule that reduces binding between a glycyl-tRNA synthetase (GlyRS) mutant and a neuropilin polypeptide, wherein the GlyRS mutant comprises one or more exposed neomorphic regions, comprising a) combining a first polypeptide that comprises an exposed neomorphic region of human GlyRS, where said neomorphic region is one or more of A57-A663, A57-A83, L129-D161, N208-Y320, V366-H378, P518-M531, L584-Y604, F620-R635, or D654-A663, or a combination or an antigenic fragment of said region(s), with at least one test antibody, binding agent or small molecule, and a neuropilin polypeptide under suitable conditions, and b) detecting or determining ability of the test antibody, binding agent or small molecule to reduce binding between the first polypeptide and the neuropilin polypeptide, relative to a control sample without the test antibody, binding agent or small molecule, where reduced binding is statistically significant, thereby identifying an antibody, binding agent or small molecule that reduces binding between a GlyRS mutant and neuropilin transmembrane receptor.

In certain embodiments, said first polypeptide is a fusion protein that comprises at least one heterologous sequence and one or more of said neomorphic regions. In certain embodiments, said first polypeptide consists essentially of A57-A663, A57-A83, L129-D161, N208-Y320, V366-H378, P518-M531, L584-Y604, F620-R635, or D654-A663 of human GlyRS, or combination or an antigenic fragment thereof. In certain embodiments, said binding agent is selected from the group consisting of adnectins, anticalins, avimers, DARPins, and aptamers. In some embodiments, said antibody, binding agent or small molecule fully or partially antagonizes an interaction between a human disease-associated GlyRS mutant and the neuropilin. In certain embodiments, the antibody, binding agent or small molecule fully or partially antagonizes an interaction between a human GlyRS mutant associated with a Charcot-Marie-Tooth Disease Type 2D and the neuropilin.

In particular embodiments, said neuropilin polypeptide is expressed on the surface of a cell. In certain embodiments, the neuropilin is anchored to a solid substrate, and the first polypeptide and the test antibody, binding agent or small molecule are provided in solution. In some embodiments, the first polypeptide is bound to a solid substrate, and the neuropilin polypeptide and the test antibody, binding agent or small molecule are provided in solution. In certain embodiments, said neuropilin polypeptide is neuropilin-1 (NRP-1), or a polypeptide comprising an extracellular domain thereof.

Also included are antibodies or antigen-binding fragments thereof that exhibit binding specificity for one or more neuropilin(s), including, for example, a neuropilin transmembrane receptor, and which competitively inhibit binding between the neuropilin(s) and a disease-associated human glycyl-tRNA synthetase. Some embodiments include binding agents or small molecules that exhibit binding specificity for one or more neuropilin(s), including, for example, a neuropilin transmembrane receptor, and which competitively inhibit binding between the neuropilin and a disease-associated human glycyl-tRNA synthetase.

Also included are methods of reducing or ameliorating a symptom of Charcot-Marie-Tooth Disease Type 2D or Distal Spinal Muscular Atrophy Type V (dSMA-V) disease, comprising administering to a subject an antibody or antigen-binding fragment, a binding agent, or a small molecule that exhibits binding specificity for a neuropilin, and which competitively inhibits binding between the neuropilin and a disease-associated GlyRS mutant, where said subject has a GlyRS disease-associated mutation. In certain embodiments, said disease associated mutation is A57V, E71G, L129P, C157R, P234KY, G240R, P244L, I280F, D418R, D500N, G526R, S581L, or G598A.

### SEQUENCE LISTING

SEQ ID NO:1 is the full-length amino acid sequence of human glycyl-tRNA synthetase (GlyRS).

SEQ ID NO:2 is a polynucleotide sequence that encodes the human GlyRS of SEQ ID NO:1.

SEQ ID NO:3 is an exemplary amino acid sequence of a poly-histidine tag.

SEQ ID NO:4 is the amino acid sequence of human neuropilin-1 isoform a

SEQ ID NO:5 is the amino acid sequence of human neuropilin-1 isoform b.

SEQ ID NO:6 is the amino acid sequence of human neuropilin-1 isoform c.

SEQ ID NO:7 is the amino acid sequence of human neuropilin-1 soluble isoform 11.

SEQ ID NO:8 is the amino acid sequence of human neuropilin-1 soluble isoform 12.

SEQ ID NO:9 is the amino acid sequence of human neuropilin-1 isoform CRA_a.

SEQ ID NO:10 is the amino acid sequence of human neuropilin-2 isoform a.

SEQ ID NO:11 is the amino acid sequence of human neuropilin-2 isoform 3.

SEQ ID NO:12 is the amino acid sequence of human neuropilin-2 isoform 5.

SEQ ID NO:13 is the amino acid sequence of human neuropilin-2 isoform 1.

SEQ ID NO:14 is the amino acid sequence of human neuropilin-2 isoform 6.

SEQ ID NO:15 is the amino acid sequence of human neuropilin-2 isoform 4.

SEQ ID NO:16 is the amino acid sequence of human neuropilin-2 isoform 2.

SEQ ID NO:17 is the amino acid sequence of human neuropilin-2 soluble isoform 9.

### DETAILED DESCRIPTION OF THE INVENTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “antagonist” includes to a molecule that reduces or attenuates a CMT-associated non-canonical biological activity a GlyRS polypeptide, such as a GlyRS mutant associated with CMT. Antagonists may include proteins such as antibodies, nucleic acids, carbohydrates, small molecules, or any other compound or composition that modulates the activity of a GlyRS mutant or its binding partner, either by directly interacting with the GlyRS mutant or its binding partner or by acting on components of the biological pathway in which the GlyRS mutant participates. Included are partial and full antagonists.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “endotoxin free” or “substantially endotoxin free” relate generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art. Also included are methods of producing polypeptides such as antibodies in and isolating them from eukaryotic cells such as mammalian cells reduce if not eliminate the risk of endotoxins being present in a composition of the invention. Preferred are methods of producing polypeptides in and isolating them from serum free cells.

Endotoxins can be detected using routine techniques known in the art. For example, the Limulus Ameobocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/ml. Typically, 1 ng lipopolysaccharide (LPS) corresponds to about 1-10 EU.

In certain embodiments, the “purity” of any given agent (e.g., antibody, polypeptide binding agent) in a composition may be specifically defined. For instance, certain compositions may comprise an agent that is at least 80, 85, 90, 91, 92, 93, 94, 95, 96, 97, 98, 99, or 100% pure, including all decimals in between, as measured, for example and by no means limiting, by high pressure liquid chromatography (HPLC), a well-known form of column chromatography used frequently in biochemistry and analytical chemistry to separate, identify, and quantify compounds.

As used herein, the terms “function” and “functional” and the like refer to a biological, enzymatic, or therapeutic function.

By “isolated” is meant material that is substantially or essentially free from components that normally accompany it in its native state. For example, an “isolated polynucleotide,” as used herein, includes a polynucleotide that has been purified from the sequences that flank it in its naturally-occurring state, e.g., a DNA fragment which has been removed from the sequences that are normally adjacent to the fragment. Alternatively, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances.

The term “neomorphic region” relates to an exposed region or surface of human glycyl-tRNA synthetase (GlyRS) associated with one or more dominant, non-canonical activities of a neuronal disease-associated GlyRS mutant. These neomorphic regions or surfaces are mostly or entirely hidden (e.g., they have reduced solvent exposure) in a properly folded wild-type GlyRS sequence, but show significantly increased solvent exposure due to altered folding of a neuronal disease-associated GlyRS mutant, such as a CMT-associated GlyRS mutant. Certain neomorphic “opened up” regions partially overlap with the dimerization interface, and provide a new surface for potential pathological interactions specific to neuronal diseases such as CMT. Examples of disease-associated GlyRS mutants are described elsewhere herein and known in the art. Non-limiting examples of neomorphic regions include A57-A663, A57-A83, L129-D161, N208-Y320, V366-H378, P518-M531, L584-Y604, F620-R635, and D654-A663 of human GlyRS (SEQ ID NO:1), and fragments of said region(s), including antigenic fragments. Antigenic fragments of a neomorphic region can be at least about 6-12 to 20 or more residues in length, including all integers in between. Also included are combinations of these neomorphic regions, such as A57-D161, A57-Y320, A57-H378, A57-M531, A57-Y604, A57-R635, L129-Y320, L129-H378, L129-M531, L129-Y604, L129-R635, L129-A663, N208-H378, N208-M531, N208-Y604, N208-R635, N208-A663, V366-M531, V366-Y604, V366-R635, V366-A663, P518-Y604, P518-R635, P518-A663, L584-R635, L584-A663, and F620-A663, and others, including fragments thereof.

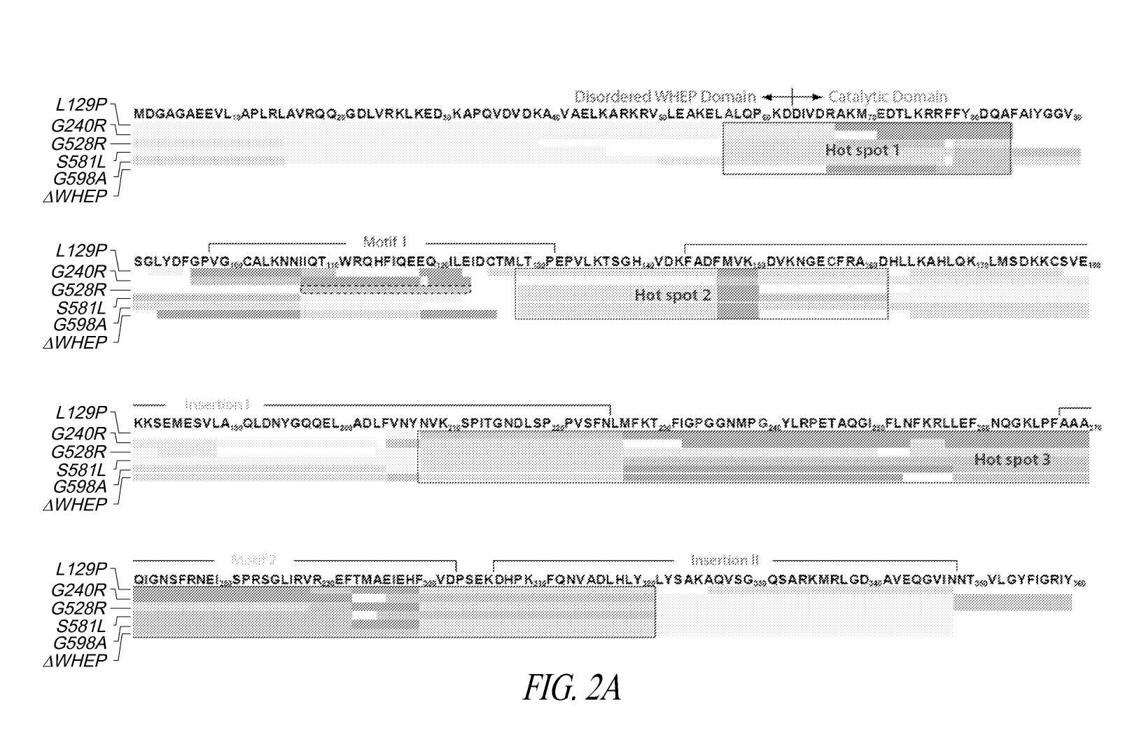

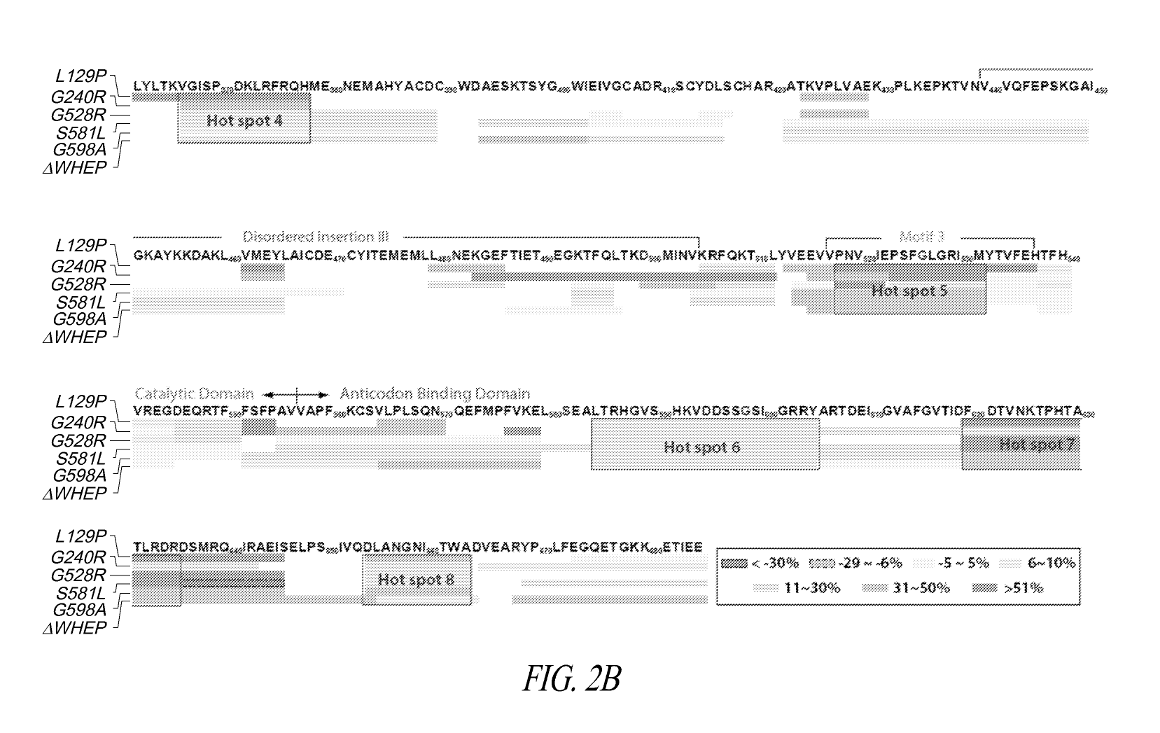

Examples of specific fragments of neomorphic regions include F79-A83, F78-T137, I108-E123, F224-L242, M227-L257, I232-N253, L252-E291, L258-R288, F147-K150, E515-M531, and R635-I645. Examples of specific neomorphic regions associated with GlyRS mutants include A57-A83, G97-T110, E119-S178, N208-Y320, A326-N348, L361-H378, K423-E429, V461-Y464, L480-F486, K505-P554, V564-N570, L584-Y604, F620-I645, and D654-A663 for the L129P of GlyRS; A57-A83, G97-E123, F147-L189, F204-Y320, N348-H378, V461-Y464, K483-M531, D545-R642, and D654-E685 for the G240R mutant of GlyRS; A57-A83, L129-K150, S183-V188, N208-Y320, N348-D389, K423-E429, L480-E485, D500-L511, P518-M531, T538-F550, L584-Y604, F620-I645, D654-A663 for the G526R mutant of GlyRS; A57-107, L129-D161, N208-Y320, V366-1402, K493-Q496, V513-M531, A555-R635, and D654-E685 for the S581L mutant of GlyRS; and A57-N106, L129-L203, N208-Y320, V366-D389, A421-Y464, E504-M531, F551-I645, and D654-A663 for the G598A mutant of GlyRS. Other neomorphic regions and their fragments are described, for example, in FIG. 2. For instance, regions characterized as “31-50%” or “>51%” can be included as neomorphic regions. Regions characterized as “29-6%” can also be characterized as neomorphic regions.

“Non-canonical” activity as used herein, refers generally to an activity possessed by a GlyRS polypeptide that is other than aminoacylation and, more specifically, other than the addition of its cognate amino acid onto its cognate tRNA molecule. Non-limiting examples of non-canonical activities include extracellular signaling, RNA-binding, modulation of cell proliferation, modulation of cell migration, modulation of cell differentiation, modulation of apoptosis or other forms of cell death, modulation of cell signaling, modulation of cell binding, modulation of cellular metabolism, modulation of cytokine production or activity, modulation of cytokine receptor activity, modulation of inflammation, and the like. Certain of these non-canonical activities may be related to the pathology of various diseases described herein and known in the art, such as CMT and Distal Spinal Muscular Atrophy Type V (dSMA-V), including, for example, activities related to neurite distribution defects and axonal degeneration, among others. Some non-canonical activities of disease-associated GlyRS relate to modulating (e.g., reducing, enhancing) the activity or activation of neuropilin transmembrane receptors, such as neuropilin-1, and/or modulating the activity of one or more of neuropilin ligands by altering (e.g., inhibiting) their interaction with neuropilin. Examples of such ligands include vascular endothelial growth factors (VEGFs), hepatocyte growth factor, placental growth factors (PGFs), semaphorins, among others described herein and known in the art. Specific neuropilin ligands include the VEGF-165 isoform, VEGF-B, the PLGF-2 isoform, and semaphorin-3A. One specific non-canonical activity of disease-associated GlyRS includes the inhibition of neuropilin-induced neurite outgrowth in cells.

The term “half maximal effective concentration” or “EC₅₀” refers to the concentration of an antibody or other agent described herein at which it induces a response halfway between the baseline and maximum after some specified exposure time; the EC₅₀ of a graded dose response curve therefore represents the concentration of a compound at which 50% of its maximal effect is observed. In certain embodiments, the EC₅₀ of an agent provided herein is indicated in relation to a “non-canonical” activity, as noted above, for example, a non-canonical activity related to symptoms or pathology of CMT. EC₅₀ also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC₅₀” and the Hill slope, or it can be determined from the data directly, using routine knowledge in the art. In some embodiments, the EC₅₀ of an antibody or other agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, or 100 nM. Preferably, biotherapeutic compositions will have an EC₅₀ value of about 1 nM or less.

The term “modulating” includes “increasing” or “stimulating,” as well as “decreasing” or “reducing,” typically in a statistically significant or a physiologically significant amount as compared to a control. An “increased” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the amount produced by no composition (the absence of an agent or compound) or a control composition. A “decreased” or reduced amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition (the absence of an agent or compound) or a control composition, including all integers in between. As one non-limiting example, a control in comparing canonical and non-canonical activities could include the activity (e.g., antagonist activity) or binding specificity of an antibody or binding agent towards a disease-associated GlyRS mutant of interest relative to a wild-type human GlyRS. Other examples of “statistically significant” amounts are described herein.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated, for example, by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The comparison of sequences and determination of percent identity between two sequences can be accomplished using a mathematical algorithm. In a preferred embodiment, the percent identity between two amino acid sequences is determined using the Needleman and Wunsch, (1970, J. Mol. Biol. 48: 444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4: 11-17), which has been incorporated into the ALIGN program (version 2.0), using a PAM120 weight residue table, a gap length penalty of 12 and a gap penalty of 4.

The term “solubility” refers to the property of an antibody, peptide, or other agent provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH. In certain embodiments, solubility is measured in water or a physiological buffer such as PBS. In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, or 30 mg/ml at room temperature or at 37° C.

A “subject,” as used herein, includes any animal that exhibits a symptom, or is at risk for exhibiting a symptom, of one or more diseases such as distal spinal muscular atrophies (dSMA) and distal hereditary motor neuropathies (dHMN), which are preferably associated with one or more GlyRS mutations. Examples of neuronal disease-associated GlyRS mutants include, without limitation, A57V, E71G, L129P, C157R, P234KY, G240R, P244L, I280F, H418R, D500N, G526R, S581L, and G598A mutants of wild-type GlyRS. Specific examples of diseases, including mutant GlyRS-associated diseases, include CMT Type 1, CMT Type 2, CMT Type 2D, and dSMA Type V, among others described herein and known in the art. Also included are subjects for which it is desirable to profile presence and/or levels of disease-associated GlyRS mutants, for diagnostic or other purposes. In certain aspects, a subject includes any animal having a disease or condition associated with increased or aberrant activity of a neuropilin-related pathway, as described herein and known in the art. Suitable subjects (patients) include laboratory animals (such as mouse, rat, rabbit, or guinea pig), farm animals, and domestic animals or pets (such as a cat or dog). Non-human primates and, preferably, human patients, are included.

“Treatment” or “treating,” as used herein, includes any desirable effect on the symptoms or pathology of a disease or condition, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (3^{rd} Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R.I. (2005) Culture of Animal Cells, a Manual of Basic Technique, 5^{th} Ed. Hoboken N.J., John Wiley & Sons; B. Perbal, A Practical Guide to Molecular Cloning (3^{rd} Edition 2010); Farrell, R., RNA Methodologies: A Laboratory Guide for Isolation and Characterization (3^{rd} Edition 2005).

All publications, patents and patent applications cited herein are hereby incorporated by reference in their entirety.

### Polypeptides of Human GlyRS

As noted above, the present invention is based in part on the discovery of neomorphic regions human glycyl-tRNA synthetase (GlyRS), one or more of which are typically exposed on the surface of neuronal-disease related mutants of GlyRS but not wild-type GlyRS, and thus associate with the neuronal pathology of disease-associated mutant GlyRS polypeptides. Accordingly such disease-associated mutant GlyRS polypeptides comprise at least one disease associated mutation. In some embodiments, such disease-associated mutant GlyRS polypeptides differ from SEQ. ID. No. 1 by at least one amino acid selected from the group consisting of A57V, E71G, L129P, C157R, P234KY, G240R, P244L, I280F, H418R, D500N, G526R, S581L, and G598A. In certain embodiments, the disease associated mutation is selected from the group consisting of L129P, G240R, G526R, S581L, and G598A.

Embodiments of the present invention therefore include, for example, polypeptides and peptides that consist or consist essentially of the human glycyl-tRNA synthetase neomorphic regions described herein, and fusion polypeptides that comprise such regions. The GlyRS polypeptides, peptides, and fusion proteins of the invention can be used, for example, in any of the diagnostic and/or drug discovery methods described herein.

GlyRS mutant polypeptides, peptides, and fusion proteins can also be used in certain therapeutic methods described herein, including methods of treating diseases or conditions associated with aberrant activity or activation of a neuropilin transmembrane receptor pathway, (i.e., “neuropilin-related diseases”) such as cancers and other conditions described herein. In certain aspects, these and related GlyRS polypeptides or peptides may have an affinity for neuropilin transmembrane receptors that is greater than the affinity of wild-type human GlyRS for a comparable neuropilin transmembrane receptor by at least about 1.5× to about 100× or about 1000× or more, including all ranges and integers in between. Likewise, certain GlyRS mutants provided herein can be characterized by their ability to competitively inhibit binding between a neuropilin transmembrane receptor such as NRP-1 and one or more of its ligands, including VEGFs, PGFs, HU, FGF-2, and semaphorins. Specific examples of neuropilin ligands include the VEGF-165 isoform, VEGF-B, the PLGF-2 isoform, and semaphorin-3A (sema3A), among others described herein and known in the art.

Certain embodiments relate to isolated polypeptides or peptides, consisting or consisting essentially of residues A57-A663 of wild-type GlyRS (SEQ ID NO:1), or a neomorphic or antigenic fragment thereof. In some embodiments, the isolated polypeptides or peptides comprise at least one disease associated mutation. Accordingly in some aspects, such isolated polypeptides or peptides differ from SEQ ID NO:1 by at least one amino acid selected from the group consisting of A57V, E71G, L129P, C157R, P234KY, G240R, P244L, I280F, H418R, D500N, G526R, S581L, and G598A. Specific examples of polypeptides consist or consist essentially of the neomorphic “hot spot” regions defined by residues A57-A83, L129-D161, N208-Y320, V366-H378, P518-M531, L584-Y604, F620-R635, and D654-A663 of wild-type GlyRS, or an antigenic fragment or combination of any of these regions. Examples of combinations of these regions include polypeptides or peptides that consist or consist essentially of residues A57-D161, A57-Y320, A57-H378, A57-M531, A57-Y604, A57-R635, L129-Y320, L129-H378, L129-M531, L129-Y604, L129-R635, L129-A663, N208-H378, N208-M531, N208-Y604, N208-R635, N208-A663, V366-M531, V366-Y604, V366-R635, V366-A663, P518-Y604, P518-R635, P518-A663, L584-R635, L584-A663, and F620-A663, and others, including neomorphic fragments thereof.

Certain specific peptides consist or consisting essentially of residues F79-A83, F78-T137, I108-E123, F224-L242, M227-L257, I232-N253, L252-E291, L258-R288, F147-K150, E515-M531, or R635-I645 of wild-type GlyRS. Additional specific peptides consist or consist essentially of residues A57-A83, G97-T110, E119-S178, N208-Y320, A326-N348, L361-H378, K423-E429, V461-Y464, L480-F486, K505-P554, V564-N570, L584-Y604, F620-I645, D654-A663, G97-E123, F147-L189, F204-Y320, N348-H378, V461-Y464, K483-M531, D545-R642, D654-E685, L129-K150, S183-V188, N208-Y320, N348-D389, K423-E429, L480-E485, D500-L511, P518-M531, T538-F550, L584-Y604, F620-I645, D654-A663, A57-107, L129-D161, N208-Y320, V366-I402, K493-Q496, V513-M531, A555-R635, D654-E685, A57-N106, L129-L203, N208-Y320, V366-D389, A421-Y464, E504-M531, F551-I645, or D654-A663 of wild-type GlyRS. Typically, the polypeptide or peptide has at least one non-canonical activity relative to wild-type GlyRS, including, for example, a disease-associated activity, such as a CMT disease-associated activity.

The terms “polypeptide” and “protein” are used interchangeably herein to refer to a polymer of amino acid residues and to variants and synthetic analogues of the same. In certain embodiments, the term “peptide” refers to relatively short polypeptides, including peptides that consist of about 2, 3, 4, 5, 6, 7, 8, 9, 10, 11, 12, 13, 14, 15, 16, 17, 18, 19, 20, 25, 30, 35, 40, 45, or 50 amino acids, including all integers and ranges (e.g., 5-10, 8-12, 10-15) in between. Thus, these terms apply to amino acid polymers in which one or more amino acid residues are synthetic non-naturally occurring amino acids, such as a chemical analogue of a corresponding naturally occurring amino acid, as well as to naturally-occurring amino acid:polymers.

In general, polypeptides and fusion polypeptides (as well as their encoding polynucleotides) are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

As used herein, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivitization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

In certain aspects, the use of non-natural amino acids can be utilized to modify (e.g., increase) a selected non-canonical activity of a GlyRS polypeptide, or to alter the in vivo or in vitro half-life of the protein. Non-natural amino acids can also be used to facilitate (selective) chemical modifications (e.g., pegylation) of a GlyRS polypeptide or fusion protein, as described below. For instance, certain non-natural amino acids allow selective attachment of polymers such as PEG to a given protein, and thereby improve their pharmacokinetic properties.

Specific examples of amino acid analogs and mimetics can be found described in, for example, Roberts and Vellaccio, The Peptides: Analysis, Synthesis, Biology, Eds. Gross and Meinhofer, Vol. 5, p. 341, Academic Press, Inc., New York, N.Y. (1983), the entire volume of which is incorporated herein by reference. Other examples include peralkylated amino acids, particularly permethylated amino acids. See, for example, Combinatorial Chemistry, Eds. Wilson and Czarnik, Ch. 11, p. 235, John Wiley & Sons Inc., New York, N.Y. (1997), the entire book of which is incorporated herein by reference. Yet other examples include amino acids whose amide portion (and, therefore, the amide backbone of the resulting peptide) has been replaced, for example, by a sugar ring, steroid, benzodiazepine or carbo cycle. See, for instance, Burger's Medicinal Chemistry and Drug Discovery, Ed. Manfred E. Wolff, Ch. 15, pp. 619-620, John Wiley & Sons Inc., New York, N.Y. (1995), the entire book of which is incorporated herein by reference. Methods for synthesizing peptides, polypeptides, peptidomimetics and proteins are well known in the art (see, for example, U.S. Pat. No. 5,420,109; M. Bodanzsky, Principles of Peptide Synthesis (1st ed. & 2d rev. ed.), Springer-Verlag, New York, N.Y. (1984 & 1993), see Chapter 7; Stewart and Young, Solid Phase Peptide Synthesis, (2d ed.), Pierce Chemical Co., Rockford, Ill. (1984), each of which is incorporated herein by reference). Accordingly, the polypeptides of the present invention may be composed of naturally occurring and non-naturally occurring amino acids as well as amino acid analogs and mimetics.

Also included are “variants” of these mutant GlyRS polypeptides and peptides described herein. The term “variant” refers to polypeptides that are distinguished from a reference polypeptide, such as SEQ ID No:1, or any of the mutant GlyRS polypeptides disclosed herein by the addition, deletion, and/or substitution of at least one amino acid residue, and which typically retain (e.g., mimic) or modulate (e.g., antagonize) one or more non-canonical activities of a reference polypeptide.

In certain embodiments, a polypeptide variant is distinguished from a reference polypeptide by one or more substitutions, which may be conservative or non-conservative, as described herein and known in the art. In certain embodiments, the polypeptide variant comprises conservative substitutions and, in this regard, it is well understood in the art that some amino acids may be changed to others with broadly similar properties without changing the nature of the activity of the polypeptide.

In certain embodiments, a variant polypeptide includes an amino acid sequence having at least about 50%, 55%, 60%, 65%, 70%, 75%, 80%, 85%, 90%, 91%, 92%, 93%, 94%, 95%, 96%, 97%, 98% or more sequence identity or similarity to a corresponding sequence of a neomorphic GlyRS reference polypeptide or peptide, (for example, compared to a polypeptide or peptide which differs from SEQ ID NO:1 by at least one amino acid selected from the group consisting of A57V, E71G, L129P, C157R, P234KY, G240R, P244L, I280F, H418R, D500N, G526R, S581L, and G598A) as described herein, and substantially retains the non-canonical activity of that reference polypeptide. Also included are sequences differing from the reference sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids (e.g., heterologous or non-GlyRS amino acids) but which retain the properties of the reference polypeptide. In certain embodiments, the amino acid additions or deletions occur at the C-terminal end and/or the N-terminal end of the reference polypeptide. In certain embodiments, the amino acid additions include 1, 2, 3, 4, 5, 6, 7, 8, 9, 10, 11, 12, 13, 14, 15, 16, 17, 18, 19, 20, 30, 40, 50 or more homologous (e.g., from the corresponding full-length GlyRS polypeptide) or heterologous (e.g., non-GlyRS sequences) residues that are proximal to the C-terminal end and/or the N-terminal end of the neomorphic GlyRS reference polypeptide.

In certain embodiments, variant polypeptides differ from the corresponding neomorphic GlyRS reference sequences by at least 1% but less than 20%, 15%, 10% or 5% of the residues. (If this comparison requires alignment, the sequences should be aligned for maximum similarity. “Looped” out sequences from deletions or insertions, or mismatches, are considered differences.) The differences are, suitably, differences or changes at a non-essential residue or a conservative substitution. In certain embodiments, the molecular weight of a variant GlyRS polypeptide differs from that of the reference polypeptide by about 1%, 2%, 3%, 4%, 5%, 6%, 7%, 8%, 9%, 10%, 11%, 12%, 13%, 14%, 15%, 16%, 17%, 18%, 19%, 20%, or more.

Also included are biologically active “fragments” of the neomorphic or mutant GlyRS polypeptides. Exemplary fragments include for example mutant GlyRS polypeptides lacking the WHEP domain, (comprising about amino acids 55 to 115 of SEQ ID NO: 1). In one aspect such WHEP domain deleted fragments also differ from SEQ. ID. No. 1 by at least one amino acid selected from the group consisting of L129P, C157R, P234KY, G240R, P244L, I280F, H418R, D500N, G526R, S581L, and G598A. Representative biologically active fragments generally participate in an interaction, e.g., an intramolecular or an inter-molecular interaction. An inter-molecular interaction can be a specific binding interaction or an enzymatic interaction. An inter-molecular interaction can be between a neomorphic GlyRS polypeptide and a cellular binding partner, such as a cellular receptor or other host molecule that participates in the non-canonical activity of the GlyRS polypeptide. Certain cellular binding partners include neuropilin transmembrane receptors, such as neuropilin-1 (NRP-1) and neuropilin-2 (NRP-2).

A biologically active fragment of a neomorphic or mutant GlyRS polypeptide can be a polypeptide fragment which is, for example, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 450, 500 or more contiguous or non-contiguous amino acids, including all integers (e.g., 101, 102, 103) and ranges (e.g., 50-100, 50-150, 50-200) in between, of the GlyRS neomorphic regions described herein. In certain embodiments, a biologically active fragment comprises a non-canonical activity-related sequence, domain, or motif. In certain embodiments, the C-terminal or N-terminal region of any GlyRS neomorphic region may be truncated by about 1, 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, 250, 300, 350, 400, 450, 500 or more amino acids, or by about 10-50, 20-50, 50-100, 100-150, 150-200, 200-250, 250-300, 300-350, 350-400, 400-450, 450-500 or more amino acids, including all integers and ranges in between (e.g., 101, 102, 103, 104, 105), so long as the truncated GlyRS polypeptide retains the non-canonical activity of the reference polypeptide. Typically, the biologically-active fragment has no less than about 1%, 10%, 25%, or 50% of an activity of the biologically-active (i.e., non-canonical activity) neomorphic GlyRS polypeptide from which it is derived.

In some embodiments, neomorphic or mutant GlyRS polypeptides, peptides, variants, and biologically active fragments thereof, bind to one or more cellular binding partners with an affinity of at least about or less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, or 200 nM. In some embodiments, the binding affinity of a GlyRS polypeptide for a selected cellular binding partner, particularly a binding partner that participates in a non-canonical activity, can be stronger than that of wild-type GlyRS, by at least about 1.5×, 2×, 2.5×, 3×, 3.5×, 4×, 4.5×, 5×, 6×, 7×, 8×, 9×, 10×, 15×, 20×, 25×, 30×, 40×, 50×, 60×, 70×, 80×, 90×, 100×, 200×, 300×, 400×, 500×, 600×, 700×, 800×, 900×, 1000× or more (including all integers in between). In some aspects, the mutant GlyRS will exhibit an apparent affinity for neuropilin-1 in a BIACORE assay of at least about 10 nM, at least about 20 nM, at least about 50 nM, at least about 100 nM, at least about 200 nM, or at least about 500 nM. In specific embodiments, a mutant GlyRS polypeptide comprises a mutation at residue L129 (optionally L129P) and specifically binds to neuropilin-1 with an affinity of about 30-50 nM, or about 30, 32, 36, 38, 40, 42, 44, 46, 48, 50 nM. In other embodiments, a mutant GlyRS polypeptide comprises a mutation at P234 (optionally P234KY) and specifically binds to neuropilin-1 with an affinity of about 150-170 nM, or about 150, 152, 154, 156, 158, 160, 162, 164, 166, 168, or 170 nM.

As noted above, a neomorphic or mutant GlyRS polypeptide may be altered in various ways including amino acid substitutions, deletions, truncations, and insertions. Methods for such manipulations are generally known in the art. For example, amino acid sequence variants of a given polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

Biologically active truncated and/or variant mutant GlyRS polypeptides may contain conservative amino acid substitutions at various locations along their sequence, as compared to a reference GlyRS amino acid residue. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices are known in the art (see e.g., PAM120 matrix and PAM250 matrix as disclosed for example by Dayhoff et al., 1978, A model of evolutionary change in proteins). Matrices for determining distance relationships In M. G. Dayhoff, (ed.), Atlas of protein sequence and structure, Vol. 5, pp. 345-358, National Biomedical Research Foundation, Washington D.C.; and by Gonnet et al., (Science, 256: 14430-1445, 1992), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behavior.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table A.

TABLE A
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, |
| | Lysine, Histidine |
| Small | Glycine, Serine, Alanine, Threonine, Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine |
| Residues that influence | Glycine and Proline |
| chain orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its non-canonical activity, as described herein. Conservative substitutions are shown in Table B under the heading of exemplary substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, (c) the bulk of the side chain, or (d) the biological function. After the substitutions are introduced, the variants are screened for biological activity.

TABLE B
Exemplary Amino Acid Substitutions

| Original | Exemplary | Preferred |
|---|---|---|
| Residue | Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm.C. Brown Publishers (1993).

Thus, a predicted non-essential amino acid residue in a given polypeptide is typically replaced with another amino acid residue from the same side chain family. Alternatively, mutations can be introduced randomly along all or part of a given coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined. A “non-essential” amino acid residue is a residue that can be altered from the reference sequence of an embodiment polypeptide without abolishing or substantially altering one or more of its activities. Suitably, the alteration does not substantially abolish one of these activities, for example, the activity is at least 20%, 40%, 60%, 70% or 80% 100%, 500%, 1000% or more of the reference GlyRS sequence. An “essential” amino acid residue is a residue that, when altered from the reference sequence of a neomorphic GlyRS polypeptide, results in abolition of an activity of the parent molecule such that less than 20% of the reference activity is present. For example, such essential amino acid residues include those that are conserved in GlyRS polypeptides across different species, including those sequences that are conserved in the active binding site(s) or motif(s) of GlyRS polypeptides from various sources.

The present invention also contemplates the use of neomorphic or mutant GlyRS chimeric or fusion proteins. As used herein, a “chimeric protein” or “fusion protein” includes a neomorphic or mutant GlyRS polypeptide or peptide linked to either another (same or different) neomorphic GlyRS polypeptide (e.g., to create multiple fragments), to a heterologous non-GlyRS polypeptide, or to both. A “heterologous polypeptide” refers to a polypeptide having an amino acid sequence corresponding to a protein which is different from a human GlyRS sequence, and which can be derived from the same or a different organism. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

The fusion partner may be designed and included for essentially any desired purpose provided they do not adversely affect the activity of the neomorphic or mutant GlyRS polypeptide. For example, in one embodiment, a fusion partner may comprise a sequence that assists in expressing the protein (an expression enhancer) at higher yields than the native recombinant protein. Other fusion partners may be selected so as to increase the solubility of the protein or to enable the protein to be targeted to desired intracellular compartments

The fusion protein can include a moiety which has a high affinity for a ligand. For example, the fusion protein can be a GST-GlyRS fusion protein in which the GlyRS sequences are fused to the C-terminus of the GST sequences. As another example, a GlyRS peptide or polypeptide may be fused to an affinity/and or epitope tag at the C-terminus, such as a poly-histidine tag such as a sequence comprising the sequence L-E-H-H-H-H-H-H (SEQ ID NO:3). Such fusion proteins can facilitate the purification and/or identification of a neomorphic GlyRS polypeptide. Selected moieties can also be used to attach the fusion protein to a surface, for example, to facilitate screening for agents that specifically bind to the neomorphic GlyRS polypeptide or peptide. Alternatively, the fusion protein can be an GlyRS protein containing a heterologous signal sequence at its N-terminus. In certain host cells, expression and/or secretion of GlyRS proteins can be increased through use of a heterologous signal sequence.

More generally, fusion to heterologous sequences, such as an Fc fragment, may be utilized to remove unwanted characteristics or to improve the desired characteristics (e.g., pharmacokinetic properties) of a neomorphic GlyRS polypeptide. For example, fusion to a heterologous sequence may increase chemical stability, decrease immunogenicity, improve in vivo targeting, and/or increase half-life in circulation of a GlyRS polypeptide.

Fusion proteins may generally be prepared using standard techniques. For example, DNA sequences encoding the polypeptide components of a desired fusion may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures, if desired. Such a peptide linker sequence is incorporated into the fusion protein using standard techniques well known in the art. Certain peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences may be operably linked to suitable transcriptional or translational regulatory elements. The regulatory elements responsible for expression of DNA are typically located 5′ to the DNA sequence encoding the first polypeptide. Similarly, stop codons required to end translation and transcription termination signals are present 3′ to the DNA sequence encoding the second polypeptide.

Certain embodiments of the present invention also contemplate the use of modified mutant GlyRS polypeptides and peptides, including modifications that improved the desired characteristics of the polypeptide or peptide, as described herein. Modifications include chemical and/or enzymatic derivatizations at one or more constituent amino acid, including side chain modifications, backbone modifications, and N- and C-terminal modifications including acetylation, hydroxylation, methylation, amidation, and the attachment of carbohydrate or lipid moieties, cofactors, and the like. Exemplary modifications also include pegylation (see, e.g., Veronese and Harris, Advanced Drug Delivery Reviews 54: 453-456, 2002, herein incorporated by reference).

In certain aspects, chemoselective ligation technology may be utilized to modify polypeptides of the invention, such as by attaching polymers in a site-specific and controlled manner. Such technology typically relies on the incorporation of chemoselective anchors into the protein backbone by either chemical or recombinant means, and subsequent modification with a polymer carrying a complementary linker. As a result, the assembly process and the covalent structure of the resulting protein—polymer conjugate may be controlled, enabling the rational optimization of drug properties, such as efficacy and pharmacokinetic properties (see, e.g., Kochendoerfer, Current Opinion in Chemical Biology 9:555-560, 2005).

The polypeptides and peptides described herein may be prepared by any suitable procedure known to those of skill in the art, such as by recombinant techniques. For example, polypeptides may be prepared by a procedure including the steps of: (a) preparing a construct comprising a polynucleotide sequence that encodes a desired polypeptide and that is operably linked to a regulatory element; (b) introducing the construct into a host cell; (c) culturing the host cell to express the polypeptide; and (d) isolating the polypeptide from the host cell. In illustrative examples, the nucleotide sequence encodes at least a biologically active portion of a neomorphic region described herein, or a biologically active variant or fragment thereof. Recombinant polypeptides can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6.

In addition to recombinant production methods, polypeptides of the invention, and fragments thereof, may be produced by direct peptide synthesis using solid-phase techniques (Merrifield, J. Am. Chem. Soc. 85:2149-2154 (1963)). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the desired molecule.

### Antibodies

According to one aspect, the present invention provides antibodies and antigen-binding fragments thereof that exhibit binding specificity for at least one neomorphic region of human glycyl-tRNA synthetase (GlyRS), or to an antigenic fragment, variant or derivative thereof, and compositions and methods of using the same. The term “antibody” relates to an immunoglobulin whether natural or partly or wholly synthetically produced. The term also covers any polypeptide or protein having a binding domain which is, or is homologous to, an antigen-binding domain. CDR grafted antibodies are also contemplated by this term.

The term “antigen-binding portion of an antibody,” “antigen-binding fragment,” “antigen-binding domain,” “antibody fragment” or a “functional fragment of an antibody” are used interchangeably in the present invention to include one or more fragments of an antibody that retain the ability to specifically bind to an antigen (see, e.g., Holliger et al., Nature Biotech. 23 (9): 1126-1129 (2005)). Partly because certain of the antibodies described herein are relatively specific for disease-associated GlyRS mutants, including mutants associated with various diseases such as Charcot-Marie-Tooth (CMT) diseases, these antibodies can be used in a variety of disease related therapeutic, diagnostic, drug discovery, or protein expression/purification methods and compositions provided herein.

As noted above, antibodies (or antigen binding fragments) of the present invention typically exhibit binding specificity for one or more GlyRS neomorphic regions described herein. These neomorphic regions typically show greater solvent exposure on the surface of disease-associated GlyRS mutants than on the surface of wild-type GlyRS. The antibodies described herein thus exhibit binding specificity for one or more disease-associated GlyRS mutants.

In some embodiments, an antibody may exhibit binding specificity for both wild type human GlyRS and a disease-associated mutant human GlyRS; however, because the neomorphic regions described herein are typically not significantly solvent exposed in a properly folded wild-type GlyRS protein, the affinity of the antibody for a given mutant human GlyRS is typically different than, for example, stronger or greater than, than its affinity for wild type human GlyRS, for instance, by at least about 2× to at least about 100× or to at least about 1000× or more.

Certain antibodies of the present invention can thus distinguish between a disease-associated GlyRS mutant, such as CMT-associated GlyRS mutant, and a corresponding wild-type GlyRS, typically by binding with greater affinity to the mutant GlyRS (e.g., via one or more of its exposed neomorphic regions) than to the corresponding full-length GlyRS. In some embodiments the antibody is specific for the disease associated GlyRS mutant, and does not bind substantially to the wild type GlyRS. For example, as noted above, antibodies may exhibit binding specificity for one or more non-solvent exposed faces that are exposed in the GlyRS mutant but not in the full-length GlyRS. An antibody thus may exhibit binding specificity for a properly folded, disease-associated GlyRS mutant, preferably where the folding of that mutant is characterized, for example, by its native or most stable three-dimensional conformation in a cell or a physiological solution. In some embodiments the antibody binds to a subset of the disease associated mutants, but not wild type GlyRS.

It should also be noted that an antibody may exhibit binding specificity for a denatured or partially denatured version of wild-type GlyRS, in part because the de-natured protein may allow access to the otherwise (relatively) hidden neomorphic regions. In specific embodiments, however, an antibody does not show significant specific binding to a correctly folded version of wild-type GlyRS, where folding of that wild-type GlyRS is characterized, for example, by its native or most stable three-dimensional conformation in a cell or a physiological solution. Antibodies may thus bind to unique three-dimensional structures that result from differences in folding between a given disease-associated GlyRS mutant and wild-type GlyRS. Such differences in folding may be localized (e.g., to a specific domain or region) or globalized. As one example, the folding of a mutant GlyRS may generate unique solvent-exposed, continuous or discontinuous epitopes that are not solvent-exposed in the wild-type human GlyRS.

Antibodies of the present invention may also exhibit binding specificity for peptides that consist or consist essentially of a GlyRS neomorphic region described herein, and/or heterologous fusion proteins comprising the same, e.g., one or more GlyRS neomorphic regions fused to one or more non-GlyRS polypeptide sequences. Examples of neomorphic regions or exposed faces include the region within A57-A663 and the “hot spot” regions defined by A57-A83, L129-D161, N208-Y320, V366-H378, P518-M531, L584-Y604, F620-R635, or D654-A663 of full-length GlyRS, and antigenic fragments of said regions. Also included are any combinations of these regions, e.g., A57-D161, A57-Y320, A57-H378, A57-M531, A57-Y604, A57-R635. These neomorphic “hot spot” regions are shared by a variety of disease-associated GlyRS mutants, including numerous CMT-associated GlyRS mutants. Additional examples of neomorphic regions include fragments of these “hot spots,” such as F79-A83, M227-L257, I232-N253, L258-R288, F147-K150, and E515-M531. Other neomorphic regions and their fragments are described, for example, in FIG. 2. For instance, regions characterized as “31-50%” or “>51%” can be included as neomorphic regions.

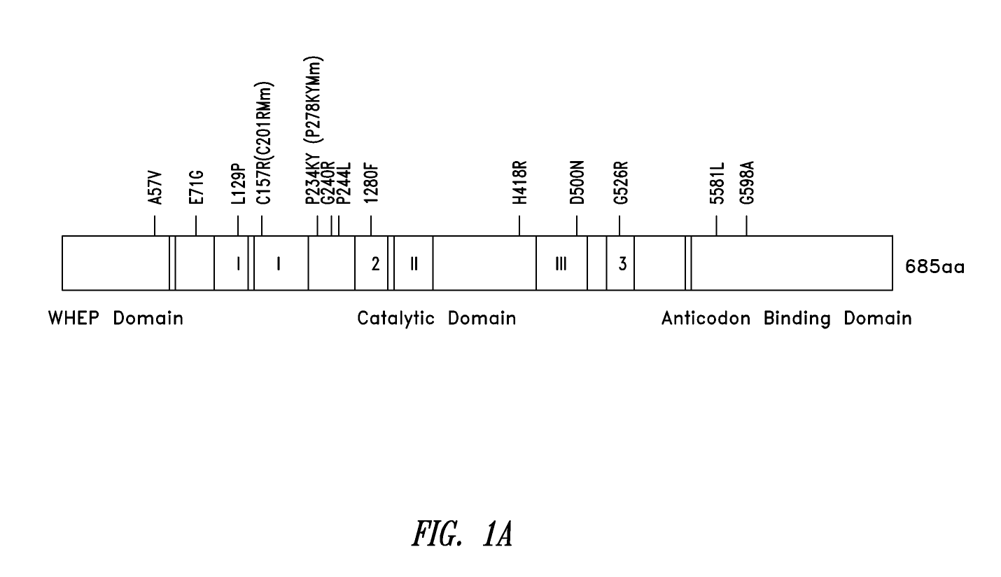

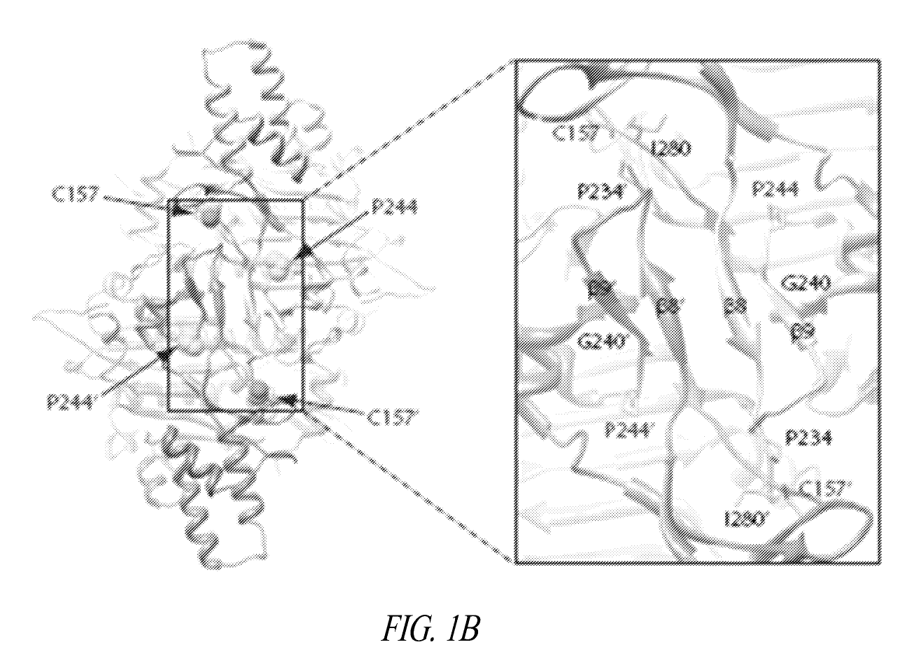

In certain embodiments, antibodies of the present invention may exhibit binding specificity (cross-reactivity) for one or more specific disease-associated GlyRS mutants, but not a different disease-associated GlyRS mutant (or with lesser affinity to that different GlyRS mutant). Such comparisons are preferably made when the proteins are properly folded. Here, even though disease-associated GlyRS mutants share a number of hot spot neomorphic regions, some neomorphic regions are relatively unique to, or have increased surface exposure in, a given disease-associated GlyRS mutants relative to others (see FIG. 2). Hence, certain antibodies may be used to treat and/or diagnose specific types of diseases, corresponding to specific GlyRS mutants. For example, the L129P mutant is associated with increased solvent exposure of amino acid residues A57-A83, G97-T110, E119-S178, N208-Y320, A326-N348, L361-H378, K423-E429, V461-Y464, L480-F486, K505-P554, V564-N570, L584-Y604, F620-I645, and D654-A663. As another example, the G240R mutant is associated with increased solvent exposure of residues A57-A83, G97-E123, F147-L189, F204-Y320, N348-H378, V461-Y464, K483-M531, D545-R642, and D654-E685. The G526R mutant is associated with increased solvent exposure of residues A57-A83, L129-K150, S183-V188, N208-Y320, N348-D389, K423-E429, L480-E485, D500-L511, P518-M531, T538-F550, L584-Y604, F620-I645, and D654-A663. The S581L mutant is associated with increased solvent exposure of residues A57-107, L129-D161, N208-Y320, V366-I402, K493-Q496, V513-M531, A555-R635, and D654-E685. The G598A mutant is associated with increased solvent exposure of residues A57-N106, L129-L203, N208-Y320, V366-D389, A421-Y464, E504-M531, F551-I645, and D654-A663. Examples of disease-associated GlyRS mutants include A57V, E71G, L129P, C157R, P234KY, G240R, P244L, I280F, H418R, D500N, G526R, S581L, and G598A mutants. See, e.g., FIG. 1A; and Nangle et al., PNAS USA. 104:11239-11244, 2007).

Antibodies may also exhibit binding specificity for GlyRS mutants having a full or partial deletion of the WHEP domain. Without wishing to be bound by any one theory, the accompanying Examples suggest that deletion of the WHEP domain may induce a conformational change resembling that of the CMT-associated GlyRS mutations, exposing one or more neomorphic regions described herein. Similar to above, these and related antibodies may be able to distinguish between a WHEP domain mutant of GlyRS and wild-type GlyRS, for example, by having significantly greater affinity for at least one exposed neomorphic region of a WHEP domain (deletion) mutant than for wild-type GlyRS; preferably, affinity comparisons are made between properly folded GlyRS proteins.

Because certain disease-associated GlyRS mutants lead to alteration of the dimer interface, and increase in monomer forms of GlyRS, certain antibodies may exhibit binding specificity for the monomer but not the dimer form of human GlyRS. For certain antibodies, their affinity for the monomer form of GlyRS is stronger than their affinity for the dimer form of GlyRS by at least about 2× to at least about 100× or to at least about 1000× or more.

In some embodiments, antibodies provided herein do not form aggregates, have a desired solubility, and/or have an immunogenicity profile that is suitable for use in humans, as described herein and known in the art. Also included are antibodies that are suitable for production work, such as to purify a GlyRS mutant described herein. Preferably, active antibodies can be concentrated to at least about 10 mg/ml and optional formulated for biotherapeutic or diagnostic uses.

In certain embodiments, antibodies are effective for modulating one or more of the non-canonical activities mediated by a mutant GlyRS that is associated with a disease, such as neuronal disease. A specific example of a non-canonical activity is the modulation (inhibition or activation) of one or more neuropilin transmembrane receptors, such as NRP-1 and NRP-2. Certain antibodies may reduce the GlyRS-mediated modulation (e.g., inhibition) of neuropilins. In certain embodiments, for example, the antibody is one that binds to mutant GlyRS polypeptide and/or its cellular binding partner, inhibits their ability to interact with each other, and/or antagonizes the neuronal disease-associated non-canonical activity of the GlyRS polypeptide. For instance, an antibody may bind to the GlyRS binding site that would otherwise interact with the cellular binding partner, and/or it may bind to the cellular binding partner's binding site that would otherwise interact with the GlyRS mutant. A cellular binding partner can be, for example, an intracellular, extracellular, or secreted protein (e.g., enzyme, transcription factor, cell surface receptor or soluble portion thereof, cytoskeletal protein), nucleic acid (RNA or DNA), lipid, and/or carbohydrate that specifically interacts with a disease-associated GlyRS mutant via one or more exposed neomorphic regions of the GlyRS mutant, preferably where that interaction is associated with a disease, such as a neuronal disease, including CMT diseases. Cellular binding partners may be intracellular or extracellular.

Specific examples of cellular binding partners include neuropilin transmembrane receptors, such as neuropilin-1 (NRP-1). Accordingly, certain antibodies may exhibit binding specificity for one or more neomorphic regions of GlyRS that specifically interact with a neuropilin such as NRP-1 and/or NRP-2, and certain antibodies may exhibit binding specificity for one or more regions of a neuropilin that specifically interact with a disease-associated GlyRS mutant, or a surface-exposed GlyRS neomorphic region. Certain of these and related antibodies may reduce or antagonize the GlyRS-mediated modulation of neuropilin transmembrane receptor activity, and may, for example, be characterized by their ability to competitively inhibit the interaction or binding between a disease-associated GlyRS mutant and a neuropilin transmembrane receptor, such as NRP-1. In particular embodiments, such antibodies do not (significantly) competitively inhibit the binding of neuropilins to one or more of their natural ligands. Accordingly, antibodies may be used to diagnose, treat, or prevent diseases, disorders or other conditions that are mediated by a mutant GlyRS polypeptide, such as by antagonizing its activity partially or fully.

An antibody, or antigen-binding fragment thereof, or other protein, is said to “exhibit binding specificity for,” “specifically bind to,” “immunologically bind to,” and/or is “immunologically reactive with” a selected mutant GlyRS polypeptide, and/or a neomorphic region thereof, if it reacts at a detectable level (within, for example, an ELISA assay) with the polypeptide, and does not react detectably in a statistically significant manner with unrelated polypeptides under similar conditions. In certain instances, an antibody does not significantly interact with a wild-type GlyRS polypeptide, preferably where that interaction is measured using a properly folded GlyRS, for example, a wild-type GlyRS in its native or most stable three-dimensional conformation in a cell.

Immunological binding, as used in this context, generally refers to the non-covalent interactions of the type which occur between an immunoglobulin molecule and an antigen for which the immunoglobulin is specific. The strength, or affinity of binding such as immunological binding interactions can be expressed in terms of the dissociation constant (K_{d}) of the interaction, wherein a smaller K_{d} represents a greater affinity. Immunological binding properties of selected polypeptides can be quantified using methods well known in the art. See, e.g., Davies et al. (1990) Annual Rev. Biochem. 59:439-473. In certain illustrative embodiments, an antibody has an affinity for a mutant GlyRS described herein, a neomorphic region described herein, or preferably an exposed neomorphic region of a disease-associated GlyRS mutant, of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM (including all integers and ranges in between). In certain embodiments, the affinity of the antibody for a disease-associated GlyRS mutant is stronger than its affinity for a wild-type GlyRS polypeptide, typically by about 1.5×, 2×, 2.5×, 3×, 3.5×, 4×, 4.5×, 5×, 6×, 7×, 8×, 9×, 10×, 15×, 20×, 25×, 30×, 40×, 50×, 60×, 70×, 80×, 90×, 100×, 200×, 300×, 400×, 500×, 600×, 700×, 800×, 900×, 1000× or more (including all integers and ranges in between). In certain embodiments, an antibody as an affinity for a wild-type GlyRS polypeptide of at least about (or no more than about) 0.05, 0.1, 0.25, 0.5, 0.75, 1, 2, 3, 4, 5, 6, 7, 8, 9, 10, 11, 12, 13, 14, 15, 16, 17, 18, 19, 20, 50, or 100 μM (including all integers and ranges in between). In certain embodiments, an antibody binds weakly or substantially undetectably to a wild-type GlyRS polypeptide in its native state in a cell.

Antibodies may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995. Particular examples include the VELOCIMMUNE® platform by REGENEREX® (see, e.g., U.S. Pat. No. 6,596,541). Antibodies can also be generated or identified by the use of phage display or yeast display libraries (see, e.g., U.S. Pat. No. 7,244,592; Chao et al., Nature Protocols. 1:755-768, 2006). Non-limiting examples of available libraries include cloned or synthetic libraries, such as the Human Combinatorial Antibody Library (HuCAL), in which the structural diversity of the human antibody repertoire is represented by seven heavy chain and seven light chain variable region genes. The combination of these genes gives rise to 49 frameworks in the master library. By superimposing highly variable genetic cassettes (CDRs=complementarity determining regions) on these frameworks, the vast human antibody repertoire can be reproduced. Also included are human libraries designed with human-donor-sourced fragments encoding a light-chain variable region, a heavy-chain CDR-3, synthetic DNA encoding diversity in heavy-chain CDR-1, and synthetic DNA encoding diversity in heavy-chain CDR-2. Other libraries suitable for use will be apparent to persons skilled in the art. The polypeptides of this invention may be used in the purification process in, for example, an affinity chromatography step.

The antigen binding site of an antibody is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Non-limiting examples of antibody fragments included within, but not limited to, the term “antigen-binding site” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., Nature 341:544-546, 1989), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., Science 242:423-426, 1988; and Huston et al., PNAS USA 85:5879-5883, 1988; and Osbourn et al., Nat. Biotechnol. 16:778, 1998). Such single chain antibodies are also intended to be encompassed within the term “antigen-binding portion” of an antibody. Any V_{H} and V_{L} sequences of specific scFv can be linked to human immunoglobulin constant region cDNA or genomic sequences, in order to generate expression vectors encoding complete IgG molecules or other isotypes. V_{H} and V_{L} can also be used in the generation of Fab, Fv or other fragments of immunoglobulins using either protein chemistry or recombinant DNA technology. Other forms of single chain antibodies, such as diabodies are also encompassed

An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. See, e.g., Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) PNAS USA. 85(16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

An “intrabody or fragment thereof” refers to antibodies that are expressed and function intracellularly. Intrabodies, in some embodiments, lack disulfide bonds and are capable of modulating the expression or activity of target genes through their specific binding activity. Intrabodies include single domain fragments such as isolated V_{H} and V_{L} domains and scFvs. An intrabody can include sub-cellular trafficking signals attached to the N or C terminus of the intrabodies to allow them to be expressed at high concentrations in the sub-cellular compartments where a target protein is located. Upon interaction with the target gene, an intrabody modulates target protein function, and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA or protein-RNA interactions. In one embodiment, an intrabody is a scFv

Each of the above-described molecules typically includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

Certain embodiments include single domain antibody (sdAbs or “nanobodies”), which refer to an antibody fragment consisting of a single monomeric variable antibody domain (see, e.g., U.S. Pat. Nos. 5,840,526; 5,874,541; 6,005,079, 6,765,087, 5,800,988; 5,874,541; and 6,015,695). Such sdABs typically have a molecular weight of about 12-15 kDa. In certain aspects, sdABs and other antibody molecules can be derived or isolated from the unique heavy-chain antibodies of immunized camels and llamas, often referred to as camelids. See, e.g., Conrath et al., JBC. 276:7346-7350, 2001.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients. See, e.g., U.S. Pat. Nos. 5,530,101; 5,585,089; 5,693,762; 6,180,370; and 7,022,500.

Similar to the other polypeptides of the invention, antibodies can also be pegylated to improve their pharmacokinetic properties. See, e.g., Veronese and Harris, Advanced Drug Delivery Reviews 54: 453-456, 2002, herein incorporated by reference.

The antibodies of the present invention can be used in any of the therapeutic, diagnostic, drug discovery, protein purification, and analytical methods and compositions described herein.

### Antibody Alternatives and Small Molecules

According to another aspect, the present invention further provides antibody alternatives, including “binding agents,” which are defined as proteins that bind with a similar specificity as an antibody, but lack any significant homology to an antigen-binding domain, and small molecules. Examples of binding agents include adnectins, anticalins, Darpins, anaphones, peptides, aptamers, etc., that exhibit binding specificity for at least one neomorphic region of human glycyl-tRNA synthetase (GlyRS), and/or to a fragment, variant or derivative thereof. Also included in the term “binding agents” are truncated or soluble forms of the cellular binding partner which are capable of binding to one or more neomorphic regions of GlyRS.

A “small molecule” refers to an organic compound that is of synthetic or biological origin (biomolecule), but is typically not a polymer. Binding agents and small molecules can be used in any of the therapeutic, diagnostic, drug discovery, or protein expression/purification, and analytical methods and compositions described herein. Biologic-based binding agents such as adnectins, avimers, and trinectins are particularly useful.

The binding characteristics of the binding agents and small molecules of the present invention can be similar to those of the antibodies described above. For instance, binding agents and small molecules of the present invention typically exhibit binding specificity for one or more GlyRS neomorphic regions described herein. These neomorphic regions typically show greater solvent exposure on the surface of disease-associated GlyRS mutants than on the surface of wild-type GlyRS. The binding agents and small molecules described herein thus exhibit binding specificity for one or more disease-associated GlyRS mutants.

In some embodiments, a binding agent or small molecule may exhibit binding specificity for both wild type human GlyRS and a disease-associated mutant human GlyRS; however, because the neomorphic regions described herein are typically not significantly solvent exposed in a properly folded wild-type GlyRS protein, the affinity of the binding agent for a given mutant human GlyRS is typically stronger than its affinity for wild type human GlyRS, for instance, by at least about 2× to at least about 100× or to at least about 1000× or more. In some embodiments the binding agent or small molecule is specific for the disease associated GlyRS mutant, and does not bind substantially to the wild type GlyRS.

Certain binding agents and small molecules of the present invention can thus distinguish between a disease-associated GlyRS mutant, such as CMT-associated GlyRS mutant, and a corresponding wild-type GlyRS, typically by binding with greater affinity to the mutant GlyRS (e.g., via one or more of its exposed neomorphic regions) than to the corresponding full-length GlyRS. For example, as noted above, binding agents and small molecules may exhibit binding specificity for one or more non-solvent exposed faces that are exposed in the GlyRS mutant but not in the full-length GlyRS. A binding agent or small molecule may thus exhibit binding specificity for a properly folded, disease-associated GlyRS mutant, preferably where the folding of that mutant is characterized, for example, by its native or most stable three-dimensional conformation in a cell or a physiological solution

Similar to antibodies, it should also be noted that a binding agent or small molecule may exhibit binding specificity for a denatured or partially denatured version of wild-type GlyRS, in part because the de-natured protein may allow access to the otherwise (relatively) hidden neomorphic regions. In specific embodiments, however, a binding agent does not show significant specific binding to a correctly folded version of wild-type GlyRS, where folding of that wild-type GlyRS is characterized, for example, by its native or most stable three-dimensional conformation in a cell or a physiological solution. Binding agents and small molecules may thus bind to unique three-dimensional structures that result from differences in folding between a given disease-associated GlyRS mutant and wild-type GlyRS. Such differences in folding may be localized (e.g., to a specific domain or region) or globalized. As one example, the folding of a mutant GlyRS may generate unique solvent-exposed, continuous or discontinuous epitopes that are not solvent-exposed in the wild-type human GlyRS.

Binding agents and small molecules may also exhibit binding specificity for peptides that consist or consist essentially of a GlyRS neomorphic region described herein, and/or a heterologous fusion protein comprising the same, e.g., one or more GlyRS neomorphic regions fused to one or more non-GlyRS polypeptide sequences. Examples of neomorphic regions or exposed faces include the region within A57-A663 and the “hot spot” regions defined by A57-A83, L129-D161, N208-Y320, V366-H378, P518-M531, L584-Y604, F620-R635, or D654-A663 of full-length GlyRS, and antigenic fragments of said regions. Also included are any combinations of these regions, e.g., A57-D161, A57-Y320, A57-H378, A57-M531, A57-Y604, A57-R635. These neomorphic “hot spot” regions are shared by a variety of disease-associated GlyRS mutants, including numerous CMT-associated GlyRS mutants. Additional examples of neomorphic regions include fragments of these “hot spots,” such as F79-A83, M227-L257, I232-N253, L258-R288, F147-K150, and E515-M531. Other neomorphic regions and their fragments are described, for example, in FIG. 2. For instance, regions characterized as “31-50%” or “>51%” can be included as neomorphic regions.

In certain embodiments, binding agents and small molecules of the present invention may exhibit binding specificity for a specific disease-associated GlyRS mutant, but not a different disease-associated GlyRS mutant. In some embodiments, the binding agents and small molecules of the present invention bind to a subset of the disease associated mutants, but not wild type GlyRS.

As noted above, even though disease-associated GlyRS mutants share a number of “hot spot” neomorphic regions, some neomorphic regions are relatively unique to, or have increased surface exposure in, a given disease-associated GlyRS mutant (see FIG. 2). Hence, certain antibodies may be used to treat and/or diagnose specific types of diseases, corresponding to specific GlyRS mutants. For example, the L129P mutant is associated with increased solvent exposure of amino acid residues A57-A83, G97-T110, E119-S178, N208-Y320, A326-N348, L361-H378, K423-E429, V461-Y464, L480-F486, K505-P554, V564-N570, L584-Y604, F620-I645, and D654-A663. As another example, the G240R mutant is associated with increased solvent exposure of residues A57-A83, G97-E123, F147-L189, F204-Y320, N348-H378, V461-Y464, K483-M531, D545-R642, and D654-E685. The G526R mutant is associated with increased solvent exposure of residues A57-A83, L129-K150, S183-V188, N208-Y320, N348-D389, K423-E429, L480-E485, D500-L511, P518-M531, T538-F550, L584-Y604, F620-I645, and D654-A663. The S581L mutant is associated with increased solvent exposure of residues A57-107, L129-D161, N208-Y320, V366-I402, K493-Q496, V513-M531, A555-R635, and D654-E685. The G598A mutant is associated with increased solvent exposure of residues A57-N106, L129-L203, N208-Y320, V366-D389, A421-Y464, E504-M531, F551-I645, and D654-A663. Examples of disease-associated GlyRS mutants include A57V, E71G, L129P, C157R, P234KY, G240R, P244L, I280F, H418R, D500N, G526R, S581L, and G598A mutants. See, e.g., FIG. 1A; and Nangle et al., PNAS USA. 104:11239-11244, 2007)

Binding agents and small molecules may also exhibit binding specificity for GlyRS mutants having a full or partial deletion of the WHEP domain. Without wishing to be bound by any one theory, the accompanying Examples suggest that deletion of the WHEP domain may induce a conformational change resembling that of the CMT-associated GlyRS mutations, exposing one or more neomorphic regions described herein. Similar to above, these and related binding agents and small molecules may be able to distinguish between a WHEP domain mutant of GlyRS and wild-type GlyRS, for example, by having significantly greater affinity for at least one exposed neomorphic region of a WHEP domain (deletion) mutant than for wild-type GlyRS; preferably, affinity comparisons are made between properly folded GlyRS proteins.

Because certain disease-associated GlyRS mutants lead to alteration of the dimer interface, and increase in monomer forms of GlyRS, certain binding agents and small molecules may exhibit binding specificity for the monomer but not the dimer form of human GlyRS. For certain binding agents and small molecules, their affinity for the monomer form of GlyRS is different than, for example, stronger than, their affinity for the dimer form of GlyRS by at least about 2× to at least about 100× or to at least about 1000×.

In certain embodiments, binding agents and small molecules are effective for modulating one or more of the non-canonical activities mediated by a disease-associated GlyRS mutant. A specific example of a non-canonical activity is modulation (inhibition or activation) of one or more neuropilin transmembrane receptors, such as NRP-1. Certain binding agents may reduce the GlyRS-mediated modulation (e.g., inhibition) of neuropilins. In some embodiments, for example, the binding agent is one that exhibits binding specificity for a disease-associated GlyRS mutant and/or its cellular binding partner, inhibits their ability to interact with each other, and/or antagonizes the non-canonical activity of the disease-associated GlyRS mutant. For instance, a binding agent or small molecule may bind to the GlyRS binding site that would otherwise interact with the cellular binding partner, and/or it may bind to the cellular binding partner's binding site that would otherwise interact with the GlyRS mutant. As noted above, a cellular binding partner can be a protein (e.g., enzyme, transcription factor, cell surface receptor or soluble portion thereof, cytoskeletal protein), nucleic acid (RNA or DNA), lipid, and/or carbohydrate that specifically interacts with a disease-associated GlyRS mutant via one or more exposed neomorphic regions of the GlyRS mutant, preferably where that interaction is associated with a disease, such as a neuronal disease, including CMT diseases. Cellular binding partners may be intracellular or extracellular.

Specific examples of cellular binding partners include neuropilin(s), including, for example, neuropilin transmembrane receptors, such as NRP-1 and NRP-2. Accordingly, certain binding agents and small molecules may exhibit binding specificity for one or more neomorphic regions of GlyRS that specifically interact with a neuropilin such as NRP-1 or NRP-2, and certain binding agents and small molecules may exhibit binding specificity for one or more regions of a neuropilin that specifically interact with a disease-associated GlyRS mutant. Certain of these and related binding agents and small molecules may reduce or antagonize the GlyRS-mediated modulation of neuropilin transmembrane receptors, and may, for example, be characterized by their ability to competitively inhibit the interaction or binding between a disease-associated GlyRS mutant and a neuropilin transmembrane receptor, such as NRP-1. Specific binding agents or small molecules do not (significantly) competitively inhibit the binding of neuropilins to one or more of their natural ligands. Accordingly, such binding agents and small molecules may be used to diagnose, treat, or prevent diseases, disorders or other conditions that are mediated by a disease-associated GlyRS mutant, such as by antagonizing or agonizing its activity partially or fully.

One specific class of binding agents includes naturally-occurring or engineered soluble isoforms of a neuropilin transmembrane receptor, such as any of the soluble isoforms disclosed in Table C. (see, e.g., Gagnon et al., PNAS. 97:2573-2578, 2000; Mamluk et al., Angiogenesis. 8:217-27, 2005). In certain embodiments, the soluble isoform at least comprises a portion of the extracellular domain of a neuropilin, such as NRP-1. One exemplary soluble isoform includes the 644-aa soluble NRP1 (sNRP1) isoform containing just the ayCUB and by-coagulation factor homology extracellular domains of NRP-1. Additional exemplary soluble isoforms include polypeptides having (approximately or precisely) residues Phe²²-Pro⁸⁵⁶, or residues Phe²²⁴-Lys⁶⁴⁴ of wild-type NRP1. Soluble isoforms of neuropilin may be engineered to optimize their pharmacological properties (e.g., Fc fusions, pegylation), as known in the art and described elsewhere herein for GlyRS polypeptides. Because these and other neuropilin soluble isoforms may interfere with the interaction between disease-associated GlyRS and membrane-bound neuropilins, such as by sequestering mutant GlyRS, and thus partially or fully antagonize the modulation of membrane-bound neuropilins by mutant GlyRS, they may be used to diagnose, treat, or prevent diseases, disorders or other conditions that are mediated by a disease-associated GlyRS mutant.

A binding agent or small molecule is said to “exhibit binding specificity for” or “specifically bind to” a neomorphic region of disease-associated GlyRS mutant or other mutant (e.g., WHEP domain mutant), and/or its cellular binding partner, if it reacts at a detectable level (within, for example, an ELISA assay) with the polypeptide or its cellular binding partner, and does not react delectably in a statistically significant manner with unrelated polypeptides under similar conditions. In certain instances, a binding agent does not significantly interact with a properly folded wild-type GlyRS, e.g., a wild-type GlyRS in its native or most stable three-dimensional conformation in a cell. In certain illustrative embodiments, a binding agent has an affinity for a neomorphic region of a disease-associated GlyRS mutant or other mutant (e.g., WHEP domain mutants) or its cellular binding partner of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28; 29, 30, 40, or 50 nM.

In certain embodiments, the affinity of the binding agent for a disease-associated GlyRS mutant is stronger than its affinity for a wild-type human GlyRS p (e.g., a properly folded wild-type GlyRS), typically by about 1.5×, 2×, 2.5×, 3×, 3.5×, 4×, 4.5×, 5×, 6×, 7×, 8×, 9×, 10×, 15×, 20×, 25×, 30×, 40×, 50×, 60×, 70×, 80×, 90×, 100×, 200×, 300×, 400×, 500×, 600×, 700×, 800×, 900×, 1000× or more (including all integers in between). In certain embodiments, a binding agent has an affinity for a corresponding wild-type GlyRS protein of at least about (or no more than about) 0.5, 1, 2, 3, 4, 5, 6, 7, 8, 9, 10, 11, 12, 13, 14, 15, 16, 17, 18, 19, 20, 50, or 100 μM. In certain embodiments, a binding agent binds weakly or substantially undetectably to a properly folded wild-type GlyRS polypeptide, for example, in its native three-dimensional conformation in a cell.

As noted above, “peptides” are included as binding agents. The term peptide typically refers to a polymer of amino acid residues and to variants and synthetic analogues of the same. In certain embodiments, the term “peptide” refers to relatively short polypeptides, including peptides that consist of about 3, 4, 5, 6, 7, 8, 9, 10, 11, 12, 13, 14, 15, 16, 17, 18, 19, 20, 25, 30, 31, 32, 33, 34, 35, 36, 37, 38, 39, 40, 41, 42, 43, 44, 45, 46, 47, 48, 49, 50 or more amino acids, including all integers and ranges (e.g., 5-10, 8-12, 10-15, 15-20, 20-25, 25-30, 30-40, 40-50) in between, and interact with a disease-associated GlyRS mutant via one or more of its exposed neomorphic regions. Peptides can be composed of naturally-occurring amino acids and/or non-naturally occurring amino acids.

As noted above, the present invention includes small molecules. A “small molecule” refers to an organic compound that is of synthetic or biological origin, but is typically not a polymer. Organic compounds include a large class of chemical compounds whose molecules contain carbon, typically excluding those that contain only carbonates, simple oxides of carbon, or cyanides. A “polymer” refers generally to a large molecule or macromolecule composed of repeating structural units, which are typically connected by covalent chemical bond. In certain embodiments, a small molecule has a molecular weight of less than 1000-2000 Daltons, typically between about 300 and 700 Daltons, and including about 50, 100, 150, 200, 250, 300, 350, 400, 450, 500, 550, 500, 650, 600, 750, 700, 850, 800, 950, 1000 or 2000 Daltons. Small molecule libraries are described elsewhere herein.

Aptamers are also included as binding agents (see, e.g., Ellington et al., Nature. 346, 818-22, 1990; and Tuerk et al., Science. 249, 505-10, 1990). Examples of aptamers included nucleic acid aptamers (e.g., DNA aptamers, RNA aptamers) and peptide aptamers. Nucleic acid aptamers refer generally to nucleic acid species that have been engineered through repeated rounds of in vitro selection or equivalent method, such as SELEX (systematic evolution of ligands by exponential enrichment), to bind to various molecular targets such as small molecules, proteins, nucleic acids, and even cells, tissues and organisms. See, e.g., U.S. Pat. Nos. 6,376,190; and 6,387,620 Hence, included are nucleic acid aptamers that bind to one or more neomorphic region(s) of a disease-associated GlyRS mutant described herein and/or their cellular binding partners.

Peptide aptamers typically include a variable peptide loop attached at both ends to a protein scaffold, a double structural constraint that typically increases the binding affinity of the peptide aptamer to levels comparable to that of an antibody's (e.g., in the nanomolar range). In certain embodiments, the variable loop length may be composed of about 10-20 amino acids (including all integers in between), and the scaffold may include any protein that has good solubility and compacity properties. Certain exemplary embodiments may utilize the bacterial protein Thioredoxin-A as a scaffold protein, the variable loop being inserted within the reducing active site (-Cys-Gly-Pro-Cys- loop in the wild protein), with the two cysteine lateral chains being able to form a disulfide bridge. Methods for identifying peptide aptamers are described, for example, in U.S. Application No. 2003/0108532. Hence, included are peptide aptamers that bind to the GlyRS neomorphic regions described herein and/or their cellular binding partners. Peptide aptamer selection can be performed using different systems known in the art, including the yeast two-hybrid system.

Also included are ADNECTINS™, AVIMERS™, and ANTICALINS that specifically bind to a GlyRS neomorphic region of the invention. ADNECTINS™ refer to a class of targeted biologics derived from human fibronectin, an abundant extracellular protein that naturally binds to other proteins. See, e.g., U.S. Application Nos. 2007/0082365; 2008/0139791; and 2008/0220049. ADNECTINS™ typically consists of a natural fibronectin backbone, as well as the multiple targeting domains of a specific portion of human fibronectin. The targeting domains can be engineered to enable an Adnectin™ to specifically recognize a therapeutic target of interest, such as a GlyRS neomorphic region of the invention, or a fragment thereof.

AVIMERS™ refer to multimeric binding proteins or peptides engineered using in vitro exon shuffling and phage display. Multiple binding domains are linked, resulting in greater affinity and specificity compared to single epitope immunoglobulin domains. See, e.g., Silverman et al., Nature Biotechnology. 23:1556-1561, 2005; U.S. Pat. No. 7,166,697; and U.S. Application Nos. 2004/0175756, 2005/0048512, 2005/0053973, 2005/0089932 and 2005/0221384.

Also included are designed ankyrin repeat proteins (DARPins), which include a class of non-immunoglobulin proteins that can offer advantages over antibodies for target binding in drug discovery and drug development. Among other uses, DARPins are ideally suited for in vivo imaging or delivery of toxins or other therapeutic payloads because of their favorable molecular properties, including small size and high stability. The low-cost production in bacteria and the rapid generation of many target-specific DARPins make the DARPin approach useful for drug discovery. Additionally, DARPins can be easily generated in multispecific formats, offering the potential to target an effector DARPin to a specific organ or to target multiple receptors with one molecule composed of several DARPins. See, e.g., Stumpp et al., Curr Opin Drug Discov Devel. 10:153-159, 2007; U.S. Application No. 2009/0082274; and PCT/EP2001/10454.

Certain embodiments include “monobodies,” which typically utilize the 10th fibronectin type III domain of human fibronectin (FNfn10) as a scaffold to display multiple surface loops for target binding. FNfn10 is a small (94 residues) protein with a β-sandwich structure similar to the immunoglobulin fold. It is highly stable without disulfide bonds or metal ions, and it can be expressed in the correctly folded form at a high level in bacteria. The FNfn10 scaffold is compatible with virtually any display technologies. See, e.g., Batori et al., Protein Eng. 15:1015-20, 2002; and Wojcik et al., Nat Struct Mol Biol., 2010; and U.S. Pat. No. 6,673,901.

Anticalins refer to a class of antibody mimetics, which are typically synthesized from human lipocalins, a family of binding proteins with a hypervariable loop region supported by a structurally rigid framework. See, e.g., U.S. Application No. 2006/0058510. Anticalins typically have a size of about 20 kDa. Anticalins can be characterized by a barrel structure formed by eight antiparallel β-strands (a stable β-barrel scaffold) that are pairwise connected by four peptide loops and an attached α-helix. In certain aspects, conformational deviations to achieve specific binding are made in the hypervariable loop region(s). See, e.g., Skerra, FEBS J. 275:2677-83, 2008, herein incorporated by reference.

The binding agents and small molecules of the present invention can be used in any of the therapeutic, diagnostic, drug discovery, protein purification, and analytical methods and compositions described herein.

### Polynucleotides

Embodiments of the present invention include polynucleotides that encode one or more of the polypeptides, peptides, fusion proteins, and/or antibodies described herein. Polynucleotides can be used in a variety of therapeutic, diagnostic, or protein production compositions and/or methods, as described herein and known in the art.

The term “polynucleotide” or “nucleic acid” as used herein designates mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA. The terms “DNA” and “polynucleotide” and “nucleic acid” refer to a DNA molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a GlyRS polypeptide. Included within the terms “DNA segment” and “polynucleotide” are DNA segments and smaller fragments of such segments, and also recombinant vectors, including, for example, plasmids, cosmids, phagemids, phage, viruses, and the like.

Additional coding or non-coding sequences may, but need not, be present within a polynucleotide of the present invention, and a polynucleotide may, but need not, be linked to other molecules and/or support materials. Hence, the polynucleotides of the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably.

It is therefore contemplated that a polynucleotide fragment of almost any length may be employed; with the total length preferably being limited by the ease of preparation and use in the intended recombinant DNA protocol. Included are polynucleotides of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 41, 43, 44, 45, 46, 47, 48, 49, 50, 60, 70, 80, 90, 100, 110, 120, 130, 140, 150, 160, 170, 180, 190, 200, 220, 240, 260, 270, 280, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000 or more (including all integers in between) bases in length, including any portion or fragment (e.g., greater than about 6, 7, 8, 9, or 10 nucleotides in length) of reference polynucleotide (e.g., base number X-Y, in which X is about 1-3000 or more and Y is about 10-3000 or more), or its complement.

Embodiments of the present invention also include “variants” of the polynucleotide sequences. Polynucleotide “variants” may contain one or more substitutions, additions, deletions and/or insertions in relation to a reference polynucleotide. Generally, variants of reference polynucleotide sequence may have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence (such as SEQ ID NO: 2) as determined by sequence alignment programs described elsewhere herein using default parameters. In certain embodiments, variants may differ from a reference sequence by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 41, 43, 44, 45, 46, 47, 48, 49, 50, 60, 70, 80, 90, 100 (including all integers in between) or more bases. In certain embodiments, such as when the polynucleotide variant encodes an polypeptide having a non-canonical activity, the desired activity of the encoded polypeptide is not substantially diminished relative to the unmodified polypeptide. The effect on the activity of the encoded polypeptide may generally be assessed as described herein.

Among other uses, these embodiments may be utilized to recombinantly produce a desired polypeptide (e.g., antibody) or variant thereof, or to express the polypeptide in a selected cell or subject. It will be appreciated by those of ordinary skill in the art that, as a result of the degeneracy of the genetic code, there are many nucleotide sequences that encode a polypeptide as described herein. Some of these polynucleotides may bear minimal homology to the nucleotide sequence of any native gene. Nonetheless, polynucleotides that vary due to differences in codon usage are specifically contemplated by the present invention, for example polynucleotides that are optimized for human and/or primate codon selection.

The polynucleotides of the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a polynucleotide fragment of almost any length may be employed; with the total length preferably being limited by the ease of preparation and use in the intended recombinant DNA protocol.

Polynucleotides and fusions thereof may be prepared, manipulated and/or expressed using any of a variety of well established techniques known and available in the art. For example, polynucleotide sequences which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence. Such polynucleotides are commonly referred to as “codon-optimized.” Any of the polynucleotides described herein may be utilized in a codon-optimized form. In certain embodiments, a polynucleotide can be codon optimized for use in specific bacteria such as E. coli or yeast such as S. cerevisiae (see, e.g., Burgess-Brown et al., Protein Expr Purif. 59:94-102, 2008).

Moreover, the polynucleotide sequences of the present invention can be engineered using methods generally known in the art in order to alter polypeptide encoding sequences for a variety of reasons, including but not limited to, alterations which modify the cloning, processing, expression and/or activity of the gene product. The polynucleotides of the present invention can be used in any of the therapeutic, diagnostic, research, or drug discovery, protein production compositions and methods described herein.

### Therapeutic Methods

Embodiments of the present invention include methods of treating a variety of diseases associated with the non-canonical activity of various disease-associated GlyRS mutants, including various neuronal diseases. Examples of neuronal disease that can be treated according to the present invention include distal spinal muscular atrophies (dSMA) and distal hereditary motor neuropathies (dHMN), such as Charcot-Marie-Tooth Disease Type 2D (CMT2D) and Distal Spinal Muscular Atrophy Type V (dSMA-V), among others described herein and known in the art. See, e.g., Sivakamur et al., Brain. 128:2304-2314, 2005; and Antonellis et al., Am. J. Hum. Genet. 72:1293-1299, 2003.

Other aspects include the treatment of diseases associated with aberrant activity of neuropilin-related pathways, for example, by relying on the ability of neomorphic regions of GlyRS to interfere with the interaction between neuropilins such as NRP-1 and one or more of their ligands, for example, where at least one neuropilin ligand is abnormally activating a neuropilin transmembrane receptor, or where neuropilin is over-expressed in a cell or tissue. As noted above, these embodiments relate to the discovery that certain disease-associated GlyRS mutants, such as CMT-associated GlyRS mutants, specifically interact with neuropilin transmembrane receptors such as neuropilin-1. Because neuropilins play diverse roles during the physiological regulation of processes such as angiogenesis, axon guidance, cell survival, migration, and invasion, the ability of exposed neomorphic regions of GlyRS to specifically interact with neuropilins suggests that GlyRS mutant polypeptides or other agents such as fusion proteins containing these neomorphic regions may have therapeutic utility in their own right, for example, where physiological problems occur due to aberrant activity of neuropilins, their ligands such as vascular endothelial growth factor (VEGF), placental growth factors (PGFs), and semaphorin family members (e.g., Sema3A), and/or other members of neuropilin-related pathways described herein and known in the art. In certain embodiments, such CMT-associated GlyRS mutants differ from SEQ ID NO: 1 by at least one mutation selected from the group consisting of A57V, E71G, L129P, C157R, P234KY, G240R, P244L, I280F, H418R, D500N, G526R, S581L, and G598A.

Distal spinal muscular atrophies (dSMA) and distal hereditary motor neuropathies (dHMNs) such as CMT represent a group of inherited disorders that affect the peripheral nerves (i.e., the nerves outside the brain and spine). These diseases can be characterized by slowly progressive muscle weakness and atrophy in the distal parts of the limbs caused, for example, by progressive anterior horn cell degeneration, among other processes. CMT Type 1 (CMT1) is a peripheral motor and sensory demyelinating neuropathy caused, for example, by degeneration of Schwann cells, and CMT Type 2 (CMT2) is typically characterized by primary axonal degeneration. CMT2 is often characterized by distal muscular atrophy, reduced compound motor action potentials, and/or reduced sensory nerve action potentials, but normal or mildly slowed motor nerve conduction velocity.

CMT disease is also known as known also as Morbus Charcot-Marie-Tooth, Charcot-Marie-Tooth neuropathy, hereditary motor and sensory neuropathy (HMSN), hereditary motor neuropathy type V (dHMN-V), hereditary sensor and motor neuropathy (HSMN), and peroneal muscular atrophy. Currently incurable, this family of diseases is one of the most common inherited neurological disorders, with an estimated 1 in 2500 individuals affected (see, e.g., Krajewski et al., Brain. 123:1516-27, 2000; and Skre, Clin. Genet. 6:98-118, 1974).

In certain embodiments, the subject to be treated has a disease that is associated with a particular GlyRS mutant. Examples of neuronal disease-associated GlyRS mutants include, without limitation, A57V, E71G, L129P, C157R, P234KY, G240R, P244L, I280F, H418R, D500N, G526R; S581L, and G598A of human GlyRS. See, e.g., Nangle et al., PNAS USA. 104:11239-11244, 2007; Park et al., PNAS USA. 105:11043-11049, 2008). Without being bound by any one theory, peripheral neuropathy in neuronal diseases such as CMT2 and dSMA-V may be caused by impaired GlyRS enzyme activity, GlyRS localization defects, or both (see, e.g., Antonellis et al., The Journal of Neuroscience. 26:10397-10406, 2006). However, GlyRS mutants also cause a dominant (e.g., axonal) form of neuronal disease in the human population, contributing, for example, to neurite distribution defects and axonal degeneration (see. e.g., Nangle et al., PNAS USA. 104:11239-11244, 2007; and Stum et al., Mol. Cell Neurosci. 46:432-443, 2011), which are believed to result from gain of function GlyRS mutants acquiring one or more specific interaction(s) not found in wild-type GlyRS.

Accordingly, included are methods of reducing or ameliorating a symptom of a neuronal disease, comprising administering to a subject an antibody or antigen-binding fragment described herein, a binding agent or small molecule described herein, and or a composition comprising the same, typically where said subject has a glycyl-tRNA synthetase (GlyRS) mutation associated with the neuronal disease. By exhibiting binding specificity for one or more specific neomorphic regions of GlyRS, which often show increased solvent exposure on the surface of disease-associated GlyRS mutants relative to wild-type GlyRS, such antibodies and binding agents can antagonize the dominant disease-associated non-canonical activity of one or more selected mutant GlyRS polypeptides, without significantly affecting the canonical activities of wild-type GlyRS. In specific embodiments, the binding agent is a soluble isoform of a neuropilin transmembrane receptor, such as a soluble isoform disclosed in Table C. In certain embodiments, the subject has a CMT disease such as CMT1 or CMT2 (including CMT2D), or dSMA-V, which is characterized by one or more GlyRS mutations described herein and known in the art, and the administration of an antibody, binding agent and/or small molecule reduces or ameliorates one or more symptoms of that disease.

Treatments can also be designed for subjects having specific GlyRS mutations, based, for example, on the pattern of exposed neomorphic regions characteristic to a given disease-associated GlyRS mutant. For instance, subjects having a L129P mutant of human GlyRS can be optionally treated with antibodies or other binding agents exhibiting binding specificity for one or more of the regions bounded by A57-A83, G97-T110, E119-S178, N208-Y320, A326-N348, L361-H378, K423-E429, V461-Y464, L480-F486, K505-P554, V564-N570, L584-Y604, F620-I645, or D654-A663 of the full-length L129P mutant of GlyRS. As another example, subjects having a G240R mutant of human GlyRS can be optionally treated with antibodies or other binding agents and small molecules exhibiting binding specificity for one or more of the regions bounded by A57-A83, G97-E123, F147-L189, F204-Y320, N348-H378, V461-Y464, K483-M531, D545-R642, or D654-E685 of the full-length G240R mutant of GlyRS. As another example, subjects having a G526R mutant can be optionally treated with antibodies or other binding agents and small molecules exhibiting binding specificity for one or more of the regions bounded by A57-A83, L129-K150, S183-V188, N208-Y320, N348-D389, K423-E429, L480-E485, D500-L511, P518-M531, T538-F550, L584-Y604, F620-I645, or D654-A663 of the full-length G526R mutant of GlyRS. Subjects having a S581L mutant can be optionally treated with antibodies or other binding agents and small molecules exhibiting binding specificity for one or more of the regions bounded by A57-107, L129-D161, N208-Y320, V366-I402, K493-Q496, V513-M531, A555-R635, D654-E685 of the full-length S581L mutant of GlyRS. Subjects having a G598A mutant can be optionally treated with antibodies or other binding agents and small molecules exhibiting binding specificity for one or more of the regions bounded by residues A57-N106, L129-L203, N208-Y320, V366-D389, A421-Y464, E504-M531, F551-I645, or D654-A663 of the full-length G598A mutant of GlyRS.

Further to the characteristics described above, exemplary clinical symptoms of neuronal diseases such as CMT include, without limitation, foot deformity (very high arch to feet), foot drop (inability to hold foot horizontal), loss of lower leg muscle, leading to skinny calves, numbness in the foot or leg, “slapping” gait (feet hit the floor hard when walking), and weakness of the hips, legs, or feet. These symptoms often present between mid-childhood and early adulthood. Later, similar symptoms may appear in the arms and hands, including, for example, claw-like hand deformities. Also included are complications such as a progressive inability to walk, a progressive muscle weakness, and injury to areas of the body that have decreased sensation. One specific symptom is reduced nerve conduction velocity. Accordingly, the antibodies, binding agents, and small molecules described herein may reduce or ameliorate any one or more of these characteristics, symptoms or complications of neuronal diseases such as dSMA and dHMN, including CMT disease such as CMT1 and CMT2 (including CMT2D).

Neuronal diseases such as CMT can be diagnosed according to routine techniques in the art. For example, physical examination may be used to observe signs such as difficulty lifting up the foot and making toe-out movements, lack of stretch reflexes in the legs, loss of muscle control and atrophy (shrinking of the muscles) in the foot or leg, and thickened nerve bundles under the skin of the legs. Muscle biopsies and/or nerve biopsies may be used to confirm a diagnosis, and nerve conduction tests can be used to tell the difference between different forms of the disease.

As noted above, certain embodiments relate to the treatment of diseases or conditions associated with aberrant activity or modulation of neuropilin-related pathways. Certain of these and related embodiments may utilize a polypeptide or other agent that comprises one or more exposed neomorphic regions of GlyRS, described elsewhere herein, which are capable of specifically binding to a neuropilin, and often competitively inhibit its interaction with one or more neuropilin ligands. These and related embodiments are typically used in situations where aberrant activity of neuropilin is not associated with the presence of mutant GlyRS polypeptides, but is rather independently associated with aberrant neuropilin activity or expression, and/or aberrant activity or expression of one or more neuropilin ligands.

Alternatively, as described elsewhere herein, antibodies, binding agents, and small molecules that exhibit binding specificity for neuropilins, and which competitively inhibit the binding of neuropilins to disease-associated GlyRS mutants, may be used in the treatment of disease resulting from the presence of disease-associated GlyRS mutants. Examples of such diseases include neuronal diseases such as distal spinal muscular atrophies (dSMA) and distal hereditary motor neuropathies (dHMN), described above. Specific examples include CMT diseases, such as CMT1 and CMT2 (including CMT2D). Accordingly, certain embodiments include methods of reducing or ameliorating a symptom of Charcot-Marie-Tooth Disease Type 2D or Distal Spinal Muscular Atrophy Type V (dSMA-V) disease, or other neuronal disease described herein, comprising administering to a subject an antibody or antigen-binding fragment, a binding agent, or a small molecule that exhibits binding specificity for a neuropilin, and which competitively inhibits binding between the neuropilin and a disease-associated GlyRS mutant, where said subject has GlyRS disease-associated mutation.

Neuropilins.

Neuropilins are typically single-pass transmembrane glycoprotein co-receptors with a large extracellular domain (ECD) and a short cytoplasmic domain that presents a PDZ binding site. Neuropilin-1 and neuropilin-2 are encoded respectively by the NRP1 and NRP2 genes (see, e.g., Soker et al., Cell. 92: 735-45, 1998; Chen et al., Neuron 19: 547-59, and Chen et al., J. Biol. Chem. 277: 24818-25, 1998). Multiple transcript variants encoding distinct isoforms have been identified for both genes, and exemplary neuropilins are disclosed in Table C.

TABLE C
Exemplary Neuropilins

| | | GenBank |
|---|---|---|
| | | Accession No |
| | | and SEQ. ID. |
| Name | Sequence | No. |
| neuropilin- | MERGLPLLCA VLALVLAPAG AFRNDECGDT IKIESPGYLT SPGYPHSYHP | NP_003864. |
| 1 isoform a | SEKCEWLIQA PDPYQRIMIN FNPHFDLEDR DCKYDYVEVF DGENENGHFR | 4 |
| | GKFCGKIAPP PVVSSGPFLF IKFVSDYETH GAGFSIRYEI FKRGPECSQN | SEQ. ID. |
| | YTTPSGVIKS PGFPEKYPNS LECTYIVFAP KMSEIILEFE SFDLEPDSNP | No. 4 |
| | PGGMFCRYDR LEIWDGFPDV GPHIGRYCGQ KTPGRIRSSS GILSMVFYTD | |
| | SAIAKEGFSA NYSVLQSSVS EDFKCMEALG MESGEIHSDQ ITASSQYSTN | |
| | WSAERSRLNY PENGWTPGED SYREWIQVDL GLLRFVTAVG TQGAISKETK | |
| | KKYYVKTYKI DVSSNGEDWI TIKEGNKPVL FQGNTNPTDV VVAVFPKPLI | |
| | TRFVRIKPAT WETGISMRFE VYGCKITDYP CSGMLGMVSG LISDSQITSS | |
| | NQGDRNWMPE NIRLVTSRSG WALPPAPHSY INEWLQIDLG EEKIVRGIII | |
| | QGGKHRENKV FMRKFKIGYS NNGSDWKMIM DDSKRKAKSF EGNNNYDTPE | |
| | LRTFPALSTR FIRIYPERAT HGGLGLRMEL LGCEVEAPTA GPTTPNGNLV | |
| | DECDDDQANC HSGTGDDFQL TGGTTVLATE KPTVIDSTIQ SEFPTYGFNC | |
| | EFGWGSHKTF CHWEHDNHVQ LKWSVLTSKT GPIQDHTGDG NFIYSQADEN | |
| | QKGKVARLVS PVVYSQNSAH CMTFWYHMSG SHVGTLRVKL RYQKPEEYDQ | |
| | LVWMAIGHQG DHWKEGRVLL HKSLKLYQVI FEGEIGKGNL GGIAVDDISI | |
| | NNHISQEDCA KPADLDKKNP EIKIDETGST PGYEGEGEGD KNISRKPGNV | |
| | LKTLEPILIT IIAMSALGVL LGAVCGVVLY CACWHNGMSE RNLSALENYN | |
| | FELVDGVKLK KDKLNTQSTY SEA | |
| neuropilin- | MERGLPLLCA VLALVLAPAG AFRNDKCGDT IKIESPGYLT SPGYPHSYHP | NP_0010197 |
| 1 isoform b | SEKCEWLIQA PDPYQRIMIN FNPHFDLEDR DCKYDYVEVF DGENENGHFR | 99.1 |
| | GKFCGKIAPP PVVSSGPFLF IKFVSDYETH GAGFSIRYEI FKRGPECSQN | SEQ. ID. |
| | YTTPSGVIKS PGFPEKYPNS LECTYIVFAP KMSEIILEFE SFDLEPDSNP | No. 5 |
| | PGGMFCRYDR LEIWDGFPDV GPHIGRYCGQ KTPGRIRSSS GILSMVFYTD | |
| | SAIAKEGFSA NYSVLQSSVS EDFKCMEALG MESGEIHSDQ ITASSQYSTN | |
| | WSAERSRLNY PENGWTPGED SYREWIQVDL GLLRFVTAVG TQGAISKETK | |
| | KKYYVKTYKI DVSSNGEDWI TIKEGNKPVL FQGNTNPTDV VVAVFPKPLI | |
| | TRFVRIKPAT WETGISMRFE VYGCKITDYP CSGMLGMVSG LISDSQITSS | |
| | NQGDRNWMPE NIRLVTSRSG WALPPAPHSY INEWLQIDLG EEKIVRGIII | |
| | QGGKHRENKV FMRKFKIGYS NNGSDWKMIM DDSKRKAKSF EGNNNYDTPE | |
| | LRTFPALSTR FIRIYPERAT HGGLGLRMEL LGCEVEAPTA GPTTPNGNLV | |
| | DECDDDQANC HSGTGDDFQL TGGTTVLATE KPTVIDSTIQ SGIK | |
| neuropilin- | MERGLPLLCA VLALVLAPAG AFRNDKCGDT IKIESPGYLT SPGYPHSYHP | NP_0010198 |
| 1 isoform c | SEKCEWLIQA PDPYQRIMIN FNPHFDLEDR DCKYDYVEVF DGENENGHFR | 00.1 |
| [Homo | GKFCGKIAPP PVVSSGPFLF IKFVSDYETH GAGFSIRYEI FKRGPECSQN | SEQ. ID. |
| sapiens] | YTTPSGVIKS PGFPEKYPNS LECTYIVFAP KMSEIILEFE SFDLEPDSNP | No. 6 |
| | PGGMFCRYDR LEIWDGFPDV GPHIGRYCGQ KTPGRIRSSS GILSMVFYTD | |
| | SAIAKEGFSA NYSVLQSSVS EDFKCMEALG MESGEIHSDQ ITASSQYSTN | |
| | WSAERSRLNY PENGWTPGED SYREWIQVDL GLLRFVTAVG TQGAISKETK | |
| | KKYYVKTYKI DVSSNGEDWI TIKEGNKPVL FQGNTNPTDV VVAVFPKPLI | |
| | TRFVRIKPAT WETGISMRFE VYGCKITDYP CSGMLGHVSG LISDSQITSS | |
| | NQGDRNWMPE NIRLVTSRSG WALPPAPHSY INEWLQIDLG EEKIVRGIII | |
| | QGGKHRENKV FMRKFKIGYS NNGSDWKMIM DDSKRKAKSF EGNNNYDTPE | |
| | LRTFPALSTR FIRIYPERAT HGGLGLRMEL LGCEVEGGTT VLATEKPTVI | |
| | DSTIQSGIK | |
| neuropilin- | MERGLPLLCA VLALVLAPAG AFRNDKCGDT IKIESPGYLT SPGYPHSYHP | AAG41406.1 |
| 1 soluble | SEKCEWLIQA PDPYQRIMIN FNPHFDLEDR DCKYDYVEVF DGENENGHFR | SEQ. ID. |
| isoform 11 | GKFCGKIAPP PVVSSGPFLF IKFVSDYETH GAGFSIRYEI FKRGPECSQN | No. 7 |
| [Homo | YTTPSGVIKS PGFPEKYPNS LECTYIVFAP KMSEIILEFE SFDLEPDSNP | |
| sapiens] | PGGMFCRYDR LEIWDGFPDV GPHIGRYCGQ KTPGRIRSSS GILSMVFYTD | |
| CRA_d | SAIAKEGFSA NYSVLQSSVS EDFKCMEALG MESGEIHSDQ ITASSQYSTN | |
| | WSAERSRLNY PENGWTPGED SYREWIQVDL GLLRFVTAVG TQGAISKETK | |
| | KKYYVKTYKI DVSSNGEDWI TIKEGNKPVL FQGNTNPTDV VVAVFPKPLI | |
| | TRFVRIKPAT WETGISMRFE VYGCKITDYP CSGMLGMVSG LISDSQITSS | |
| | NQGDRNWMPE NIRLVTSRSG WALPPAPHSY | |
| | INEWLQIDLG EEKIVRGIII QGGKHRENKV FMRKFKIGYS NNGSDWKMIM | |
| | DDSKRKAKSF EGNNNYDTPE LRTFPALSTR FIRIYPERAT HGGLGLRMEL | |
| | LGCEVEAPTA GPTTPNGNLV DECDDDQANC HSGTGDDFQL TGAETIFIPL | |
| | LYHFSSCLSW DQLTPVCVLV TPHGRELPRN RSCLARTRAS SEPHVIWIDE | |
| | LFLIATTICN NNLSHFESQR LGLS | |
| neuropilin- | MERGLPLLCA VLALVLAPAG AFRNDKCGDT IKIESPGYLT SPGYPHSYHP | AF145712 _1 |
| 1 soluble | SEKCEWLIQA PDPYQRIMIN FNPHFDLEDR DCKYDYVEVF DGENENGHFR | SEQ. ID. |
| isoform 12 | GKFCGKIAPP PVVSSGPFLF IKFVSDYETH GAGFSIRYEI FKRGPECSQN | No. 8 |
| [Homo | YTTPSGVIKS PGFPEKYPNS LECTYIVFAP KMSEIILEFE SFDLEPDSNP | |
| sapiens] | PGGMFCRYDR LEIWDGFPDV GPHIGRYCGQ KTPGRIRSSS GILSMVFYTD | |
| CRA_b | SAIAKEGFSA NYSVLQSSVS EDFKCMEALG HESGEIHSDQ ITASSQYSTN | |
| | WSAERSRLNY PENGWTPGED SYREWIQVDL GLLRFVTAVG TQGAISKETK | |
| | KKYYVKTYKI DVSSNGEDWI TIKEGNKPVL FQGNTNPTDV VVAVFPKPLI | |
| | TRFVRIKPAT WETGISMRFE VYGCKITDYP CSGMLGMVSG LISDSQITSS | |
| | NQGDRNWMPE NIRLVTSRSG WALPPAPHSY INEWLQIDLG EEKIVRGIII | |
| | QGGKHRENKV FMRKFKIGYS NNGSDWKMIM DDSKRKAKSF EGNNNYDTPE | |
| | LRTFPALSTR FIRIYPERAT HGGLGLRMEL LGCEVEAPTA GPTTPNGNLV | |
| | DECDDDQANC HSGTGDDFQL TGGTTVLATE KPTVIDSTIQ SGIK | |
| neuropilin | MERGLPLLCA VLALVLAPAG AFRNDKCGDT IKIESPGYLT SPGYPHSYHP | EAW85942.1 |
| 1, isoform | SEKCEWLIQA PDPYQRIMIN FNPHFDLEDR DCKYDYVEVF DGENENGHFR | SEQ. ID. |
| CRA_a | GKFCGKIAPP PVVSSGPFLF IKFVSDYETH GAGFSIRYEI FKRGPECSQN | No. 9 |
| | YTTPSGVIKS PGFPEKYPNS LECTYIVFAP KMSEIILEFE SFDLEPDSNP | |
| | PGGMFCRYDR LEIWDGFPDV GPHIGRYCGQ KTPGRIRSSS GILSMVFYTD | |
| | SAIAKEGFSA NYSVLQSSVS EDFKCMEALG MESGEIHSDQ ITASSQYSTN | |
| | WSAERSRLNY PENGWTPGED SYREWIQVDL GLLRFVTAVG TQGAISKETK | |
| | KKYYVKTYKI DVSSNGEDWI TIKEGNKPVL FQGNTNPTDV VVAVFPKPLI | |
| | TRFVRIKPAT WETGISMRFE VYGCKITDYP CSGMLGMVSG LISDSQITSS | |
| | NQGDRNWMPE NIRLVTSRSG WALPPAPHSY INEWLQIDLG EEKIVRGIII | |
| | QGGKHRENKV FMRKFKIGYS NNGSDWKMIM DDSKRKAKSF EGNNNYDTPE | |
| | LRTFPALSTR FIRIYPERAT HGGLGLRMEL LGCEVEGGTT VLATEKPTVI | |
| | DSTIQSGIK | |
| NRP2a | MDMFPLTWVFLALYFSRHQVRGQPDPPCGGRLNSKDAGYITSPGYPQDYPSHQNCEW | NM_003872. |
| | IVYAPEPNQKIVLNENPHFEIEKHDCKYDFIEIRDGDSESADLLGKHCGNIAPPTII | 2 |
| | SSGSMLYIKFTSDYARQGAGFSLRYEIFKTGSEDCSKNFTSPNGTIESPGFPEKYPH | SEQ. ID. |
| | NLDCTFTILAKPKMEIILQFLIFDLEHDPLQVGEGDCKYDWLDIWDGIPHVGPLIGK | No. 10 |
| | YCGTKTPSELRSSTGILSLTFHTDMAVAKDGESARYYLVHQEPLENFQCNVPLGMES | |
| | GRIANEQISASSTYSDGRWTPQQSRLHGDDNGWTPNLDSNKEYLFLTMLTAIATQGA | |
| | ISRETQNGYYVKSYKLEVSTNGEDWMVYRHGKNHKVFQANNDATEVVLNKLHAPLLT | |
| | RFVRIRPQTWHSGIALRLELFGCRVTDAPCSNMLGMLSGLIADSQISASSTQEYLWS | |
| | PSAARLVSSRSGWFPRIPQAQPGEEWLQVDLGTPKTVKGVIIQGARGGDSITAVEAR | |
| | AFVRKEKVSYSLNGKDWEYIQDPRTQQPKLFEGNMHYDTPDIRRFDPIPAQYVRVYP | |
| | ERWSPAGIGMRLEVLGCDWTDSKPTVETLGPTVKSEETTTPYPTEEEATECGENCSF | |
| | EDDKDLQLPSGFNCNFDFLEEPCGWMYDHAKWLRTTWASSSSPNDRTFPDDRNFLRL | |
| | QSDSQREGQYARLISPPVHLPRSPVCMEFQYQATGGRGVALQVVREASQESKLLWVI | |
| | REDQGGEWKHGRIILPSYDMEYQIVFEGVIGKGRSGEIAIDDIRISTDVPLENCMEP | |
| | ISAFAVDIPEIHEREGYEDEIDDEYEVDWSNSSSATSGSGAPSTDKEKSWLYTLDPI | |
| | LITIIAMSSLGVLLGATCAGLLLYCTCSYSGLSSRSCTTLENYNFELYDGLKHKVKM | |
| | NHQKCCSEA” | |
| neuropilin- | MDMFPLTWVF LALYFSRHQV RGQPDPPCGG RLNSKDAGYI TSPGYPQDYP | NP_958936. |
| 2 isoform 3 | SHQNCEWIVY APEPNQKIVL NFNPHFEIEK HDCKYDFIEI RDGDSESADL | 1 |
| precursor | LGKHCGNIAP PTIISSGSML YIKFTSDYAR QGAGFSLRYE IFKTGSEDCS | SEQ. ID. |
| [Homo | KNFTSPNGTI ESPGFPEKYP HNLDCTFTIL AKPKMEIILQ FLIFDLEHDP | No. 11 |
| sapiens] | LQVGEGDCKY DWLDIWDGIP HVGPLIGKYC GTKTPSELRS STGILSLTFH | |
| | TDMAVAKDGF SARYYLVHQE PLENFQCNVP LGMESGRIAN EQISASSTYS | |
| | DGRWTPQQSR LHGDDNGWTP NLDSNKEYLQ VDLRFLTMLT AIATQGAISR | |
| | ETQNGYYVKS YKLEVSTNGE DWNVYRHGKN HKVFQANNDA TEVVLNKLHA | |
| | PLLTRFVRIR PQTWHSGIAL RLELFGCRVT DAPCSNMLGM LSGLIADSQI | |
| | SASSTQEYLW SPSAARLVSS RSGWFPRIPQ AQPGEEWLQV DLGTPKTVKG | |
| | VIIQGARGGD SITAVEARAF VRKFKVSYSL NGKDWEYIQD PRTQQPKLFE | |
| | GNMHYDTPDI RRFDPIPAQY VRVYPERWSP AGIGMRLEVL GCDWTDSKPT | |
| | VETLGPTVKS EETTTPYPTE EEATECGENC SFEDDKDLQL PSGFNCNFDF | |
| | LEEPCGWMYD HAKWLRTTWA SSSSPNDRTF PDDRNFLRLQ SDSQREGQYA | |
| | RLISPPVHLP RSPVCMEFQY QATGGRGVAL QVVREASQES KLLWVIREDQ | |
| | GGEWKHGRII LPSYDMEYQI VFEGVIGKGR SGEIAIDDIR ISTDVPLENC | |
| | MEPISAFADE YEVDWSNSSS ATSGSGAPST DKEKSWLYTL DPILITIIAM | |
| | SSLGVLLGAT CAGLLLYCTC SYSGLSSRSC TTLENYNFEL YDGLKHKVKM | |
| | NHQKCCSEA | |
| neuropilin- | MDMFPLTWVF LALYFSRHQV RGQPDPPCGG RLNSKDAGYI TSPGYPQDYP | NP_957719. |
| 2 isoform 5 | SHQNCEWIVY APEPNQKIVL NFNPHFEIEK HDCKYDFIEI RDGDSESADL | 1 |
| precursor | LGKHCGNIAP PTIISSGSML YIKFTSDYAR QGAGFSLRYE IFKTGSEDCS | SEQ. ID. |
| [Homo | KNFTSPNGTI ESPGFPEKYP HNLDCTFTIL AKPKMEIILQ FLIFDLEHDP | No. 12 |
| sapiens] | LQVGEGDCKY DWLDIWDGIP HVGPLIGKYC GTKTPSELRS STGILSLTFH | |
| | TDMAVAKDGF SARYYLVHQE PLENFQCNVP LGMESGRIAN EQISASSTYS | |
| | DGRWTPQQSR LHGDDNGWTP NLDSNKEYLQ VDLRFLTMLT AIATQGAISP | |
| | ETQNGYYVKS YKLEVSTNGE DWMVYRHGKN HKVFQANNDA TEVVLNKLHA | |
| | PLLTRFVRIR PQTWHSGIAL RLELFGCRVT DAPCSNMLGM LSGLIADSQI | |
| | SASSTQEYLW SPSAARLVSS RSGWFPRIPQ AQPGEEWLQV DLGTPKTVKG | |
| | VIIQGARGGD SITAVEARAF VRKFKVSYSL NGKDWEYIQD PRTQQPKLFE | |
| | GNMHYDTPDI RRFDPIPAQY VRVYPERWSP AGIGMRLEVL GCDWTDSKPT | |
| | VETLGPTVKS EETTTPYPTE EEATECGENC SFEDDKDLQL PSGFNCNFDF | |
| | LEEPCGWMYD HAKWLRTTWA SSSSPNDRTF PDDRNFLRLQ SDSQREGQYA | |
| | RLISPPVHLP RSPVCMEFQY QATGGRGVAL QVVREASQES KLLWVIREDQ | |
| | GGEWKHGRII LPSYDMEYQI VFEGVIGKGR SGEIAIDDIR ISTDVPLENC | |
| | MEPISAFAGG TLLPGTEPTV DTVPMQPIPA YWYYVMAAGG AVLVLVSVAL | |
| | ALVLHYHRFR YAAKKTDHSI TYKTSHYTNG APLAVEPTLT IKLEQDRGSH | |
| | C | |
| neuropilin- | MDMFPLTWVF LALYFSRHQV RGQPDPPCGG RLNSKDAGYI TSPGYPQDYP | NP_957713. |
| 2 isoform 1 | SHQNCEWIVY APEPNQKIVL NFNPHFEIEK HDCKYDFIEI RDGDSESADL | 1 |
| precursor | LGKHCGNIAP PTIISSGSML YIKFTSDYAR QGAGFSLRYE IFKTGSEDCS | SEQ. ID. |
| [Homo | KNFTSPNGTI ESPGFPEKYP HNLDCTFTIL AKPKMEIILQ FLIFDLEHDP | No. 13 |
| sapiens] | LQVGEGDCKY DWLDIWDGIP HVGPLIGKYC GTKTPSELRSSTGILSLTFH | |
| | TDMAVAKDGF SARYYLVHQE PLENFQCNVP LGMESGRIAN EQISASSTYS | |
| | DGRWTPQQSR LHGDDNGWTP NLDSNKEYLQ VDLRFLTMLT AIATQGAISR | |
| | ETQNGYYVKS YKLEVSTNGE DWMVYRHGKN HKVFQANNDA TEVVLNKLHA | |
| | PLLTRFVRIR PQTWHSGIAL RLELFGCRVT DAPCSNMLGM LSGLIADSQI | |
| | SASSTQEYLW SPSAARLVSS RSGWFPRIPQ AQPGEEWLQV DLGTPKTVKG | |
| | VIIQGARGGD SITAVEARAF VRKFKVSYSL NGKDWEYIQD PRTQQPKLFE | |
| | GNMHYDTPDI RRFDPIPAQY VRVYPERWSP AGIGMRLEVL GCDWTDSKPT | |
| | VETLGPTVKS EETTTPYPTE EEATECGENC SFEDDKDLQL PSGFNCNFDF | |
| | LEEPCGWMYD HAKWLRTTWA SSSSPNDRTF PDDRNFLRLQ SDSQREGQYA | |
| | RLISPPVHLP RSPVCMEFQY QATGGRGVAL QVVREASQES KLLWVIREDQ | |
| | GGEWKHGRII LPSYDMEYQI VFEGVIGKGR SGEIAIDDIR ISTDVPLENC | |
| | MEPISAFAGE NFKVDIPEIH EREGYEDEID DEYEVDWSNS SSATSGSGAP | |
| | STDKEKSWLY TLDPILITII AMSSLGVLLG ATCAGLLLYC TCSYSGLSSR | |
| | SCTTLENYNF ELYDGLKHKV KMNHQKCCSE A | |
| neuropilin- | MDMFPLTWVF LALYFSRHQV RGQPDPPCGG RLNSKDAGYI TSPGYPQDYP | NP_957716. |
| 2 isoform 6 | SHQNCEWIVY APEPNQKIVL NFNPHFEIEK HDCKYDFIEI RDGDSESADL | 1 |
| precursor | LGKHCGNIAP PTIISSGSML YIKFTSDYAR QGAGFSLRYE IFKTGSEDCS | SEQ. ID. |
| [Homo | KNFTSPNGTI ESPGFPEKYP HNLDCTFTIL AKPKMEIILQ FLIFDLEHDP | No. 14 |
| sapiens] | LQVGEGDCKY DWLDIWDGIP HVGPLIGKYC GTKTPSELRS STGILSLTFH | |
| | TDMAVAKDGF SARYYLVHQE PLENFQCNVP LGMESGRIAN EQISASSTYS | |
| | DGRWTPQQSR LHGDDNGWTP NLDSNKEYLQ VDLRFLTMLT AIATQGAISR | |
| | ETQNGYYVKS YKLEVSTNGE DWMVYRHGKN HKVFQANNDA TEVVLNKLHA | |
| | PLLTRFVRIR PQTWHSGIAL RLELFGCRVT DAPCSNMLGM LSGLIADSQI | |
| | SASSTQEYLW SPSAARLVSS RSGWFPRIPQ AQPGEEWLQV DLGTPKTVKG | |
| | VIIQGARGGD SITAVEARAF VRKFKVSYSL NGKDWEYIQD PRTQQPKVGC | |
| | SWRPL | |
| neuropilin- | MDMFPLTWVF LALYFSRHQV RGQPDPPCGG RLNSKDAGYI TSPGYPQDYP | NP_061004. |
| 2 isoform 4 | SHQNCEWIVY APEPNQKIVL NFNPHFEIEK HDCKYDFIEI RDGDSESADL | 3 |
| precursor | LGKHCGNIAP PTIISSGSML YIKFTSDYAR QGAGFSLRYE IFKTGSEDCS | SEQ. ID. |
| [Homo | KNFTSPNGTI ESPGFPEKYP HNLDCTFTIL AKPKMEIILQ FLIFDLEHDP | No. 15 |
| sapiens] | LQVGEGDCKY DWLDIWDGIP HVGPLIGKYC GTKTPSELRS STGILSLTFH | |
| | TDMAVAKDGF SARYYLVHQE PLENFQCNVP LGMESGRIAN EQISASSTYS | |
| | DGRWTPQQSR LHGDDNGWTP NLDSNKEYLQ VDLRFLTMLT AIATQGAISR | |
| | ETQNGYYVKS YKLEVSTNGE DWMVYRHGKN HKVFQANNDA TEVVLNKLHA | |
| | PLLTRFVRIR PQTWHSGIAL RLELFGCRVT DAPCSNMLGM LSGLIADSQI | |
| | SASSTQEYLW SPSAARLVSS RSGWFPRIPQ AQPGEEWLQV DLGTPKTVKG | |
| | VIIQGARGGD SITAVEARAF VRKFKVSYSL NGKDWEYIQD PRTQQPKLFE | |
| | GNMHYDTPDI RRFDPIPAQY VRVYPERWSP AGIGMRLEVL GCDWTDSKPT | |
| | VETLGPTVKS EETTTPYPTE EEATECGENC SFEDDKDLQL PSGFNCNFDF | |
| | LEEPCGWMYD HAKWLRTTWA SSSSPNDRTF PDDRNFLRLQ SDSQREGQYA | |
| | RLISPPVHLP RSPVCMEFQY QATGGRGVAL QVVREASQES KLLWVIREDQ | |
| | GGEWKHGRII LPSYDMEYQI VFEGVIGKGR SGEIAIDDIR ISTDVPLENC | |
| | MEPISAFAGE NFKGGTLLPG TEPTVDTVPM QPIPAYWYYV MAAGGAVLVL | |
| | VSVALALVLH YHRFRYAAKK TDHSITYKTS HYTNGAPLAV EPTLTIKLEQ | |
| | DRGSHC | |
| neuropilin- | MDMFPLTWVF LALYFSRHQV RGQPDPPCGG RLNSKDAGYI TSPGYPQDYP | NP_003863. |
| 2 isoform 2 | SHQNCEWIVY APEPNQKIVL NFNPHFEIEK HDCKYDFIEI RDGDSESADL | 2 |
| precursor | LGKHCGNIAP PTIISSGSML YIKFTSDYAR QGAGFSLRYE IFKTGSEDCS | SEQ. ID. |
| [Homo | KNFTSPNGTI ESPGFPEKYP HNLDCTFTIL AKPKMEIILQ FLIFDLEHDP | No. 16 |
| sapiens] | LQVGEGDCKY DWLDIWDGIP HVGPLIGKYC GTKTPSELRS STGILSLTFH | |
| | TDMAVAKDGF SARYYLVHQE PLENFQCNVP LGMESGRIAN EQISASSTYS | |
| | DGRWTPQQSR LHGDDNGWTP NLDSNKEYLQ VDLRFLTMLT AIATQGAISR | |
| | ETQNGYYVKS YKLEVSTNGE DWHVYRHGKN HKVFQANNDA TEVVLNKLHA | |
| | PLLTRFVRIR PQTWHSGIAL RLELFGCRVT DAPCSNMLGM LSGLIADSQI | |
| | SASSTQEYLW SPSAARLVSS RSGWFPRIPQ AQPGEEWLQV DLGTPKTVKG | |
| | VIIQGARGGD SITAVEARAF VRKFKVSYSL NGKDWEYIQD PRTQQPKLFE | |
| | GNMHYDTPDI RRFDPIPAQY VRVYPERWSP AGIGMRLEVL GCDWTDSKPT | |
| | VETLGPTVKS EETTTPYPTE EEATECGENC SFEDDKDLQL PSGFNCNFDF | |
| | LEEPCGWMYD HAKWLRTTWA SSSSPNDRTF PDDRNFLRLQ SDSQREGQYA | |
| | RLISPPVHLP RSPVCMEFQY QATGGRGVAL QVVREASQES KLLWVIREDQ | |
| | GGEWKHGRII LPSYDMEYQI VFEGVIGKGR SGEIAIDDIR ISTDVPLENC | |
| | MEPISAFAVD IPEIHEREGY EDEIDDEYEV DWSNSSSATS GSGAPSTDKE | |
| | KSWLYTLDPI LITIIAMSSL GVLLGATCAG LLLYCTCSYS GLSSRSCTTL | |
| | ENYNFELYDG LKHKVKMNHQ KCCSEA | |
| neuropilin- | MDMFPLTWVF LALYFSRHQV RGQPDPPCGG RLNSKDAGYI TSPGYPQDYP | AAG41405.1 |
| 2 soluble | SHQNCEWIVY APEPNQKIVL NFNPHFEIEK HDCKYDFIEI RDGDSESADL | SEQ. ID. |
| isoform 9 | LGKHCGNIAP PTIISSGSML YIKFTSDYAR QGAGFSLRYE IFKTGSEDCS | No. 17 |
| [Homo | KNFTSPNGTI ESPGFPEKYP HNLDCTFTIL AKPKMEIILQ FLIFDLEHDP | |
| sapiens] | LQVGEGDCKY DWLDIWDGIP HVGPLIGKYC GTKTPSELRS STGILSLTFH | |
| | TDMAVAKDGF SARYYLVHQE PLENFQCNVP LGMESGRIAN EQISASSTYS | |
| | DGRWTPQQSR LHGDDNGWTP NLDSNKEYLQ VDLRFLTMLT AIATQGAISR | |
| | ETQNGYYVKS YKLEVSTNGE DWMVYRHGKN HKVFQANNDA TEVVLNKLHA | |
| | PLLTRFVRIR PQTWHSGIAL RLELFGCRVT DAPCSNMLGM LSGLIADSQI | |
| | SASSTQEYLW SPSAARLVSS RSGWFPRIFQ AQPGEEWLQV DLGTPKTVKG | |
| | VIIQGARGGD SITAVEARAF VRKFKVSYSL NGKDWEYIQD PRTQQPKVGC | |
| | SWRPL | |

The neuropilin may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, substitutions. Naturally-occurring chemical modifications including post-translational modifications and degradation products of neuropilin, are also specifically included in any of the methods of the invention including for example, pyroglutamyl, iso-aspartyl, proteolytic, phosphorylated, glycosylated, reduced, oxidatized, isomerized, and deaminated variants of neuropilin.

The neuropilins which may be used in any of the methods of the invention may have amino acid sequences which are substantially homologous, or substantially similar to any of the native neuropilin amino acid sequences, for example, to any of the native neuropilin gene sequences listed in Table C. In some aspects, the neuropilin may have an amino acid sequence having at least 30% preferably at least 40, 50, 60, 70, 75, 80, 85, 90, 95, 98, or 99% identity with a neuropilin listed in Table C.

It is known in the art to synthetically modify the sequences of proteins or peptides, while retaining their useful activity, and this may be achieved using techniques which are standard in the art and widely described in the literature, e.g., random or site-directed mutagenesis, cleavage, and ligation of nucleic acids, or via the chemical synthesis or modification of amino acids or polypeptide chains. For instance, conservative amino acid mutations changes can be introduced into neuropilin and are considered within the scope of the invention.

The neuropilin amino acid sequence may thus include one or more amino acid deletions, additions, insertions, and/or substitutions based on any of the naturally-occurring isoforms of neuropilin gene. These may be contiguous or non-contiguous. Representative variants may include those having 1 to 10, or more preferably 1 to 4, 1 to 3, or 1 or 2 amino acid substitutions, insertions, and/or deletions as compared to any of sequences listed in Table C. In some embodiments soluble forms of neuropilin are known, and such deletion mutants, including for example SEQ ID NOs:7, 8, 9 and 17 from Table C, are preferred for some methods as disclosed herein. In certain embodiments soluble forms of neuropilin may be readily prepared by deletion of the c-terminal transmembrane domain.

The present invention also contemplates the use of neuropilin chimeric or fusion proteins. As used herein, a “chimeric protein” or “fusion protein” includes a neuropilin polypeptide linked to either another neuropilin polypeptide (e.g., to create multiple fragments), to a heterologous non neuropilin polypeptide, or to both. A “heterologous polypeptide” refers to a polypeptide having an amino acid sequence corresponding to a protein which is different from a human neuropilin sequence, and which can be derived from the same or a different organism. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

The fusion partner may be designed and included for essentially any desired purpose provided they do not adversely affect the activity of the neuropilin. For example, in one embodiment, a fusion partner may comprise a sequence that assists in expressing the protein (an expression enhancer) at higher yields than the native recombinant protein. Other fusion partners may be selected so as to increase the solubility of the protein or to enable the protein to be targeted to desired intracellular compartments

The fusion protein can include a moiety which has a high affinity for a ligand. For example, the fusion protein can be a GST-neuropilin fusion protein in which the neuropilin sequences are fused to the C-terminus of the GST sequences. As another example, a neuropilin peptide or polypeptide may be fused to an affinity/and or epitope tag at the C-terminus, such as a poly-histidine tag such as a sequence comprising the sequence L-E-H-H-H-H-H-H (SEQ ID NO:3). Such fusion proteins can facilitate the purification and/or identification of a neuropilin polypeptide. Selected moieties can also be used to attach the fusion protein to a surface, for example, to facilitate screening for agents that specifically bind to the neuropilin polypeptide or peptide. Alternatively, the fusion protein can be a neuropilin protein containing a heterologous signal sequence at its N-terminus. In certain host cells, expression and/or secretion of neuropilin proteins can be increased through use of a heterologous signal sequence.

More generally, fusion to heterologous sequences, such as an Fc fragment, may be utilized to remove unwanted characteristics or to improve the desired characteristics (e.g., pharmacokinetic properties) of a neuropilin polypeptide. For example, fusion to a heterologous sequence may increase chemical stability, decrease immunogenicity, improve in vivo targeting, and/or increase half-life in circulation of a neuropilin polypeptide.

Fusion proteins may generally be prepared using standard techniques. For example, DNA sequences encoding the polypeptide components of a desired fusion may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures, if desired. Such a peptide linker sequence is incorporated into the fusion protein using standard techniques well known in the art. Certain peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The variants, derivatives, and fusion proteins of neuropilin are functionally equivalent in that they have detectable neuropilin activity. More particularly, they exhibit at least 5%, at least 10%, at least 20%, at least 30%, at least 40%, preferably at least 60%, more preferably at least 80% of the activity of human neuropilin gene, and are thus they are capable of substituting for human neuropilin itself.

Such activity means any activity exhibited by a native neuropilin, whether a physiological response exhibited in an in vivo or in vitro test system, or any biological activity or reaction mediated by a native neuropilin, e.g., in an enzyme, or cell based assay. In one example, such an activity includes the ability for the neuropilin to bind to mutant GlyRS, but not the wild type GlyRS, as more fully described in the examples. All such variants, derivatives, fusion proteins, or fragments of the neuropilin are included, and may be used in any of the polynucleotides, vectors, host cell and methods disclosed herein, and are subsumed under the terms “neuropilin(s) or neuropilin polypeptide.”

Accordingly the term “neuropilin(s)” includes any naturally-occurring and synthetic forms of neuropilin that retain neuropilin activity. Exemplary naturally occurring alternatively spliced neuropilin transcripts are disclosed in Table C. All such naturally-occurring isoforms of neuropilin (SEQ ID NO: 4-17) are included in any of the methods and compositions of the invention, as long as they retain the ability to bind to a mutant GlyRS.

The term “neuropilin transmembrane receptor” refers to any of the membrane associated forms of neuropilin, including for example any of the transmembrane forms of neuropilin listed in Table C, and any co-receptors thereof with other proteins that bind to a neuropilin ligand. The term “soluble isoform of a neuropilin transmembrane receptor” or “soluble neuropilin” refers to any naturally occurring, or engineered (e.g., truncated) forms of neuropilin which lack an intact transmembrane domain, and are not primarily (e.g., less than 50%) associated with the cell membrane. Exemplary naturally occurring soluble neuropilins include any of the soluble forms of neuropilin listed in Table C. In one aspect such soluble neuropilins are selected from SEQ ID NO: 7, 8, 9 or 17.

The basic structure of neuropilins typically comprises at least five domains: three extracellular domains (a1a2, b1b2, and, c), a transmembrane domain, and a short cytoplasmic domain. The a1a2 domain is a CUB domain (named for its identification in complement components C1r and C1s, Uegf, and bmp1), a domain commonly found in developmentally regulated proteins and which generally contains four cysteine residues that make two disulfide bridges. The neuropilin CUB domain shares homology with complement components C1r and C1s. The first two extracellular domains of NRP-1 (a1a2 and b1b2) bind ligand. Additionally, the structure-function studies using neuropilin mutants containing deletions within the “a” and “b” domains show that the CUB domains (a1a2 and b1b2) are required for semaphorin binding. The third extracellular domain is critical for homo-dimerization or hetero-dimerization. The structure of the B1 domain (coagulation factor 5/8 type) of neuropilin-1 was determined through X-Ray diffraction with a resolution of 2.90 Å; the secondary structure of this domain is 5% alpha helical and 46% beta sheet (see, e.g., Jarvis et al., J. Med. Chem. 53: 2215-26, 2010). Certain antibodies, binding agents, and small molecules may exhibit binding specificity for one or more extracellular regions of a neuropilin receptor.

Neuropilins can bind to structurally and functionally unrelated ligands (“neuropilin ligands”), such as secreted class III semaphorins, heparin-binding proteins, fibroblast growth factors (e.g., FGF-1, FGF-2, FGF-4, FGF-7), placental growth factors (PGFs), hepatocyte growth factors, and VEGF family members, including, semaphorin 3A, semaphorin 3B, semaphorin 3F, the PLGF-2 isoform of PGF, the VEGF-165 isoform of VEGF (VGF-165), VEGF-A, VEGF-B, and VEGF-C. Semaphorins are proteins initially recognized for their role in neuronal development; they act as axonal chemo-repellants that induce axon growth cone collapse, a process that guides neurons to their ultimate targets in the developing nervous system. VEGFs are signal proteins that stimulates vasculogenesis and angiogenesis, and their overexpression or increased activity can contribute to disease. For example, solid cancers cannot grow beyond a limited size without an adequate blood supply; and cancers that can express VEGF are able to grow and metastasize. Overexpression of VEGF can also cause vascular disease in the retina of the eye and other parts of the body.

Through interactions with these and possibly other ligands, neuropilins play versatile roles in angiogenesis, axon guidance, cell survival, migration, and invasion. In certain instances, increased or aberrant neuropilin expression and/or activity may thus lead to pathologies associated with one or more of aberrant angiogenesis, abnormal axonal growth or development (e.g., increased neurite outgrowth), increased cell survival, increased cell migration, and/or increased cell invasion. Increased activity of one or more neuropilin ligands may also lead the same type of pathologies. GlyRS mutants and related agents may thus be used to reduce the pathology associated with aberrant activation of these and other neuropilin-regulated pathways or processes.

Accordingly, one aspect of the present invention is directed to the use of a mutant GlyRS of the invention, its derivatives, modifications, or small molecules or drugs based on this interaction for the treatment of nervous system injury (Pasterkamp et al., Brain Res. Rev. 35: 36-54, 2001). This includes the treatment of traumatic brain and spinal cord injury including but not limited to neuronal cell apoptosis and scar Connation, neural angiogenesis, and peripheral nerve regeneration. For example, prolonged binding of semaphorin 3A (sem3A) to neuropilin 1 induces cell death (Bagnard et al., J. of Neurosci. 21(10): 3332-41, 2001). Thus, a mutant GlyRS of the invention can act as an antagonist of sem3A-nuropilin-1 interaction to prevent apoptosis and promote cell survival, and possibly proliferation of neuronal stem cells.

The expression of neuropilins is also associated with the pathology of certain cancers see, e.g., Ellis, Mol Cancer Ther. 5:1099-1107, 2006), such as metastatic cancers. Metastasis of cancer cells is a complex process including invasion, hemangiogenesis, lymphangiogenesis, trafficking of cancer cells through blood or lymph vessels, extravasations, organ-specific homing, and growth; it is also a major cause of death. It can be therapeutically desirable to disrupt of one or more of these metastatic processes, for example, to prevent or reduce the likelihood of occurrence of metastasis (before their occurrence), or reduce or reverse the rate of ongoing metastasis. GlyRS mutants and related agents may thus be used to prevent or reduce the pathology associated with metastatic progression of various cancers.

Accordingly, one aspect of the present invention is directed to the use of a mutant GlyRS of the invention, its derivatives, modifications, or small molecules or drugs based on this interaction for the treatment of cancer because neuropilin, including but not limited to metastatic prostate tumor cells, breast carcinomas, solid tumor growth, leukemia, metastasis and melanoma (Soker et al., Cell 92:735-745, 1998; and Latil et al., Int. J Cancer 89: 167-171, 2000). Neuropilin expression by tumor cells promotes tumor angiogenesis and progression (Miao et al., FASEB J. 14: 2532-2539, 2000). Furthermore, increased expression of endothelial neuropilin is associates with neuroblastoma stages I-IV (Fakhari et al., Cancer 94(1): 258-263, 2001).

Another aspect of the present invention is directed to the use of a mutant GlyRS of the invention, its derivatives, modifications, or small molecules or drugs based on this interaction for the treatment of rheumatoid arthritis. Neuropilin up-regulation has been detected in the synovial tissues of rheumatoid arthritis patients and this correlated with increased vascular density (Ikeda et al., J. of Path. 191: 426-433, 2000).

Another aspect of the present invention is directed to the use of a mutant GlyRS of the invention, its derivatives, modifications, or small molecules or drugs based on this interaction for the treatment of organogenesis and development anomalies. Both semaphorin Ill (nerves, bones, and heart) (Behar et al., Nature 383: 525-528, 1996) and neuropilin-1 (blood vessel) (Kawasaki et al., Development 126: 4895-4902, 1999) have been shown to be critical cues for appropriate embryonic development. Furthermore, a double knockout of NRP-1 and NRP-2 revealed more severe vascular defects, suggesting that both are required for appropriate blood vessel development (Takashima et al., Proc. Natl. Acad. Sci. 99: 3657-3662, 2002).

Another aspect of the present invention is directed to the use of a mutant GlyRS of the invention, its derivatives, modifications, or small molecules or drugs based on this interaction for the treatment of ocular-related diseases such as Proliferate Diabetic Retinopathy, retinal neovascularization, neovascular glaucoma, diabetic retinopathy and Age Related Macular Degeneration, because neuropilin-1 and KDR/Flk-1 have been found to co-localized in the area of neovascularized vessels of the retina (Maramatsu et al., Inves Ophthalmol Vis. Sci. 42(6): 1172-8, 2001; and Ikeda et al., Inves Opthalmol Vis. Sci. 41(7): 1649-56, 2000).

Another aspect of the present invention is directed to the use a mutant GlyRS of the invention, its derivatives, modifications, or small molecules or drugs based on this interaction for the treatment of angiogenesis diseases associated with inappropriate arterial-venous junctions or conversion such as in vein graft stenosis, because neuropilin-1 is preferentially express by arterial and neuropilin-2 is preferentially expressed by venous blood vessels (Herzog et al., Mech. Dev. 109 (1): 115-9, 2001).

Certain embodiments include methods of reducing or blocking neuropilin activity or activation in a neuropilin-expressing cell, including, for example, a cell that over-expresses membrane bound or soluble neuropilin or that is subject to increased activation by one or more neuropilin ligands, comprising contacting the neuropilin or neuropilin-expressing cell with a human glycyl-tRNA synthetase (GlyRS) mutant or related agent described herein, thereby reducing neuropilin activity in the cell or surrounding environment. Typically, the GlyRS mutant or related agent comprises one or more exposed neomorphic regions of GlyRS, as described herein, such that its affinity for neuropilin is greater than the affinity of wild-type human GlyRS for neuropilin by at least about 1.5× to at least about 100× or to at least about 1000×, and is thus capable of competitively inhibiting the binding of neuropilin to one or more of its ligands. In some aspects, the mutant GlyRS will exhibit an apparent affinity for neuropilin-1 in a BIACORE assay of at least about 10 nM, at least about 20 nM, at least about 50 nM, at least about 100 nM, at least about 200 nM, or at least about 500 nM. General examples of ligands include vascular endothelial growth factor (VEGF), semaphorins, heparin-binding proteins, fibroblast growth factor-2 (FGF-2), placental growth factors (PGFs), and hepatocyte growth factor (HOF). Specific examples of ligands include VEGF-165, VEGF-B, and sema3A, among others described herein and known in the art. The methods may therefore be reduce activation of neuropilin that is associated with aberrant or increased activity, expression, or levels of one or more of VEGFs, FGF-2, heparin-binding proteins, PGFs, HGF, and/or semaphorins.

Generally, as noted above, the (typically aberrant) neuropilin activity can be associated with one or more of angiogenesis, axonal growth or development, cell migration, cell invasion, cell metastasis, and/or cell adhesion. The neuropilin-expressing cell can be in a subject, optionally where the subject has a disease or condition associated with increased neuropilin activity, and/or increased activity of one or more neuropilin ligands. For instance, the neuropilin ligand sema3A has been associated with inhibition of re-myelination, and increased sema3A activity may thus negatively impact myelin regeneration in diseases such as multiple sclerosis (MS) (see, e.g., Syed et al., The Journal of Neuroscience. 31:3719-3728, 2011). Polypeptides or other agents comprising one or more exposed GlyRS neomorphic regions may thus be used to treat these and other neuropilin or neuropilin-ligand-related conditions.

In some embodiments, the cell is a cancer cell, and the subject has cancer, including cancer that is not yet metastatic, but is at some risk for becoming metastatic. In specific embodiments, the cell is a metastatic cancer cell, and the subject has at least one cancer cell that has spread from its original tissue to one or more other tissues. In certain embodiments, the subject has been diagnosed more generally as having a type of cancer that associates with increased neuropilin expression or activity, and/or has been diagnosed specifically as having increased neuropilin expression or activity (see, e.g., Ellis, Mol Cancer Ther. 5:1099-1107, 2006; and Yasuoka et al., BMC Cancer. 9:220, 2009). Examples of cancers include prostate cancer, breast cancer, colon cancer, rectal cancer, lung cancer, astrocytoma, ovarian cancer, testicular cancer, stomach cancer, bladder cancer, pancreatic cancer, liver cancer, kidney cancer, brain cancer, melanoma, non-melanoma skin cancer, bone cancer, lymphoma, leukemia, thyroid cancer, endometrial cancer, multiple myeloma, acute myeloid leukemia, neuroblastoma, glioblastoma, or non-Hodgkin's lymphoma, among others known in the art. Accordingly, GlyRS mutant polypeptides comprising one or more exposed neomorphic regions, or other polypeptides/agents having such exposed regions, may be used to reduce growth or metastasis of any one or more of these and other cancers.

In one aspect of any of these methods the GlyRS mutant polypeptides comprise at least one disease associated mutation. In some embodiments, such disease-associated mutant GlyRS polypeptides differ from SEQ ID NO: 1 by at least one amino acid selected from the group consisting of A57V, E71G, L129P, C157R, P234KY, G240R, P244L, I280F, H418R, D500N, G526R, S581L, and G598A.

Upon diagnosis, an appropriate treatment regime can be established according to routine knowledge in the art. Generally, a therapeutically effective amount of a compound (e.g., antibody, binding agent, small molecule, mutant GlyRS, neuropilin) is administered to a subject or patient. In particular embodiments, the amount of compound administered will typically be in the range of about 0.1 μg/kg to about 0.1 mg/kg to about 50 mg/kg of patient body weight. Depending on the type and severity of the disease, about 0.1 μg/kg to about 0.1 mg/kg to about 50 mg/kg body weight (e.g., about 0.1-15 mg/kg/dose) of compound can be an initial candidate dosage for administration to the patient, whether, for example, by one or more separate administrations, or by continuous infusion. For example, a dosing regimen may comprise administering an initial loading dose of about 4 mg/kg, followed by a weekly maintenance dose of about 2 mg/kg of the compound, or about half of the loading dose. However, other dosage regimens may be useful. A typical daily dosage might range from about 1 μg/kg to 100 mg/kg or more, depending on the factors mentioned above.

Other exemplary dosage regimes are described elsewhere herein. For repeated administrations over several days or longer, depending on the condition, the treatment can be sustained until a desired suppression of disease symptoms occurs. The progress of these and other therapies can be readily monitored by conventional methods and assays and based on criteria known to the physician or other persons of skill in the art.

### Drug Discovery

Certain embodiments relate to the use of the neuronal disease-associated (e.g., Charcot-Marie-Tooth (CMT) disease-associated) GlyRS neomorphic regions described herein for drug discovery, for instance, to identify agents that modulate one or more of the disease-associated, non-canonical activities of a GlyRS mutant of interest. For example, certain embodiments include methods of identifying one or more “cellular binding partners” of a disease-associated GlyRS mutant, such as a membrane bound or extracellular protein or other host molecule that directly or physically interacts with said GlyRS mutant. Particular examples of cellular binding partners include secreted proteins, cell-surface receptors and extracellular domains thereof, which preferably participate in downstream activities related to pathology of a neuronal disease such as a CMT disease.

Also included are methods of identifying host molecules that participate in one or more disease-associated activities of a GlyRS mutant, including molecules that directly or indirectly interact with the cellular binding partner, and either regulate its role in a non-canonical activity, or are regulated by the binding partner. Such host molecules include both upstream and downstream components of the non-canonical, neuronal disease-associated pathway, typically related by about 1, 2, 3, 4, 5 or more identifiable steps in the pathway, relative to the cellular binding partner/GlyRS mutant interaction. Such cellular binding partners include the neuropilin transmembrane receptors, such as neuropilin-1 and neuropilin-2, and their associated ligands, as described herein and known in the art.

Certain aspects include methods of identifying a compound (e.g., antibody, binding agent, small molecule) or other agent that agonizes the disease-associated non-canonical activity of a GlyRS mutant, such as by interacting with GlyRS mutant and/or one or more of its cellular binding partners, such as a neuropilin transmembrane receptor. Examples of disease-associated GlyRS mutants are described elsewhere herein and known in the art, and include, for example, A57V, E71G, L129P, C157R, P234KY, G240R, P244L, I280F, H418R, D500N, G526R, S581L, and G598A mutants, in addition to GlyRS proteins having a full or partial deletions of the WHEP region.

Certain embodiments therefore include methods of identifying a cellular binding partner of disease-associated GlyRS mutant, comprising a) combining a polypeptide that comprises an exposed neomorphic region of human GlyRS, for example, where said neomorphic region is one or more of A57-A663, A57-A83, L129-D161, N208-Y320, V366-H378, P518-M531, L584-Y604, F620-R635, or D654-A663, or an antigenic fragment or combination of said region(s), with a biological sample under suitable conditions, and b) detecting specific binding of the neomorphic region to a binding partner, thereby identifying a binding partner that specifically binds to the neomorphic region of the GlyRS mutant. Also included are methods of screening for a compound that specifically binds to a neomorphic region of human GlyRS, comprising a) combining a polypeptide that comprises an exposed neomorphic region of human GlyRS, for example, where said neomorphic region is one or more of A57-A663, A57-A83, L129-D161, N208-Y320, V366-H378, P518-M531, L584-Y604, F620-R635, or D654-A663, or an antigenic fragment or combination of said region(s), with at least one test compound under suitable conditions, optionally in the presence of a cellular binding partner such as a neuropilin (NRP1, NRP2, etc.), and b) detecting binding of the neomorphic region of the polypeptide to the test compound, thereby identifying a compound that specifically binds to a neomorphic region of human GlyRS, preferably an exposed neomorphic region of human GlyRS. When the cellular binding partner is present, the methods can also include detecting the ability of test compound to reduce binding between the disease-associated GlyRS mutant (or other polypeptide comprising an exposed neomorphic region of GlyRS, as described herein) and the cellular binding partner. In specific embodiments, the cellular binding partner is a neuropilin, and the test compound fully or partially antagonizes the interaction between the disease-associated GlyRS mutant and the neuropilin.

In certain embodiments, the GlyRS mutant of these and related methods is properly folded, relative, for example, to its native or most stable three-dimensional conformation in a cell or a physiological solution, and one or more of the neomorphic regions (or hot spots) are found on an exposed surface of the protein. Certain embodiments may employ a properly folded wild-type GlyRS (relative, for example, to its native or most stable conformation in a cell or a physiological solution) as a negative control. In certain embodiments, the compound is an antibody, polypeptide, or peptide. In certain embodiments, the compound is a small molecule or other (e.g., non-biological) chemical compound. In certain embodiments, the compound is a peptide mimetic.

Any method suitable for detecting protein-protein interactions may be employed for identifying cellular proteins that interact with a GlyRS neomorphic region, interact with one or more of its cellular binding partners, or both. Examples of traditional methods that may be employed include co-immunoprecipitation, cross-linking, and co-purification through gradients or chromatographic columns of cell lysates or proteins obtained from cell lysates, mainly to identify proteins in the lysate that interact with the neomorphic region.

In these and related embodiments, at least a portion of the amino acid sequence of a protein that interacts with a GlyRS neomorphic region or its binding partner can be ascertained using techniques well known to those of skill in the art, such as via the Edman degradation technique. See, e.g., Creighton Proteins: Structures and Molecular Principles, W. H. Freeman & Co., N.Y., pp. 34 49, 1983. The amino acid sequence obtained may be used as a guide for the generation of oligonucleotide mixtures that can be used to screen for gene sequences encoding such proteins. Screening may be accomplished, for example, by standard hybridization or PCR techniques, as described herein and known in the art. Techniques for the generation of oligonucleotide mixtures and the screening are well known. See, e.g., Ausubel et al. Current Protocols in Molecular Biology Green Publishing Associates and Wiley Interscience, N. Y., 1989; and Innis et al., eds. PCR Protocols: A Guide to Methods and Applications Academic Press, Inc., New York, 1990.

Additionally, methods may be employed in the simultaneous identification of genes that encode the binding partner or other polypeptide. These methods include, for example, probing expression libraries, in a manner similar to the well known technique of antibody probing of lambda-gt11 libraries, using labeled GlyRS neomorphic region-containing proteins, or another polypeptide, peptide or fusion protein, e.g., a variant neomorphic region polypeptide or GlyRS neomorphic region fused to a marker (e.g., an enzyme, fluor, luminescent protein, or dye) or an Ig-Fc domain.

One method that detects protein interactions in vivo, the two-hybrid system, is described in detail for illustration only and not by way of limitation. One example of this system has been described (Chien et al., PNAS USA 88:9578 9582, 1991) and is commercially available from Clontech (Palo Alto, Calif.).

Briefly, utilizing such a system, plasmids may be constructed that encode two hybrid proteins: one plasmid consists of nucleotides encoding the DNA-binding domain of a transcription activator protein fused to one or more GlyRS neomorphic region-encoding nucleotide sequence(s), (or, in certain embodiments, its binding partner), preferably in context of a protein that allows the neomorphic region to be exposed on the protein surface, and the other plasmid consists of nucleotides encoding the transcription activator protein's activation domain fused to a cDNA (or collection of cDNAs) encoding an unknown protein(s) that has been recombined into the plasmid as part of a cDNA library. The DNA-binding domain fusion plasmid and the activator cDNA library may be transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or other such methodology may be used to screen activation domain libraries for proteins that interact with the “bait” gene product. By way of example, and not by way of limitation, a GlyRS neomorphic region-containing protein or neomorphic region peptide may be used as the bait gene product. A cellular binding partner may also be used as a “bait” gene product. Total genomic or cDNA sequences are fused to the DNA encoding an activation domain. This library and a plasmid encoding a hybrid of a bait GlyRS-containing gene product fused to the DNA-binding domain are co-transformed into a yeast reporter strain, and the resulting transformants are screened for those that express the reporter gene.

A cDNA library of the cell line from which proteins that interact with bait GlyRS neomorphic regions are to be detected can be made using methods routinely practiced in the art. For example, the cDNA fragments can be inserted into a vector such that they are translationally fused to the transcriptional activation domain of GAL4. This library can be co-transformed along with the bait gene-GAL4 fusion plasmid into a yeast strain, which contains a lacZ gene driven by a promoter that contains GAL4 activation sequence. A cDNA encoded protein, fused to GAL4 transcriptional activation domain, that interacts with bait gene product will reconstitute an active GAL4 protein and thereby drive expression of the HIS3 gene. Colonies, which express HIS3, can be detected by their growth on Petri dishes containing semi-solid agar based media lacking histidine. The cDNA can then be purified from these strains, and used to produce and isolate the bait GlyRS neomorphic region gene-interacting protein using techniques routinely practiced in the art.

Also included are three-hybrid systems, which allow the detection of RNA-protein interactions in yeast. See, e.g., Hook et al., RNA. 11:227-233, 2005. Accordingly, these and related methods can be used to identify a cellular binding partner of a GlyRS neomorphic region, and to identify other proteins or nucleic acids that interact with the region(s), the cellular binding partner, or both.

Certain embodiments relate to the use of interactome screening approaches. Particular examples include protein domain-based screening (see, e.g., Boxem et al., Cell. 134:534-545, 2008; and Yu et al., Science. 322:10-110, 2008).

As noted above, once isolated, binding partners can be identified and can, in turn, be used in conjunction with standard techniques to identify proteins or other compounds with which it interacts. Certain embodiments thus relate to methods of screening for a compound that specifically binds to the binding partner of a GlyRS neomorphic region-containing polypeptide or peptide, comprising a) combining the binding partner with at least one test compound under suitable conditions, and b) detecting binding of the binding partner to the test compound, thereby identifying a compound that specifically binds to the binding partner. In certain embodiments, the test compound is an antibody, polypeptide, or other binding agent. In certain embodiments, the test compound is a chemical compound, such as a small molecule compound or peptide mimetic. In specific embodiments, the binding partner is a neuropilin transmembrane receptor, such as neuropilin-1 or neuropilin-2, or a polypeptide that comprises all or a portion of the extracellular domain of a neuropilin.

Certain embodiments include methods of screening for a compound that modulates the activity of a disease-associated GlyRS mutant, comprising a) combining a polypeptide that comprises or consists of at least one neomorphic region of human GlyRS, for example, where said neomorphic region is one or more of A57-A663, A57-A83, L129-D161, N208-Y320, V366-H378, P518-M531, L584-Y604, F620-R635, or D654-A663, or a fragment or combination of said region(s), with at least one test compound under conditions permissive for the activity of the polypeptide, for example, in the optional presence of an isolated cellular binding partner or a cell that expresses a cellular binding partner, such as a neuropilin, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide. Certain embodiments include methods of screening for a compound that modulates the activity of a binding partner of a disease-associated GlyRS mutant, comprising a) combining the binding partner with at least one test compound under conditions permissive for the activity of the binding partner, b) assessing the activity of the binding partner in the presence of the test compound, and c) comparing the activity of the binding partner in the presence of the test compound with the activity of the binding partner in the absence of the test compound, wherein a change in the activity of the binding partner in the presence of the test compound is indicative of a compound that modulates the activity of the binding partner. Typically, these and related embodiments include assessing a selected non-canonical activity that is associated with a neuronal disease, as described herein. Included are in vitro and in vivo conditions, such as cell culture conditions.

One specific assay includes measuring the ability of a test compound to reduce the ability of disease-associated GlyRS mutants to inhibit neuropilin-mediated neurite outgrowth. As shown in the accompanying Examples (see Example 6), for example, certain GlyRS mutants but not wild-type GlyRS inhibit neuropilin-induced neurite outgrowth in N2a cells, and test compounds could be added to this system (or a similar system) to measure their ability to reverse this process, and restore neurite outgrowth, relative to a control test compound or no test compound.

Also included are methods of screening a compound for effectiveness as a full or partial antagonist of a disease-associated GlyRS mutant, comprising a) exposing a sample comprising a test polypeptide to a compound, where the test polypeptide comprises or consists of at least one neomorphic region of human GlyRS, for example, where said neomorphic region is one or more of A57-A663, A57-A83, L129-D161, N208-Y320, V366-H378, P518-M531, L584-Y604, F620-R635, or D654-A663, or a fragment or combination of said region(s), optionally in the presence of a cellular binding partner of the test polypeptide or a cell that expresses such as cellular binding partner, and b) detecting antagonist activity in the sample, typically by measuring or determining a decrease in the non-canonical activity the test polypeptide, preferably determining a decrease in neuronal disease-associated or CMT-associated activity. One example of a cellular binding partner is a neuropilin transmembrane receptor. Certain methods include a) exposing a sample comprising a binding partner of the disease-associated GlyRS mutant to a compound, and b) detecting antagonist activity in the sample, typically by measuring a decrease in the selected non-canonical activity of the GlyRS mutant. One example of a non-canonical activity is the inhibition of neuropilin-mediated neurite outgrowth, or the inhibition of another neuropilin-mediated cellular phenotype. Certain embodiments include compositions that comprise an antagonist compound identified by the method and a pharmaceutically acceptable carrier or excipient.

Certain embodiments include methods for identifying a compound that modulates a disease mediated by a human mutant tRNA synthetase, comprising a) incubating a test compound with the mutant tRNA synthetase, and b) measuring the rate of deuterium exchange in the presence of the test compound relative to the rate of deuterium exchange in the absence of the test compound, where a difference in the rates of deuterium exchange in the presence and absence of the test compound indicates that the compound is capable of modulating a disease mediated by the human mutant tRNA synthetase. In specific embodiments, the mutant tRNA synthetase is a mutant glycyl-tRNA synthetase associated with a neuronal disease, such as CMT. In some embodiments, the mutant tRNA synthetase is disease-associated tyrosyl-tRNA synthetase (TyrRS), alanyl-tRNA synthetase (AlaRS), or lysyl-tRNA synthetase (LysRS). In some embodiments, the compound is an antibody or a binding agent, or a small molecule.

Also included are processes for manufacturing a pharmaceutical composition, wherein said composition comprises an antibody, binding agent or small molecule, comprising: a) performing an in vitro screen of one or more test antibodies, binding agents or small molecules in the presence of a polypeptide that comprises an exposed neomorphic region of human GlyRS, where said neomorphic region is one or more of A57-A663, A57-A83, L129-D161, N208-Y320, V366-H378, P518-M531, L584-Y604, F620-R635, or D654-A663, or a combination or an antigenic fragment of said region(s), to identify an antibody, binding agent or small molecule that specifically binds to the neomorphic region; b) performing a cell-based assay with the antibody, binding agent or small molecule identified in step a), to identify an antibody, binding agent or small molecule that modulates one or more non-canonical activities of a human GlyRS mutant associated with Charcot-Marie-Tooth Disease Type 2D or Distal Spinal Muscular Atrophy Type V (dSMA-V); c) optionally assessing the structure-activity relationship (SAR) of the antibody, binding agent or small molecule identified in step b), to correlate its structure with modulation of the non-canonical activity, and optionally derivatizing the antibody, binding agent or small molecule to alter its ability to modulate the non-canonical activity; and d) producing sufficient amounts of the antibody, binding agent or small molecule identified in step b), or the derivatized antibody or binding agent in step c), for use in humans, thereby manufacturing the pharmaceutical composition. In certain embodiments, the polypeptide is a fusion protein that comprises at least one heterologous sequence and one or more of said neomorphic regions. In some embodiments, polypeptide consists essentially of A57-A663, A57-A83, L129-13161, N208-Y320, V366-H378, P518-M531, L584-Y604, F620-R635, or D654-A663 of human GlyRS, or combination or an antigenic fragment thereof. In certain embodiments, the binding agent is selected from the group consisting of adnectins, anticalins, avimers, DARPins, and aptamers. In specific embodiments, the antibody or binding agent fully or partially antagonizes an interaction between a human GlyRS mutant associated with a Charcot-Marie-Tooth Disease Type 2D and its cellular binding partner(s).

Also included are methods of identifying an antibody, binding agent or small molecule that, reduces binding between a glycyl-tRNA synthetase (GlyRS) mutant and a neuropilin transmembrane receptor, such as neuropilin-1 or neuropilin-2, wherein the GlyRS mutant comprises one or more exposed neomorphic regions, comprising a) combining a first polypeptide that comprises an exposed neomorphic region of human GlyRS, where said neomorphic region is one or more of A57-A663, A57-A83, L129-D161, N208-Y320, V366-H378, P518-M531, L584-Y604, F620-R635, or D654-A663, or a combination or an antigenic fragment of said region(s), with at least one test antibody, binding agent or small molecule, and a neuropilin polypeptide under suitable conditions, for example, where the neuropilin is expressed in the surface of a cell, is part of an in vitro solution, or is anchored to a solid substrate, and b) detecting or determining ability of the test antibody, binding agent or small molecule to reduce binding between the first polypeptide and the neuropilin polypeptide, relative to a control sample without the test antibody, binding agent or small molecule, where reduced binding is statistically significant, thereby identifying an antibody, binding agent or small molecule that reduces binding between a GlyRS mutant and neuropilin transmembrane receptor.

In certain embodiments, in vitro systems may be designed to identify compounds capable of interacting with or modulating a disease-associated GlyRS neomorphic sequence or its binding partner. Certain of the compounds identified by such systems may be useful, for example, in modulating the activity of the pathway, and in elaborating components of the pathway itself. They may also be used in screens for identifying compounds that disrupt interactions between components of the pathway; or may disrupt such interactions directly. One exemplary approach involves preparing a reaction mixture of a polypeptide comprising or consisting of a GlyRS neomorphic region and a test compound under conditions and for a time sufficient to allow the two to interact and bind, thus forming a complex that can be removed from and/or detected in the reaction mixture.

In vitro screening assays can be conducted in a variety of ways. For example, a polypeptide comprising or consisting of a GlyRS neomorphic region, or a cellular binding partner thereof such as a neuropilin, and/or one or more test compound(s) can be anchored onto a solid phase. In these and related embodiments, the resulting complexes may be captured and detected on the solid phase at the end of the reaction. In one example of such a method, the GlyRS neomorphic sequence and/or its binding partner are anchored onto a solid surface, and the test compound(s), which are not anchored, may be labeled, either directly or indirectly, so that their capture by the component on the solid surface can be detected. In other examples, the test compound(s) are anchored to the solid surface, and the GlyRS neomorphic sequence and/or its binding partner such as a neuropilin, which are not anchored, are labeled or in some way detectable. In certain embodiments, microtiter plates may conveniently be utilized as the solid phase. The anchored component (or test compound) may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored, if desired.

To conduct an exemplary assay, the non-immobilized component is typically added to the coated surface containing the anchored component. After the reaction is complete, un-reacted components are removed (e.g., by washing) under conditions such that any specific complexes formed will remain immobilized on the solid surface. The detection of complexes anchored on the solid surface can be accomplished in a number of ways. For instance, where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, the presence or absence of binding of a test compound can be determined, for example, using surface plasmon resonance (SPR) and the change in the resonance angle as an index, wherein a polypeptide comprising or consisting of a GlyRS neomorphic region or a cellular binding partner such as a neuropilin (or a polypeptide or other molecule that comprises all or a portion of a neuropilin extracellular domain) is immobilized onto the surface of a commercially available sensorchip (e.g., manufactured by BIACORE™) according to a conventional method, the test compound is contacted therewith, and the sensorchip is illuminated with a light of a particular wavelength from a particular angle. The binding of a test compound can also be measured by detecting the appearance of a peak corresponding to the test compound by a method where a GlyRS neomorphic region or a cellular binding partner is immobilized onto the surface of a protein chip adaptable to a mass spectrometer, a test compound is contacted therewith, and an ionization method such as MALDI-MS, ESI-MS, FAB-MS and the like is combined with a mass spectrometer (e.g., double-focusing mass spectrometer, quadrupole mass spectrometer, time-of-flight mass spectrometer, Fourier transformation mass spectrometer, ion cyclotron mass spectrometer and the like).

In certain embodiments, cell-based assays, membrane vesicle-based assays, or membrane fraction-based assays can be used to identify compounds that modulate interactions in the non-canonical pathway of a selected disease-associated GlyRS mutant. To this end, cell lines that express a GlyRS neomorphic region-containing polypeptide and/or a binding partner such as a neuropilin (e.g., neuropilin-1, neuropilin-2), or a fusion protein containing a domain or fragment of such proteins (or a combination thereof), or cell lines (e.g., COS cells, CHO cells, HEK293 cells, Hela cells etc.) that have been genetically engineered to express such protein(s) or fusion protein(s) can be used. Test compound(s) that influence the non-canonical activity can be identified by monitoring a change (e.g., a statistically significant change) in that activity as compared to a control or a predetermined amount. One exemplary non-canonical activity is the inhibition of neuropilin-mediated neurite outgrowth, for example, where the test compound restores neuropilin-mediated neurite outgrowth, relative to a control compound or no test compound. However, these and related methods can be applied to any neuropilin-mediated activity that is otherwise inhibited by the presence of the disease-associated GlyRS mutant.

Also included are in vivo assays, e.g., animal models, for identifying or optimizing compounds that reduce or ameliorate one or more symptoms of a GlyRS-associated disease, such as CMT. Examples of in vivo animal models include the mouse model of CMT2D, having an active dominant mutation of GlyRS, as described, for example, in Sebum et al., Neuron. 51:715-726, 2006.

Antibodies to GlyRS neomorphic regions can also be used in screening assays, such as to identify an agent that specifically binds to the neomorphic region, confirm the specificity or affinity of an agent that binds to the neomorphic region, or identify the specific site of interaction between the agent and the neomorphic region. Included are assays in which the antibody is used as a competitive inhibitor of the agent. For instance, an antibody that specifically binds to a GlyRS neomorphic region with a known affinity can act as a competitive inhibitor of a selected agent, and be used to calculate the affinity of the agent for the neomorphic region. Also, one or more antibodies that specifically bind to known epitopes or sites within a neomorphic region can be used as a competitive inhibitor to confirm whether or not the agent binds at that same site. Other variations will be apparent to persons skilled in the art.

Also included are any of the above methods, or other screening methods known in the art, which are adapted for high-throughput screening (HTS). HTS typically uses automation to run a screen of an assay against a library of candidate compounds, for instance, an assay that measures an increase or a decrease in a non-canonical activity, as described herein.

Any of the screening methods provided herein may utilize small molecule libraries or libraries generated by combinatorial chemistry. Libraries of chemical and/or biological mixtures, such as fungal, bacterial, or algal extracts, are known in the art and can be screened with any of the assays of the invention. Examples of methods for the synthesis of molecular libraries can be found in: (Carell et al., 1994a; Carell et al., 1994b; Cho et al., 1993; DeWitt et al., 1993; Gallop et al., 1994; Zuckermann et al., 1994).

Libraries of compounds may be presented in solution (Houghten et al., 1992) or on beads (Lam et al., 1991), on chips (Fodor et al., 1993), bacteria, spores (Ladner et al., U.S. Pat. No. 5,223,409, 1993), plasmids (Cull et al., 1992) or on phage (Cwirla et al., 1990; Devlin et al., 1990; Felici et al., 1991; Ladner et al., U.S. Pat. No. 5,223,409, 1993; Scott and Smith, 1990). Embodiments of the present invention encompass the use of different libraries for the identification of small molecule modulators of one or more CMT-associated GlyRS mutants, their cellular binding partners, and/or their related non-canonical activities. Libraries useful for the purposes of the invention include, but are not limited to, (1) chemical libraries, (2) natural product libraries, and (3) combinatorial libraries comprised of random peptides, oligonucleotides and/or organic molecules.

Chemical libraries consist of structural analogs of known compounds or compounds that are identified as “hits” or “leads” via natural product screening. Natural product libraries are derived from collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. See, e.g., Cane et al., Science 282:63-68, 1998. Combinatorial libraries may be composed of large numbers of peptides, oligonucleotides or organic compounds as a mixture. They are relatively easy to prepare by traditional automated synthesis methods, PCR, cloning or proprietary synthetic methods.

More specifically, a combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

For a review of combinatorial chemistry and libraries created therefrom, see, e.g., Huc, I. and Nguyen, R. (2001) Comb. Chem. High Throughput Screen 4:53-74; Lepre, C A. (2001) Drug Discov. Today 6:133-140; Peng, S. X. (2000) Biomed. Chromatogr. 14:430-441; Bohm, H. J. and Stahl, M. (2000) Curr. Opin. Chem. Biol. 4:283-286; Barnes, C and Balasubramanian, S. (2000) Curr. Opin. Chem. Biol. 4:346-350; Lepre, Enjalbal, C, et al., (2000) Mass Septrom Rev. 19:139-161; Hall, D. G., (2000) Nat. Biotechnol. 18:262-262; Lazo, J. S., and Wipf, P. (2000) J. Pharmacol. Exp. Ther. 293:705-709; Houghten, R. A., (2000) Ann. Rev. Pharmacol. Toxicol. 40:273-282; Kobayashi, S. (2000) Curr. Opin. Chem. Biol. (2000) 4:338-345; Kopylov, A. M. and Spiridonova, V. A. (2000) Mol. Biol. (Mosk) 34:1097-1113; Weber, L. (2000) Curr. Opin. Chem. Biol. 4:295-302; Dolle, R. E. (2000) J. Comb. Chem. 2:383-433; Floyd, C D., et al., (1999) Prog. Med. Chem. 36:91-168; Kundu, B., et al., (1999) Prog. Drug Res. 53:89-156; Cabilly, S. (1999) Mol. Biotechnol. 12:143-148; Lowe, G. (1999) Nat. Prod. Rep. 16:641-651; Dolle, R. E. and Nelson, K. H. (1999) J. Comb. Chem. 1:235-282; Czarnick, A. W. and Keene, J. D. (1998) Curr. Biol. 8:R705-R707; Dolle, R. E. (1998) Mol. Divers. 4:233-256; Myers, P. L., (1997) Curr. Opin. Biotechnol. 8:701-707; and Pluckthun, A. and Cortese, R. (1997) Biol. Chem. 378:443.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Biosciences, Columbia, Md.).

### Diagnostic Methods

Antibodies, binding agents, and small molecules described herein that exhibit binding specificity for a disease-associated glycyl-tRNA synthetase (GlyRS) can be used in diagnostic assays and diagnostic compositions. Included are biochemical, histological, and cell-based methods and compositions, among others.

Accordingly, certain embodiments include methods of diagnosing a neuronal disease such as Charcot-Marie-Tooth (CMT) disease in a subject, comprising contacting a biological sample from said subject with an antibody or antigen-binding fragment described herein, a binding agent, or small molecule described herein, where determining or detecting a specific interaction between said antibody or antigen-binding fragment, or said binding agent or small molecule, and a GlyRS in said sample indicates that the subject has a neuronal disease, such as CMT disease.

Certain aspects can employ the antibodies, binding agents, and other compounds described herein as part of a companion diagnostic method, to assess whether a subject or population subjects will respond favorably to a specific medical treatment. For instance, a given therapeutic agent (e.g., antibody, binding agent, small molecule) could be identified as suitable for a subject or certain populations of subjects based on whether the subject(s) have one or more selected biomarkers or mutations for a given disease or condition. Examples of disease-associated GlyRS mutations are described herein.

Because different mutations may result in exposure of different neomorphic regions on the surface of GlyRS, the identification of a specific mutation in a subject could indicate which specific antibody or binding agent is best suited for treating that subject. For example, subjects diagnosed with a L129P mutation may show greater exposure of neomorphic regions A57-A83, G97-T110, E119-5178, N208-Y320, A326-N348, L361-H378, K423-E429, V461-Y464, L480-F486, K505-P554, V564-N570, L584-Y604, F620-I645, and/or D654-A663, and may be optimally treated with antibodies, binding agents and/or small molecules to one or more of these regions. As another example, subjects diagnosed with G240R may show greater exposure of neomorphic regions A57-A83, G97-E123, F147-L189, F204-Y320, N348-H378, V461-Y464, K483-M531, D545-R642, and/or D654-E685, and may be optimally treated with antibodies, binding agents and/or small molecules to one or more of these regions. As another example, subjects diagnosed with a G526R mutant may show greater exposure of neomorphic regions A57-A83, L129-K150, S183-V188, N208-Y320, N348-D389, K423-E429, L480-E485, D500-L511, P518-M531, T538-F550, L584-Y604, F620-I645, and/or D654-A663, and can be optionally treated with antibodies, binding agents and/or small molecules to one or more of these regions. Subjects diagnosed with a S581L mutant may show greater exposure of neomorphic regions A57-107, L129-D161, N208-Y320, V366-I402, K493-Q496, V513-M531, A555-R635, and/or D654-E685, and may be optionally treated with antibodies, binding agents and/or small molecules to one or more of these regions. Subjects diagnosed with a G598A mutant may show greater exposure of neomorphic regions A57-N106, L129-L203, N208-Y320, V366-D389, A421-Y464, E504-M531, F551-I645, and/or D654-A663, and may be optionally treated with antibodies, binding agents and/or small molecules to one or more of these regions.

Embodiments of the present invention therefore include a variety of polypeptide-based detection techniques, including antibody-based detection techniques. Included in these embodiments are the use of GlyRS polypeptides (comprising one or more neomorphic regions) to generate antibodies, binding agents and/or small molecules, which may then be used in diagnostic methods and compositions to detect or quantitate disease-associated GlyRS mutations in a cell or other biological sample, typically from a subject.

Certain embodiments may employ standard methodologies and detectors such as western blotting and immunoprecipitation, enzyme-linked immunosorbent assays (ELISA), flow cytometry, and immunofluorescence assays (IFA), which utilize an imaging device. These well-known methods typically utilize one or more monoclonal or polyclonal antibodies as described herein that specifically bind to a selected GlyRS mutant, or a unique region of that GlyRS mutant, and generally do not bind significantly to wild-type GlyRS, preferably in its native three-dimensional conformation. In certain embodiments, the unique region of the GlyRS mutant may represent an entirely unique three-dimensional structure, or uniquely exposed three-dimensional structure.

Certain embodiments may employ “arrays,” such as “microarrays.” In certain embodiments, a “microarray” may also refer to a “peptide microarray” or “protein microarray” having a substrate-bound collection or plurality of polypeptides, the binding to each of the plurality of bound polypeptides being separately detectable. Alternatively, the peptide microarray may have a plurality of binders, including but not limited to monoclonal antibodies, polyclonal antibodies, phage display binders, yeast 2 hybrid binders, and aptamers, which can specifically detect the binding of the disease-associated GlyRS mutant. The array may be based on autoantibody detection of GlyRS mutants, as described, for example, in Robinson et al., Nature Medicine 8(3):295-301 (2002). Examples of peptide arrays may be found in WO 02/31463, WO 02/25288, WO 01/94946, WO 01/88162, WO 01/68671, WO 01/57259, WO 00/61806, WO 00/54046, WO 00/47774, WO 99/40434, WO 99/39210, and WO 97/42507 and U.S. Pat. Nos. 6,268,210, 5,766,960, and 5,143,854, each of which are incorporated by reference.

Certain embodiments may employ cell-sorting or cell visualization or imaging devices/techniques to detect or quantitate the presence or levels of a GlyRS mutant. Examples include flow cytometry or FACS, immunofluorescence analysis (IFA), and in situ hybridization techniques, such as fluorescent in situ hybridization (FISH).

Certain embodiments may employ conventional biology methods, software and systems for diagnostic purposes. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001): See U.S. Pat. No. 6,420,108.

Certain embodiments may employ various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

The whole genome sampling assay (WGSA) is described, for example in Kennedy et al., Nat. Biotech. 21, 1233-1237 (2003), Matsuzaki et al., Gen. Res. 14: 414-425, (2004), and Matsuzaki, et al., Nature Methods 1:109-111 (2004). Algorithms for use with mapping assays are described, for example, in Liu et al., Bioinformatics. 19: 2397-2403 (2003) and Di et al. Bioinformatics. 21:1958 (2005). Additional methods related to WGSA and arrays useful for WGSA and applications of WGSA are disclosed, for example, in U.S. Patent Application Nos. 60/676,058 filed Apr. 29, 2005, 60/616,273 filed Oct. 5, 2004, Ser. Nos. 10/912,445, 11/044,831, 10/442,021, 10/650,332 and 10/463,991. Genome wide association studies using mapping assays are described in, for example, Hu et al., Cancer Res.; 65(7):2542-6 (2005), Mitra et al., Cancer Res., 64(21):8116-25 (2004), Butcher et al., Hum Mol Genet., 14(10):1315-25 (2005), and Klein et al., Science. 308(5720):385-9 (2005).

Additionally, certain embodiments may include methods for providing genetic information over networks such as the Internet as shown, for example, in U.S. application Ser. Nos. 10/197,621, 10/063,559 (United States Publication Number 2002/0183936), Ser. Nos. 10/065,856, 10/065,868, 10/328,818, 10/328,872, 10/423,403, and 60/482,389.

### Pharmaceutical Formulations, Administration, and Kits

Embodiments of the present invention include GlyRS polypeptides, antibodies, binding agents, neuropilins or other compounds described herein, formulated in pharmaceutically-acceptable or physiologically-acceptable solutions for administration to a cell or an animal, either alone, or in combination with one or more other modalities of therapy. It will also be understood that, if desired, the compositions of the invention may be administered in combination with other agents as well, such as, e.g., other proteins or polypeptides or various pharmaceutically-active agents. There is virtually no limit to other components that may also be included in the compositions, provided that the additional agents do not adversely affect the modulatory or other effects desired to be achieved.

In the pharmaceutical compositions of the invention, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

In certain applications, the pharmaceutical or therapeutic compositions of the invention do not stimulate an immune reaction. In other embodiments, the pharmaceutical or therapeutic compositions of the invention, typically comprising one or more GlyRS polypeptides or polynucleotides, antibodies, binding agents and/or small molecules, stimulate an immune reaction, such as by serving as an adjuvant in a vaccine or related composition, or being present in a composition together with a separate adjuvant or agent stimulates an immune response.

In certain embodiments, the antibodies, binding agents, and other compounds described herein have a solubility that is desirable for the particular mode of administration, such intravenous administration. Examples of desirable solubilities include at least about 1 mg/ml, at least about 10 mg/ml, at least about 25 mg/ml, and at least about 50 mg/ml.

In certain applications, the pharmaceutical compositions disclosed herein may be delivered via oral administration to a subject. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain circumstances it will be desirable to deliver the pharmaceutical compositions disclosed herein parenterally, intravenously, intramuscularly, or even intraperitoneally as described, for example, in U.S. Pat. No. 5,543,158; U.S. Pat. No. 5,641,515 and U.S. Pat. No. 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions can be prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the pharmaceutical compositions may be delivered by intranasal sprays, inhalation, and/or other aerosol delivery vehicles. Methods for delivering genes, polynucleotides, and peptide compositions directly to the lungs via nasal aerosol sprays have been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

In certain embodiments, the agents provided herein may be attached to a pharmaceutically acceptable solid substrate, including biocompatible and biodegradable substrates such as polymers and matrices. Examples of such solid substrates include, without limitation, polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as poly(lactic-co-glycolic acid) (PLGA) and the LUPRON DEPOT™ (injectable microspheres composed of lactic acid-glycolic acid copolymer and leuprolide acetate), poly-D-(−)-3-hydroxybutyric acid, collagen, metal, hydroxyapatite, bioglass, aluminate, bioceramic materials, and purified proteins.

In one particular embodiment, the solid substrate comprises ATRIGEL® (QLT, Inc., Vancouver, B.C.). The ATRIGEL® drug delivery system consists of biodegradable polymers dissolved in biocompatible carriers. Pharmaceuticals may be blended into this liquid delivery system at the time of manufacturing or, depending upon the product, may be added later by the physician at the time of use. When the liquid product is injected into the subcutaneous space through a small gauge needle or placed into accessible tissue sites through a cannula, water in the tissue fluids causes the polymer to precipitate and trap the drug in a solid implant. The drug encapsulated within the implant is then released in a controlled manner as the polymer matrix biodegrades with time.

Methods of formulation are well known in the art and are disclosed, for example, in Remington: The Science and Practice of Pharmacy, Mack Publishing Company, Easton, Pa., 19th Edition (1995). The compositions and agents provided herein may be administered according to the methods of the present invention in any therapeutically effective dosing regime. The dosage amount and frequency are selected to create an effective level of the agent without harmful effects. The effective amount of a compound of the present invention will depend on the route of administration, the type of warm-blooded animal being treated, and the physical characteristics of the specific warm-blooded animal under consideration. These factors and their relationship to determining this amount are well known to skilled practitioners in the medical arts. This amount and the method of administration can be tailored to achieve optimal efficacy but will depend on such factors as weight, diet, concurrent medication and other factors which those skilled in the medical arts will recognize.

In particular embodiments, the amount of a composition or agent administered will generally range from a dosage of from about 0.1 to about 100 mg/kg/day, and typically from about 0.1 to 10 mg/kg where administered orally or intravenously. In particular embodiments, a dosage is 5 mg/kg or 7.5 mg/kg. In various embodiments, the dosage is about 50-2500 mg per day, 100-2500 mg/day, 300-1800 mg/day, or 500-1800 mg/day. In one embodiment, the dosage is between about 100 to 600 mg/day. In another embodiment, the dosage is between about 300 and 1200 mg/day. In particular embodiments, the composition or agent is administered at a dosage of 100 mg/day, 240 mg/day 300 mg/day, 600 mg/day, 1000 mg/day, 1200 mg/day, or 1800 mg/day, in one or more doses per day (i.e., where the combined doses achieve the desired daily dosage). In related embodiments, a dosage is 100 mg bid, 150 mg bid, 240 mg bid, 300 mg bid, 500 mg bid, or 600 mg bid. In various embodiments, the composition or agent is administered in single or repeat dosing. The initial dosage and subsequent dosages may be the same or different.

In certain embodiments, a composition or agent is administered in a single dosage of 0.1 to 10 mg/kg or 0.5 to 5 mg/kg. In other embodiments, a composition or agent is administered in a dosage of 0.1 to 50 mg/kg/day, 0.5 to 20 mg/kg/day, or 5 to 20 mg/kg/day.

In certain embodiments, a composition or agent is administered orally or intravenously, e.g., by infusion over a period of time of about, e.g., 10 minutes to 90 minutes. In other related embodiments, a composition or agent is administered by continuous infusion, e.g., at a dosage of between about 0.1 to about 10 mg/kg/hr over a time period. While the time period can vary, in certain embodiments the time period may be between about 10 minutes to about 24 hours or between about 10 minutes to about three days.

In particular embodiments, an effective amount or therapeutically effective amount is an amount sufficient to achieve a total concentration of the composition or agent in the blood plasma of a subject with a Cₘₐₓ of between about 0.1 μg/ml and about 20 μg/ml or between about 0.3 μg/ml and about 20 μg/ml. In certain embodiments, an oral dosage is an amount sufficient to achieve a blood plasma concentration (Cₘₐₓ) of between about 0.1 μg/ml to about 5 μg/ml or between about 0.3 μg/ml to about 3 μg/ml. In certain embodiments, an intravenous dosage is an amount sufficient to achieve a blood plasma concentration (Cₘₐₓ) of between about 1 μg/ml to about 10 μg/ml or between about 2 μg/ml and about 6 μg/ml. In a related embodiment, the total concentration of an agent in the blood plasma of the subject has a mean trough concentration of less than about 20 μg/ml and/or a steady state concentration of less than about 20 μg/ml. In a further embodiment, the total concentration of an agent in the blood plasma of the subject has a mean trough concentration of less than about 10 μg/ml and/or a steady state concentration of less than about 10 μg/ml.

In yet another embodiment, the total concentration of an agent in the blood plasma of the subject has a mean trough concentration of between about 1 ng/ml and about 10 μg/ml and/or a steady state concentration of between about 1 ng/ml and about 10 μg/ml. In one embodiment, the total concentration of an agent in the blood plasma of the subject has a mean trough concentration of between about 0.3 μg/ml and about 3 μg/ml and/or a steady state concentration of between about 0.3 μg/ml and about 3 μg/ml.

In particular embodiments, a composition or agent is administered in an amount sufficient to achieve in the mammal a blood plasma concentration having a mean trough concentration of between about 1 ng/ml and about 10 μg/ml and/or a steady state concentration of between about 1 ng/ml and about 10 μg/ml. In related embodiments, the total concentration of the agent in the blood plasma of the mammal has a mean trough concentration of between about 0.3 μg/ml and about 3 μg/ml and/or a steady state concentration of between about 0.3 μg/ml and about 3 μg/ml.

In particular embodiments of the present invention, the effective amount of a composition or agent, or the blood plasma concentration of composition or agent is achieved or maintained, e.g., for at least 15 minutes, at least 30 minutes, at least 45 minutes, at least 60 minutes, at least 90 minutes, at least 2 hours, at least 3 hours, at least 4 hours, at least 8 hours, at least 12 hours, at least 24 hours, at least 48 hours, at least 3 days, at least 4 days, at least 5 days, at least 6 days, at least one week, at least 2 weeks, at least one month, at least 2 months, at least 4 months, at least 6 months, at least one year, at least 2 years, or greater than 2 years.

In certain polypeptide-based embodiments, the amount of polypeptide administered will typically be in the range of about 0.1 μg/kg to about 0.1 mg/kg to about 50 mg/kg of patient body weight. Depending on the type and severity of the disease, about 0.1 μg/kg to about 0.1 mg/kg to about 50 mg/kg body weight (e.g., about 0.1-15 mg/kg/dose) of polypeptide can be an initial candidate dosage for administration to the patient, whether, for example, by one or more separate administrations, or by continuous infusion. For example, a dosing regimen may comprise administering an initial loading dose of about 4 mg/kg, followed by a weekly maintenance dose of about 2 mg/kg of the polypeptide, or about half of the loading dose. However, other dosage regimens may be useful. A typical daily dosage might range from about 0.1 μg/kg to about 1 μg/kg to 100 mg/kg or more, depending on the factors mentioned above. For repeated administrations over several days or longer, depending on the condition, the treatment is sustained until a desired suppression of disease symptoms occurs.

In particular embodiments, the effective dosage achieves the blood plasma levels or mean trough concentration of a composition or agent described herein. These may be readily determined using routine procedures.

Embodiments of the present invention, in other aspects, provide kits comprising one or more containers filled with one or more of the polypeptides, polynucleotides, antibodies, multiunit complexes, compositions thereof, etc., of the invention, as described herein. The kits can include written instructions on how to use such compositions (e.g., to modulate cellular signaling, angiogenesis, cancer, inflammatory conditions, diagnosis etc.).

The kits herein may also include a one or more additional therapeutic agents or other components suitable or desired for the indication being treated, or for the desired diagnostic application. An additional therapeutic agent may be contained in a second container, if desired. Examples of additional therapeutic agents include, but are not limited to anti-neoplastic agents, anti-inflammatory agents, antibacterial agents, antiviral agents, angiogenic agents, etc.

The kits herein can also include one or more syringes or other components necessary or desired to facilitate an intended mode of delivery (e.g., stents, implantable depots, etc.).

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### HDX Analysis to Identify Opened-Up Regions in Glycyl-tRNA Synthetases Associated with Charcot-Marie-Tooth (CMT) Disease

The question of how dispersed mutations in one protein engender the same gain-of-function phenotype was explored. The studies described herein focused on mutations in glycyl-tRNA synthetase (GlyRS) which cause an axonal form of Charcot-Marie-Tooth (CMT) diseases, the most common hereditary peripheral neuropathies. Because the disease phenotype is dominant, and not correlated with defects in the role of GlyRS in protein synthesis, the mutant proteins are considered to be gain-of-function neomorphs.

Given that previous crystal structures showed little conformational difference between dimeric wild-type and CMT-causing mutant GlyRSs, the mutant proteins were investigated in solution by hydrogen-deuterium exchange (monitored by mass spectrometry) and small-angle X-ray scattering to uncover structural changes that could be suppressed by crystal packing interactions. As discussed below, each of 5 spatially dispersed mutations induced the same conformational opening of a consensus area that is mostly buried in the wild-type protein. The identified neomorphic surface(s) are thus a candidate for making CMT-associated pathological interactions, and a target for disease correction. Additional results showed that a WHEP domain that was appended to GlyRS in metazoans can regulate the neomorphic structural change, and that the gain of function of the CMT mutants might due to the loss of function of the WHEP domain as a regulator. Overall, the results described herein demonstrate how spatially dispersed and seemingly unrelated mutations can perpetrate the same localized effect on a protein.

The structures of five different CMT-causing mutant proteins were explored in solution by use of hydrogen-deuterium exchange analysis monitored by mass spectrometry (HDX MS). HDX MS determines the solvent exposure of individual peptide segments throughout a protein. By annotating the exposure of the same peptide segments in the WT and mutant proteins, a map of structural differences can be constructed. In addition, small-angle X-ray scattering (SAXS) was used to study protein shape changes in solution. These approaches are able to avoid the problems of crystal packing interactions suppressing conformational differences that existed in solution. Surprisingly, it was found that each of the mutations tested induced a structural opening that was mapped to specific but common regions of the protein.

### Materials and Methods

Analytical Ultracentrifugation.

WT and mutant GlyRSs were cloned and expressed as described (see Xie et al., Acta Crystallogr Sect F Struct Biol Cryst Commun 62:1243-1246, 2006), and purified to more than 99% homogeneity in three steps: nickel affinity, Mono Q and size exclusion chromatography. Sedimentation equilibrium experiments were performed in a Beckman Optima XL-1 analytical ultracentrifuge with integrated optical systems. All protein samples (0.5 μM, 1.25 μM, 2.5 μM) were prepared in DPBS buffer (2.67 mM potassium chloride, 1.47 mM potassium phosphate monobasic, 137.93 mM sodium chloride and 8.06 mM sodium phosphate dibasic, pH 7.2) and purified by size exclusion chromatography immediately prior to centrifugation. The buffer density and viscosity, and the protein partial specific volume were determined by SEDPHAT (see Lebowitz et al., Protein Sci. 11:2067-2079, 2002). Samples (180 μL) in two-channel cells were centrifuged in a Beckman An-60 Ti rotor at speeds of 9000, 14000, 17000 rpm at 4° C. Equilibrium was reached after 36 hours for each speed and verified by comparison with a second scan taken every 6 hours. Data was collected at 280 nm and 230 nM. Data sets consisting of 18 curves per protein (3 concentrations, 3 rotor speeds and 2 wavelengths each) were analyzed by global fitting to the monomer-dimer self-association model using the program SEDPHAT (Id.).

HDX Fourier Transform Ion Cyclotron Resonance (FT-ICR) Mass Spectrometry.

The HDX MS procedure was automated with a leap robot (HTS PAL, Leap Technologies, Carrboro, N.C.) as described (see Zhang et al., Anal Chem 80:9034-9041, 2008). Briefly, 5 μL of GlyRS protein (WT or mutant) was mixed with 45 μL of DPBS buffer in D₂O (2.6 μM final concentration) to initiate HDX reaction for 0.5, 1, 2, 4, 8, 15, 30, 60, 120 or 240 minutes, followed by simultaneous quenching and proteolysis by adding protease type XIII solution (3-fold diluted saturated solution) in 1.0% formic acid, 40 mM TCEP and 4 M urea (see Zhang et al., Anal Chem 82:1450-1454, 2010). The digested peptide fragments were separated by a fast LC gradient through a ProZap C₁₈ column (Grace Davidson, 1.5 μm, 500 Å, 2.1×10 mm) to minimize back exchange (see Zhang et al., J Am Soc Mass Spectrom 20:520-524, 2009). A post-column splitter reduced the LC eluent flow rate to ˜400-500 nL/min for efficient microelectrospray ionization (micro-ESI) (see Emmett et al., J Am Soc Mass Spectrom 9:333-340, 1998). Microelectrosprayed HDX samples were directed to a custom-built hybrid linear trap quadrupole 14.5 tesla FT-ICR mass spectrometer (ThermoFisher, San Jose, Calif.) for accurate mass measurement (see Schaub et al., Anal Chem 80:3985-3990, 2008). The total data acquisition period for each sample was 6 minutes. All experiments were performed in triplicate. Data were analyzed with a custom analysis package (see Kazazic et al., J Am Soc Mass Spectrom 21:550-558, 2010) and a Python program (see Zhang et al., Protein Sci 19:703-715, 2010). Time-course deuterium incorporation levels were generated by an MEM fitting method (Zhang et al., Protein Sci 6:2203-2217, 1997).

SAXS.

SAXS data for WT and G526R GlyRS were collected at the 12.3.1 beamline of the Advanced Light Source (ALS, Berkeley) with an ADSC Quantum 315r detector. The measurements were performed at 20° C. at two protein concentrations (0.5 and 1.0 mg/mL). No systematic differences were detected between the two concentrations, indicating the absence of interparticle interference at those concentrations. Radiation damage on protein samples was monitored after two 0.5 s exposures, and no damage was observed. The average background scattering from the buffer, determined by two independent measurements (before and after each sample measurement), was subtracted from the scattering profile of the protein. The data were processed by standard procedures with the program PRIMUS (see Konarev et al., J Appl Crystallogr 36:1277-1282, 2003). The forward scattering I(0) and the radii of gyration R_{g} were estimated from the Guinier approximation under the assumption that at very small angle (s<1.3/Rd the intensity is represented as I(s)=I(0)exp(−(sR_{g})²/3). Maximum particle dimension Dₘₐₓ were computed by use of the indirect transform package GNOM (see Konig et al., Biochemistry 31:8726-8731, 1992), which also gave the distance distribution function p(r).

Ab initio free atom modeling was performed with the program DAMMIF (see Franke and Svergun, J Appl Crystallogr 42:342-346, 2009). Ten independent simulations were carried out for each protein. Subsequent superposition, averaging, and filtering with the DAMAVER program (see Volkov et al., J Appl Crystallogr 36:860-864., 2003) generated the final shape reconstructions.

### Results

It was first observed that CMT mutations had an idiosyncratic effect on dimer-monomer equilibrium. GlyRS functions as a dimer for aminoacylation. Amino acid substitutions that are located on the dimer interface are likely to affect the dimer-monomer equilibrium. Indeed, using co-immunoprecipitation to detect heterodimer formation in vivo between transfected mutant GlyRS and endogenous WT GlyRS, it had been previously shown that different mutations have different effects on dimer formation (Nangle et al., PNAS USA 104:11239-11244, 2007). For example, L129P and G240R have a greatly reduced capacity to form heterodimers with WT GlyRS. In contrast, S581L and G526R have an increased capacity for heterodimer formation.

In terms of homodimer formation, the opposite effects of the G240R and G526R mutations were also validated by the analytical ultracentrifugation (AUC) analysis. Two complementary views of solution behavior are accessible from AUC analyses. Sedimentation velocity (SV) provides hydrodynamic information about size and shape, whereas sedimentation equilibrium (SE) provides thermodynamic information about the molecule mass, stoichiometry of subunit assembly, and the association constant (see Howlett et al., Curr Opin Chem Biol 10:430-436, 2006). SV analysis in previous work showed that a larger population of monomer species was found with G240R relative to WT GlyRS, while the opposite was true for G526R (see Nangle et al and Xie et al., supra).

To obtain quantitative understanding of the effect of CMT-causing mutations on the monomer-dimer equilibrium, the dissociation constants of WT and mutant GlyRSs were measured by the SE method (see Table 1 and FIGS. 8A-8F). WT GlyRS had a K_{d} of 0.56 μM. Consistent with previous observations, L129P (K_{d}=41 μM) and G240R (K_{d}=11 μM) mutations cause 73- and 20-fold increases in K_{d} value, whereas S581L (K_{d}=0.19 μM) and G526R (K_{d}=0.15 μM) decreased the K_{d} about 3-4 fold. The K_{d} of G598A GlyRS was also determined; this mutant is associated with the most severe phenotype among all CMT 2D patients. Here, the G598A mutation had little effect on the monomer-dimer equilibrium (K_{d}=0.39 μM). These results indicate that the effect of a CMT-causing mutation on dimer stability is idiosyncratic.

HDX Analyses then Revealed Increased Deuterium Incorporation for all CMT-Associated Mutants.

In spite of their idiosyncratic effect on dimer stability, we wanted to understand if there is a shared conformational change induced by different mutations, given their common dimer interface localization. HDX MS is a powerful analytical tool to study protein dynamics and conformational changes in solution (see Zhang and Smith, Protein Sci 2:522-531, 1993; and Gajiwala, et al., PNAS USA 106:1542-1547, 2009). Backbone amide hydrogens in a protein are labile and will exchange with deuteriums when the protein is placed in a D₂O buffer. After each of a series of exchange periods, the exchange reaction is quenched by reducing the pH to ˜0.2.5, and the protein is proteolyzed into fragments and subjected to high-resolution mass spectrometry analysis (see Marshall et al., Mass Spectrom Rev 17:1-35, 1998). Because exchange of one hydrogen atom for deuterium results in one mass unit increase, deuterium incorporation of each peptide can be monitored by mass spectrometry. The rate and level of exchange are directly related to the local conformation of the peptide: solvent-accessible peptides exhibit faster and greater deuterium exchange than solvent-inaccessible ones.

The results for WT GlyRS are shown in FIG. 7A. According to K_{d} measurements for the monomer-dimer equilibrium, GlyRS was predominantly dimeric at the concentration for the HDX experiment (2.6 μM). Consistently, few peptides from the dimerization interface were associated with high deuterium uptake. In contrast, almost all peptides within the disordered regions in the crystal structure of WT GlyRS (e.g., the WHEP domain, Insertion III and the C-terminus) were associated with high deuterium uptake.

The results for the five mutant GlyRSs (L129P, G240R, G526R, S581L and G598A) are shown in Tables 1 and 2 below. Table 1 shows the dissociation constants of dimerization for WT and CMT-causing mutant GlyRSs, and the average increase in deuterium incorporation for each mutant relative to WT GlyRS. Table 2 shows the increase in deuterium incorporation (%) after 1 hour exchange at CMT-associated sites of each mutant relative to WT GlyRS.

TABLE 1

| | WT | L129P | G240R | G598A | S581L | G526R | ΔWHEP |
|---|---|---|---|---|---|---|---|
| K_{d} of dimerization | 0.56 μM | 41 μM | 11 μM | 0.39 μM | 0.19 μM | 0.16 μM | n.d. |
| HDX Increase* | 0% | 37% | 30% | 22% | 18% | 16% | 26% |

*The number is calculated from the deuterium incorporation difference after 1 h exchange for each proteolyzed peptide in a protein and then averaged for all of the peptides.

TABLE 2

| | A57 | E71 | L129 | C157 | P234 | G240 | P244 | I280 | H418 | D500 | G526 | S581 | G598 |
|---|---|---|---|---|---|---|---|---|---|---|---|---|---|
| L129P | 7 | 87 | 22 | 25 | 65 | 65 | 65 | 51 | n.c.* | n.c.* | 62 | n.c.* | 17 |
| G240R | 6 | 70 | n.c.* | 6 | 58 | 58 | 58 | 56 | n.c.* | 51 | 67 | n.c.* | 26 |
| G526R | 20 | 42 | 17 | n.c. | 8 | 8 | 8 | 28 | n.c.* | 38 | 26 | n.c.* | 14 |
| S581L | 12 | 27 | 27 | 33 | 38 | 38 | 38 | 37 | n.c.* | n.c.* | 38 | 16 | 16 |
| G598A | 20 | 30 | 26 | 14 | 56 | 56 | 56 | 37 | n.c.* | n.c.* | 43 | n.c.* | 30 |
| ΔWHEP | n.c.* | 56 | 20 | n.c.* | 55 | 55 | 55 | 37 | n.c.* | n.c.* | 35 | n.c.* | 20 |

*The residue is uncovered by HDX MS analysis for the mutant.

As shown in Table 1, the CMT-associated GlyRS mutants were distinctly different from WT GlyRS in terms of their dimer stability. Remarkably, all five mutations increased the overall deuterium incorporation of the protein (16%-37% over that of WT GlyRS after 1 hour exchange), suggesting a conformational opening induced by each mutation. The level of the conformational opening also correlates with the dimer stability: the less stable the mutant dimer, the higher increase in deuterium incorporation. However, even mutations that promote dimer association, such as G526R and S581L, increased deuterium uptake, suggesting that the CMT mutation-induced conformational opening is at least in part independent of dimer stability.

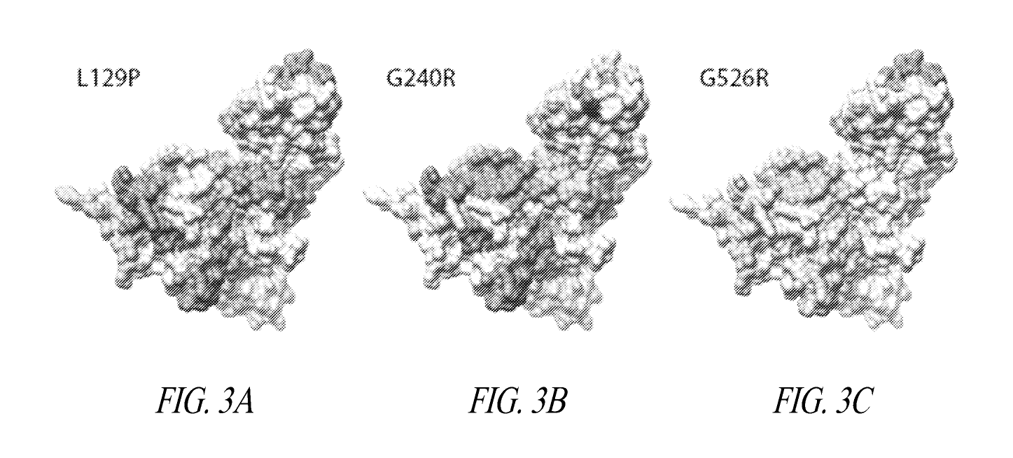

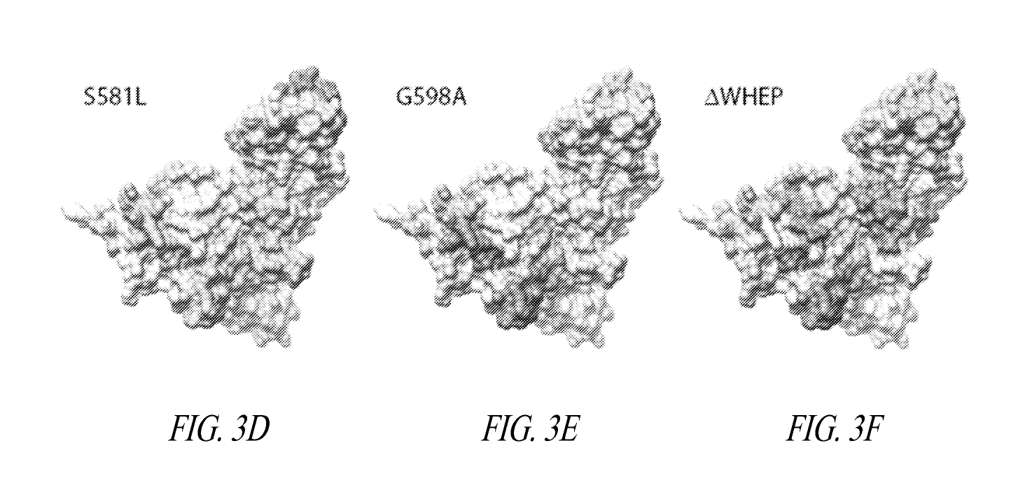

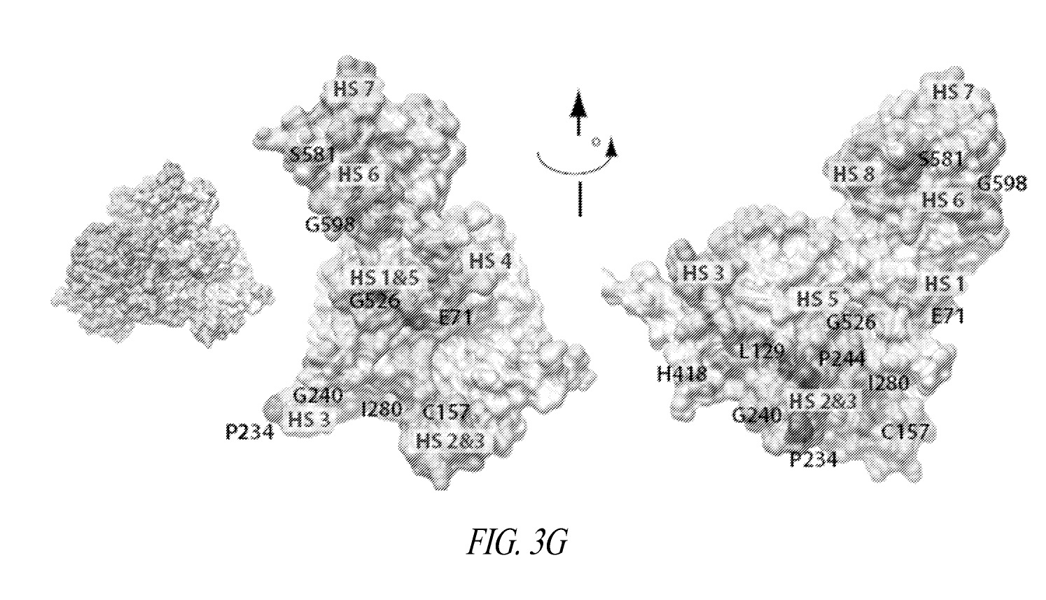

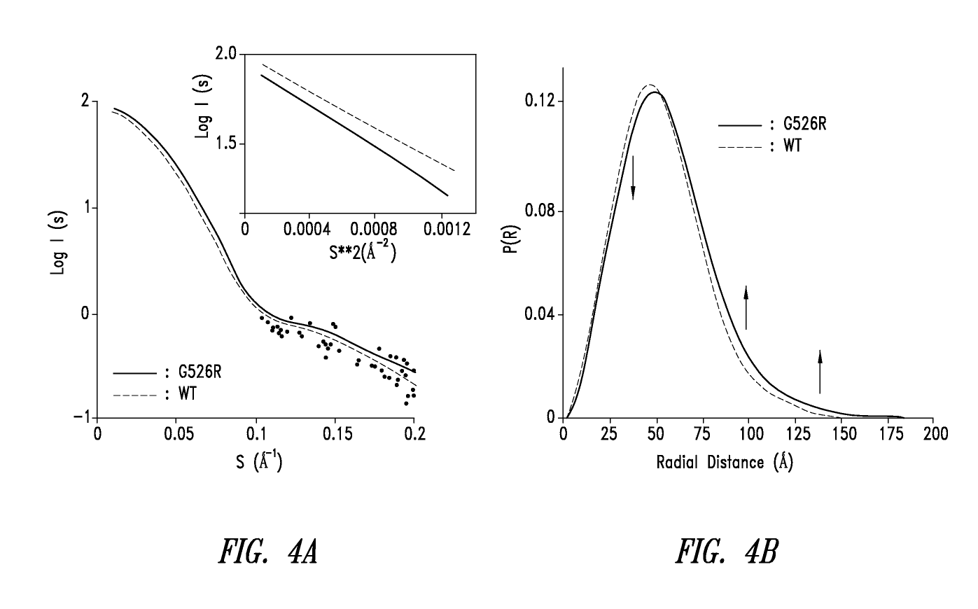

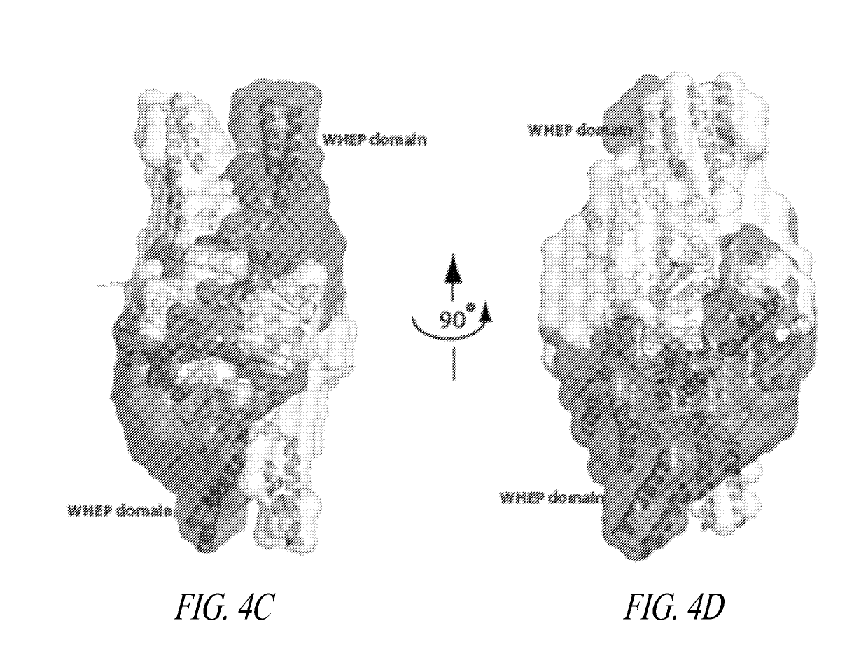

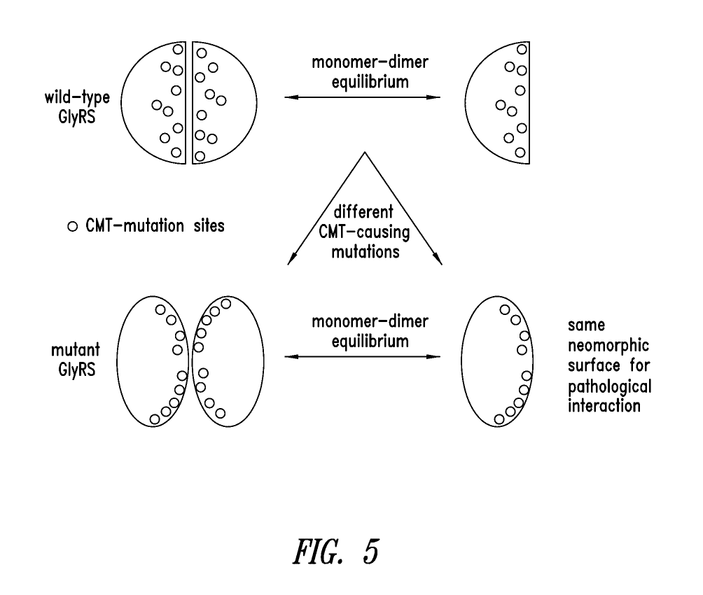

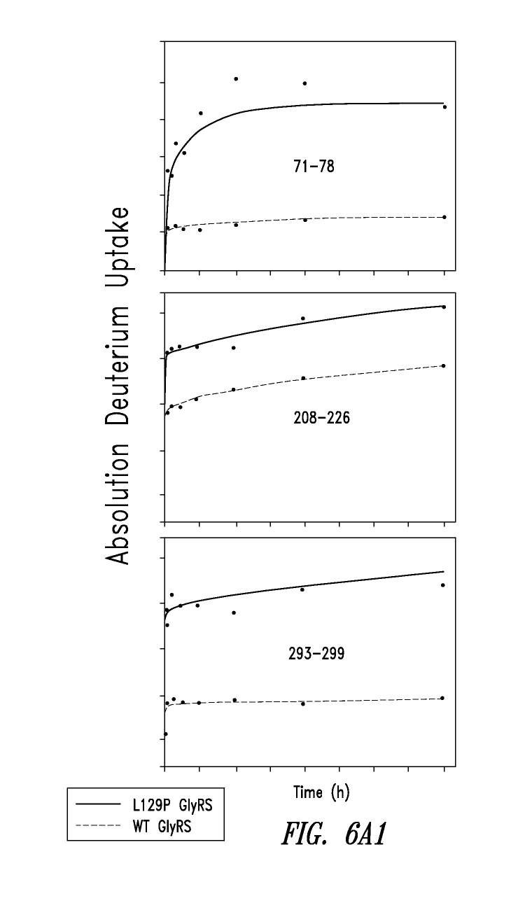

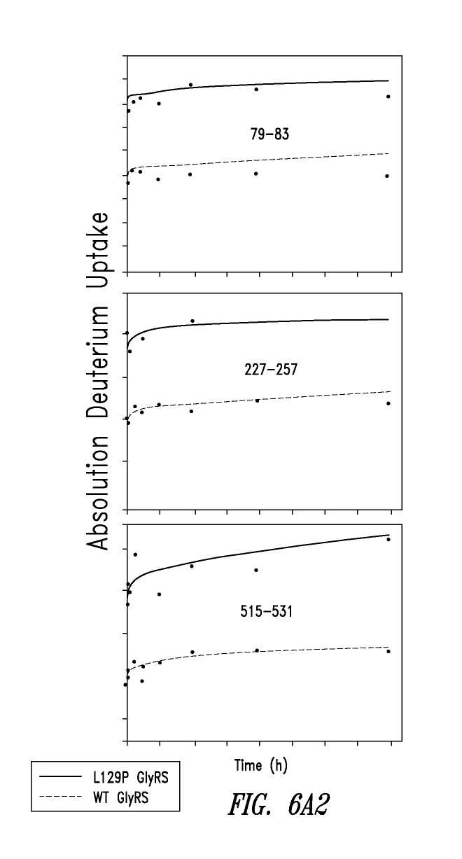

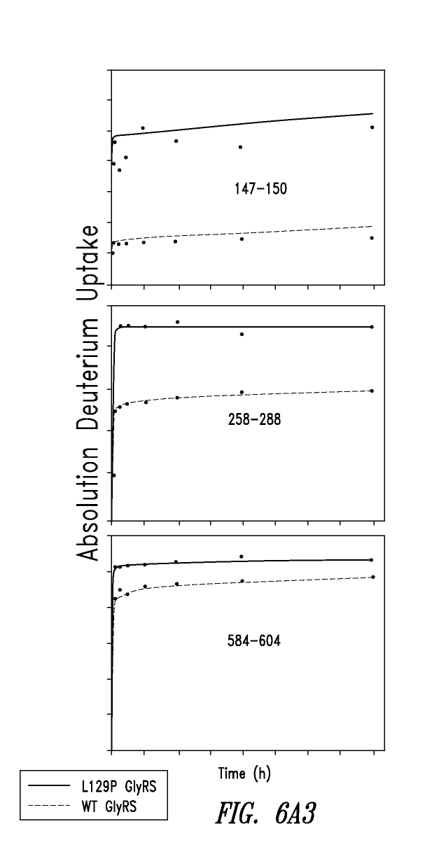

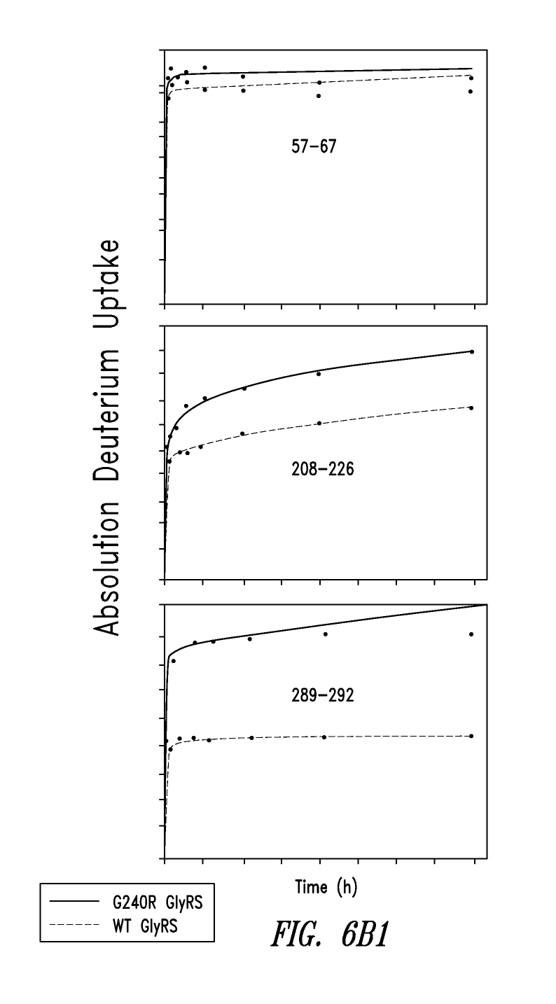

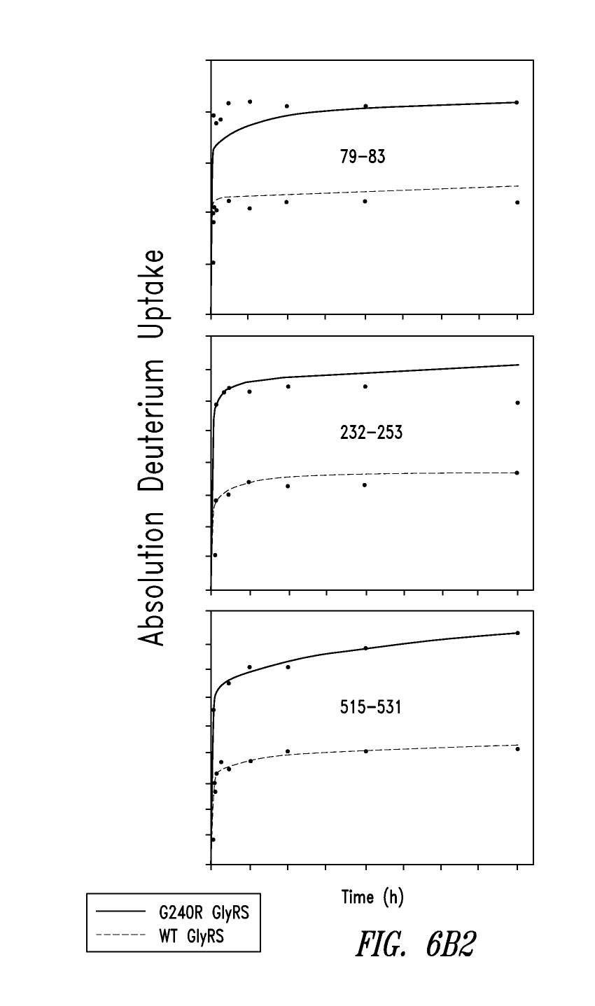

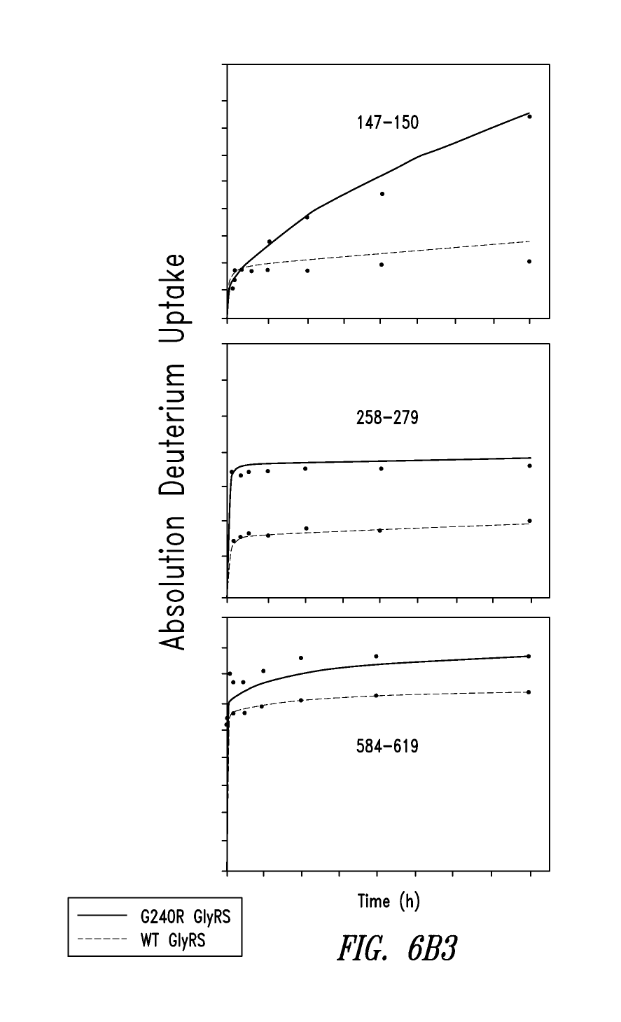

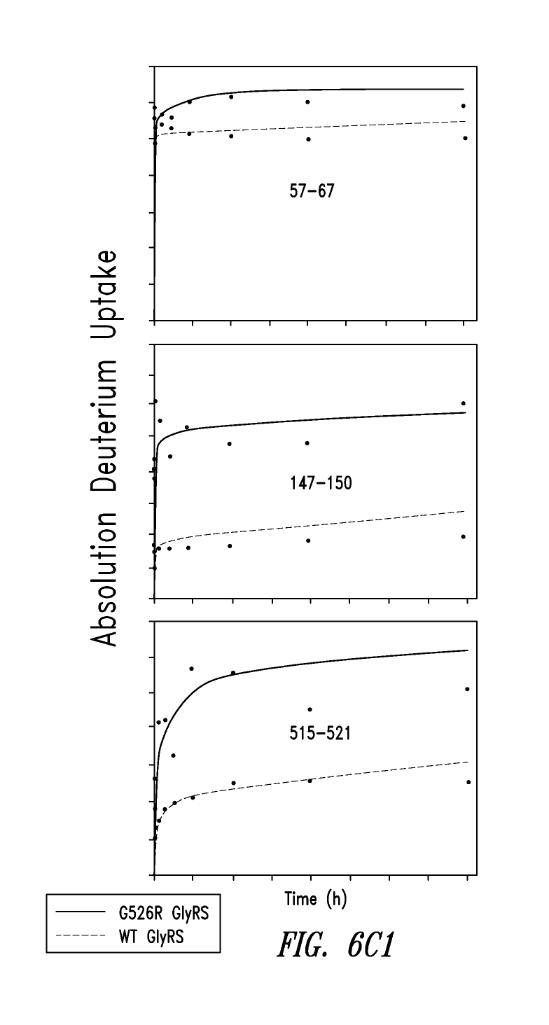

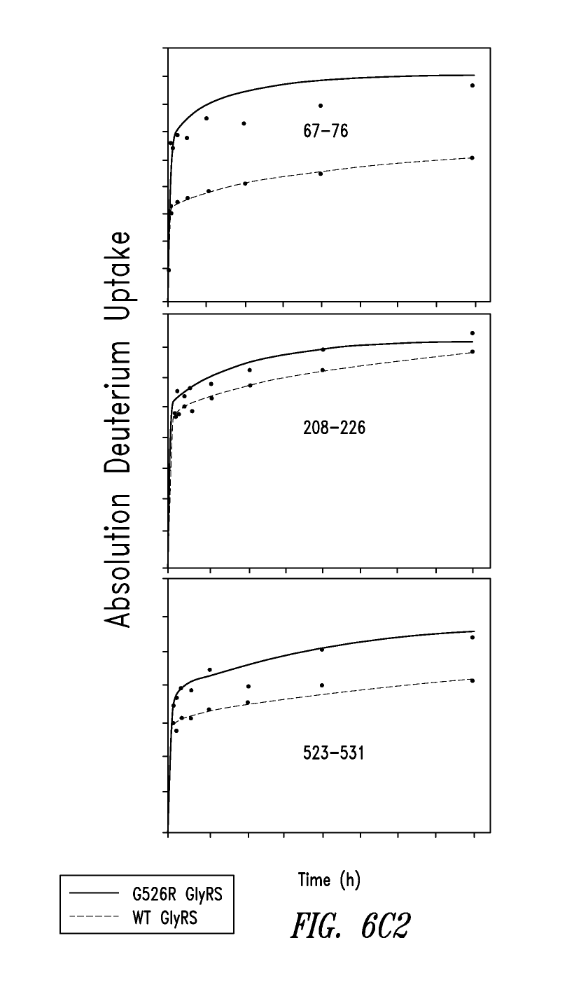

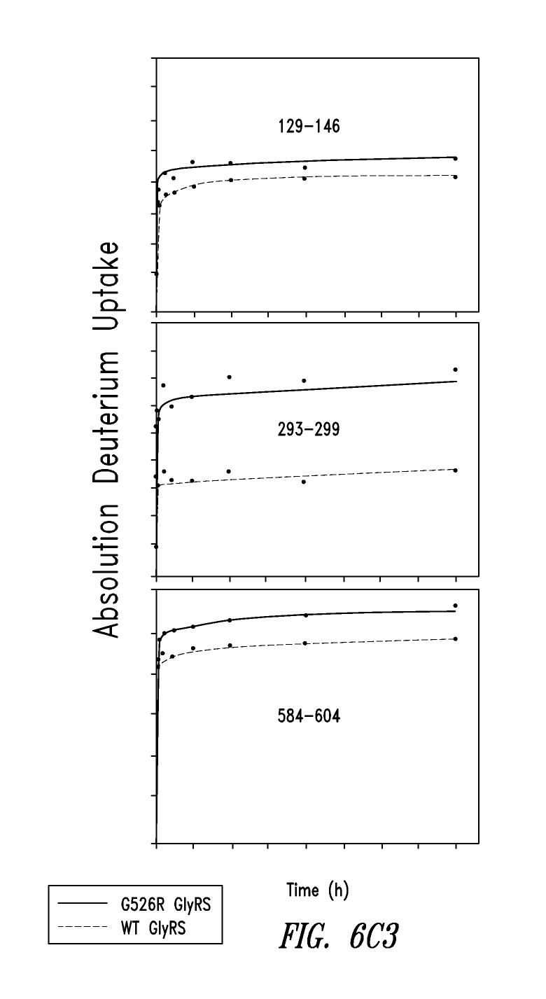

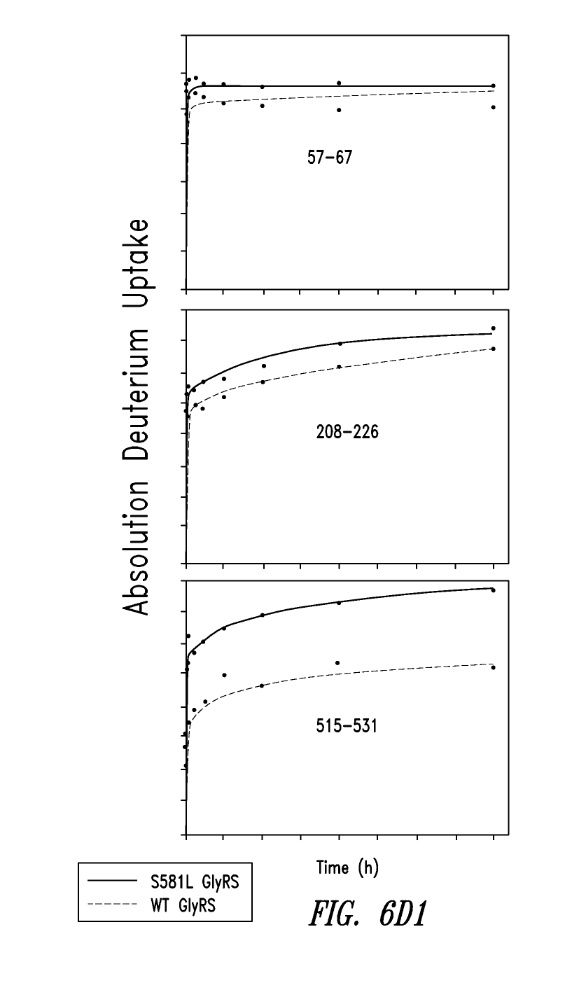

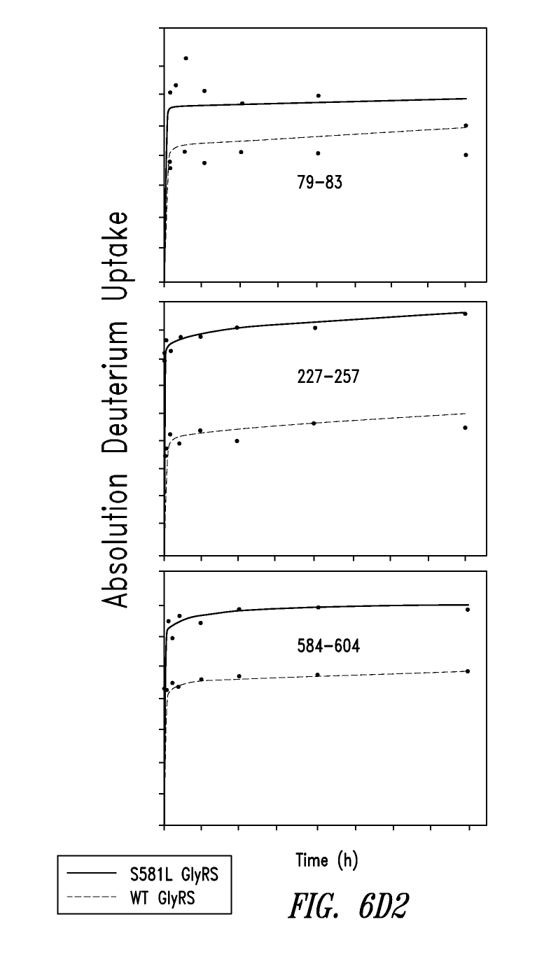

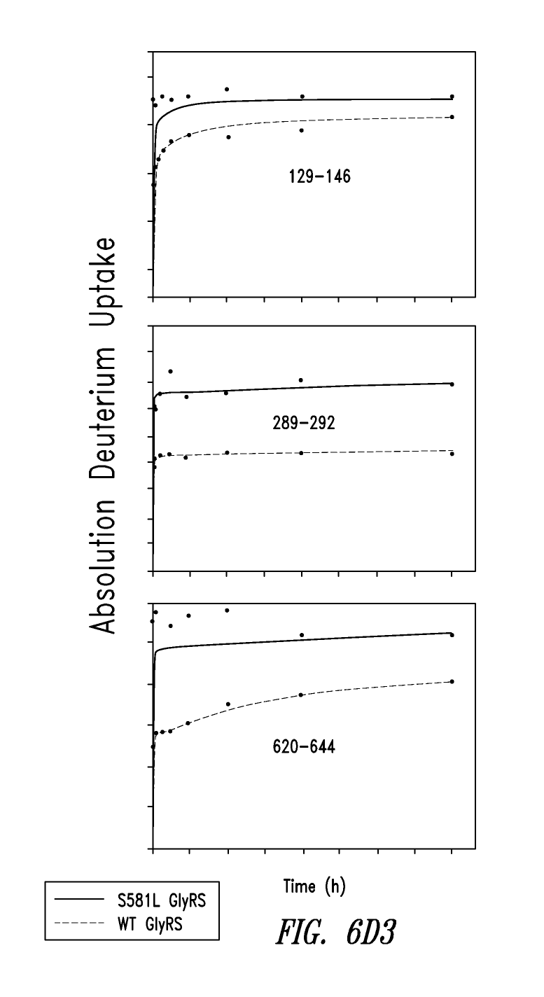

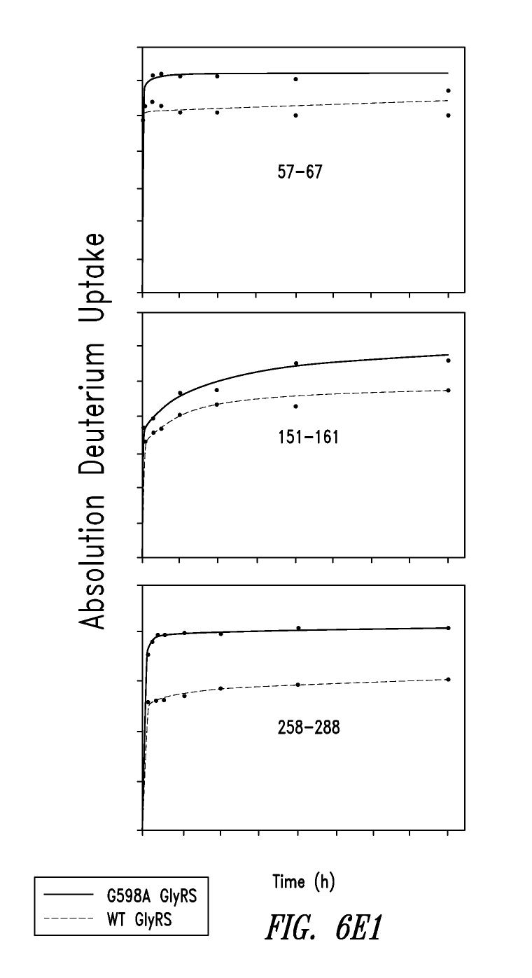

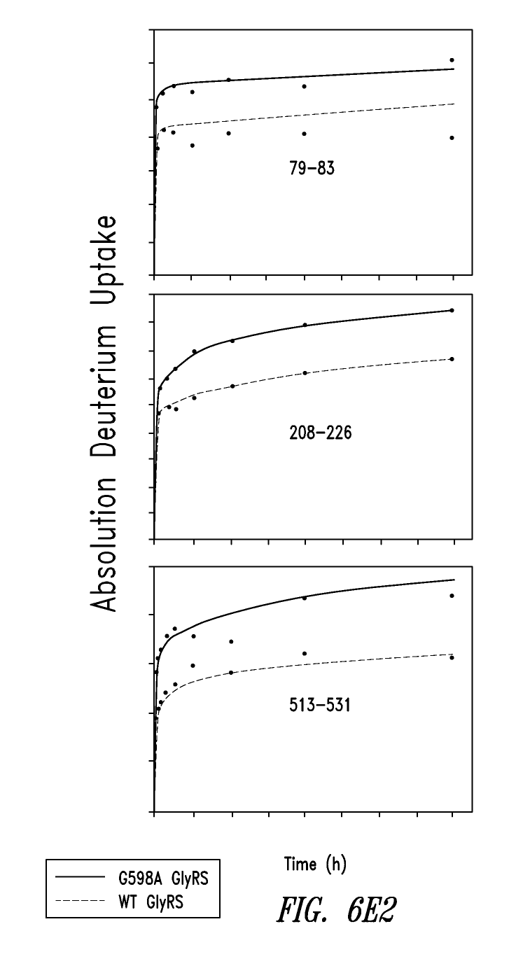

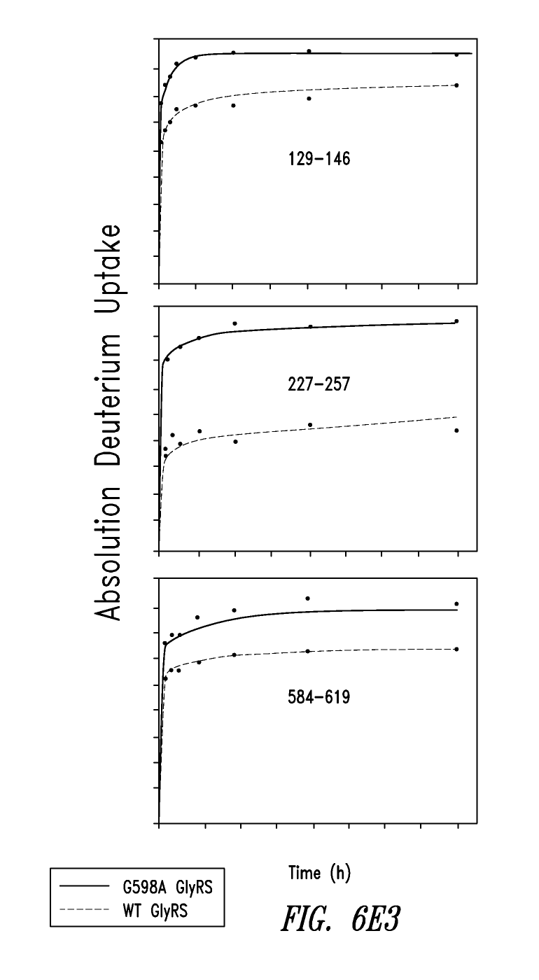

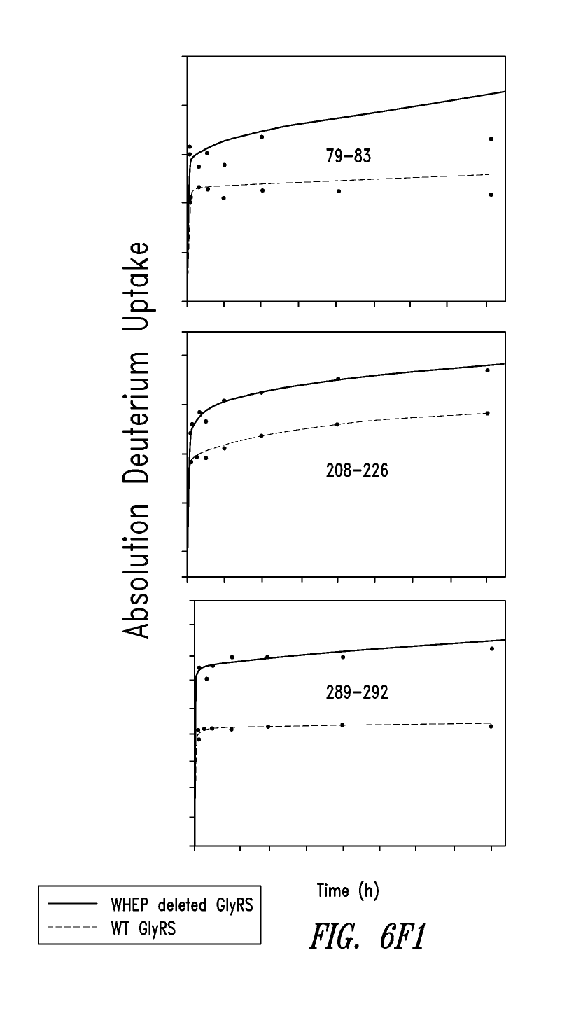

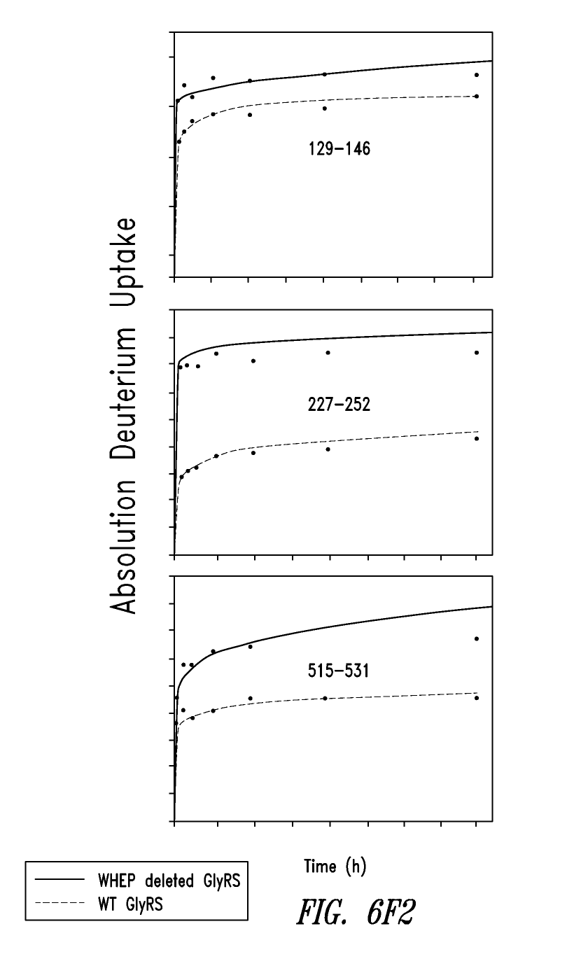

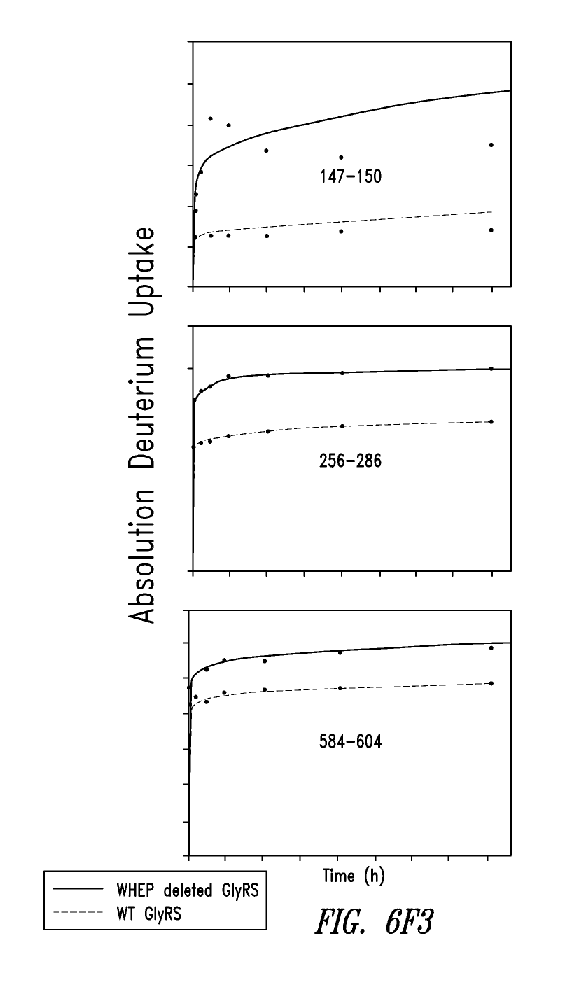

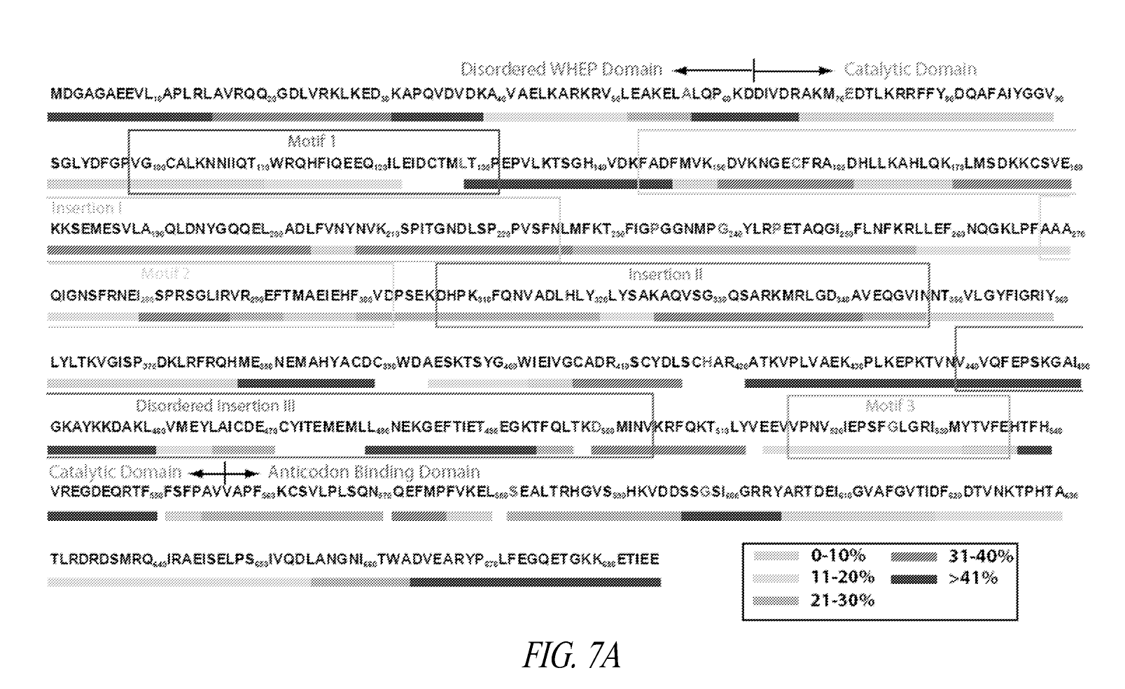

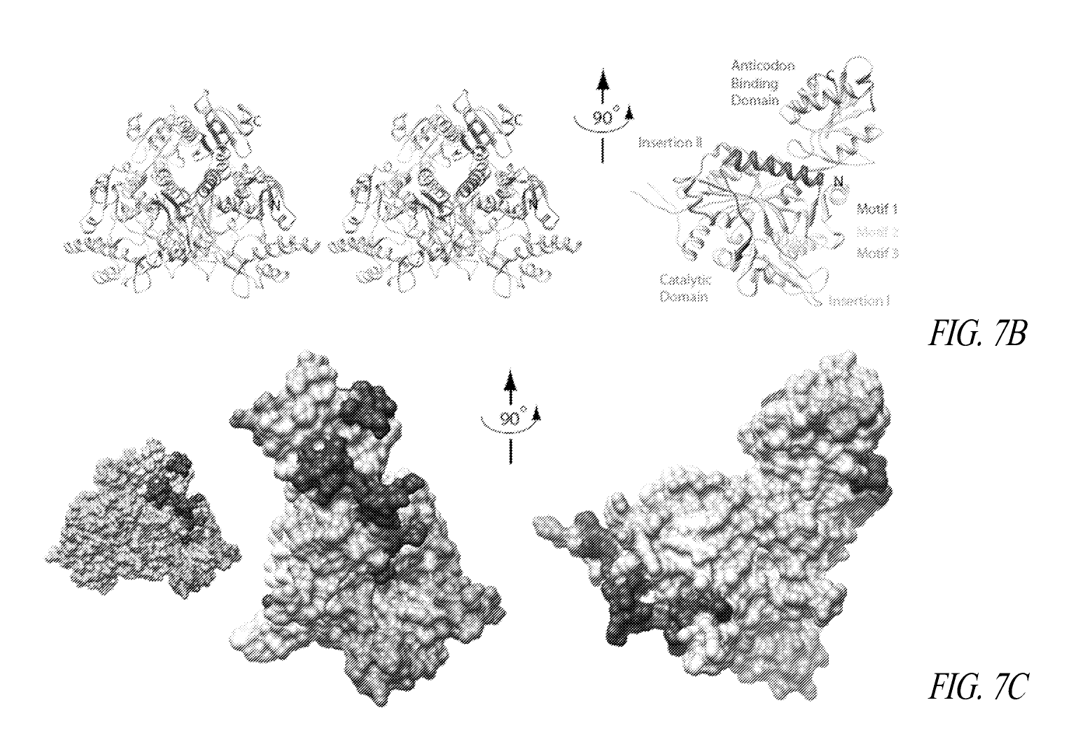

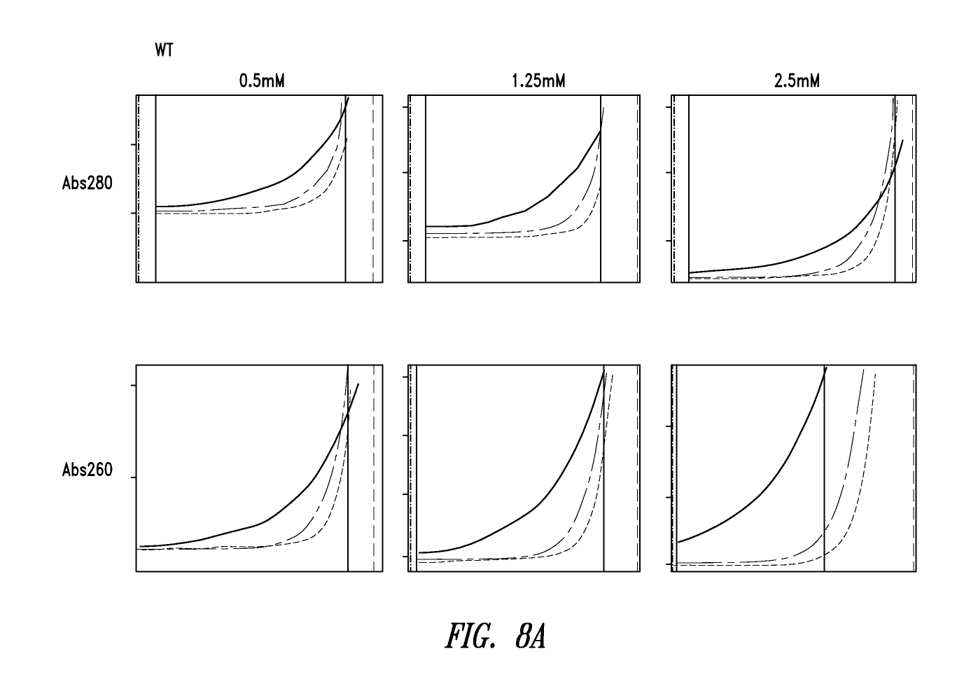

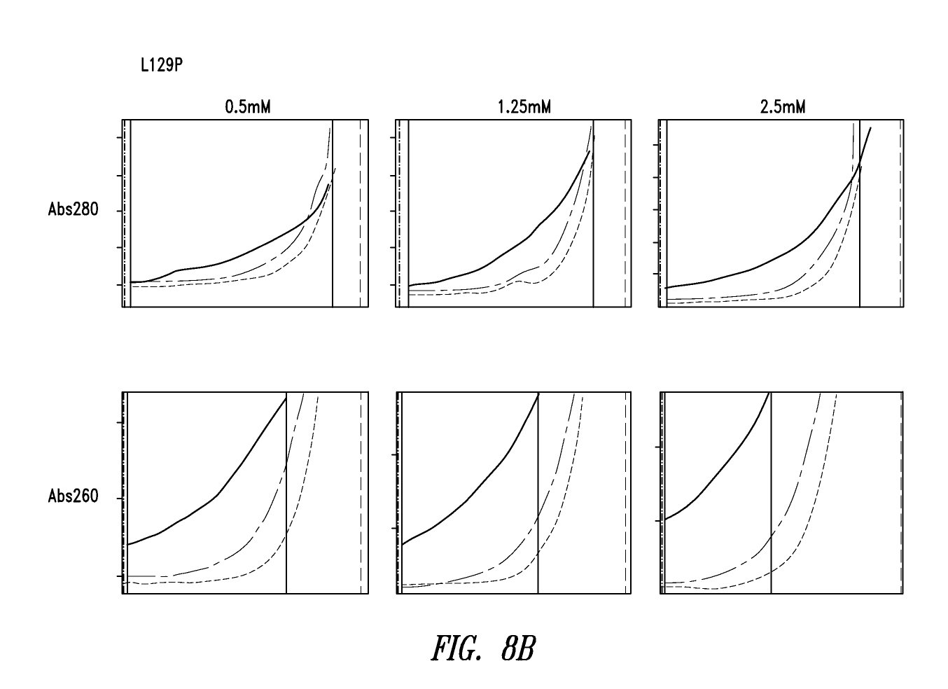

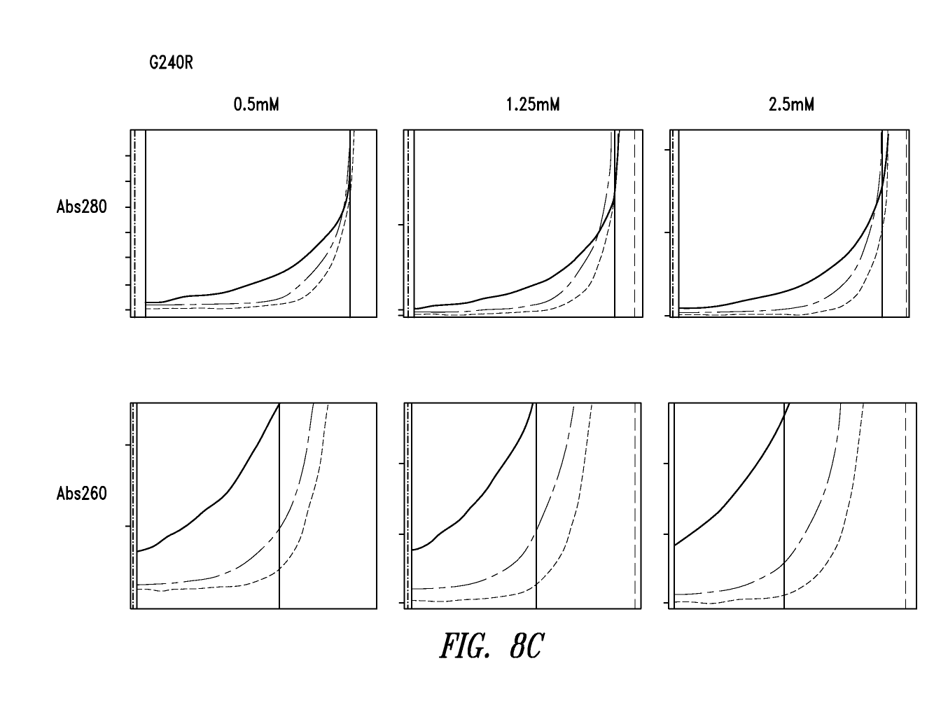

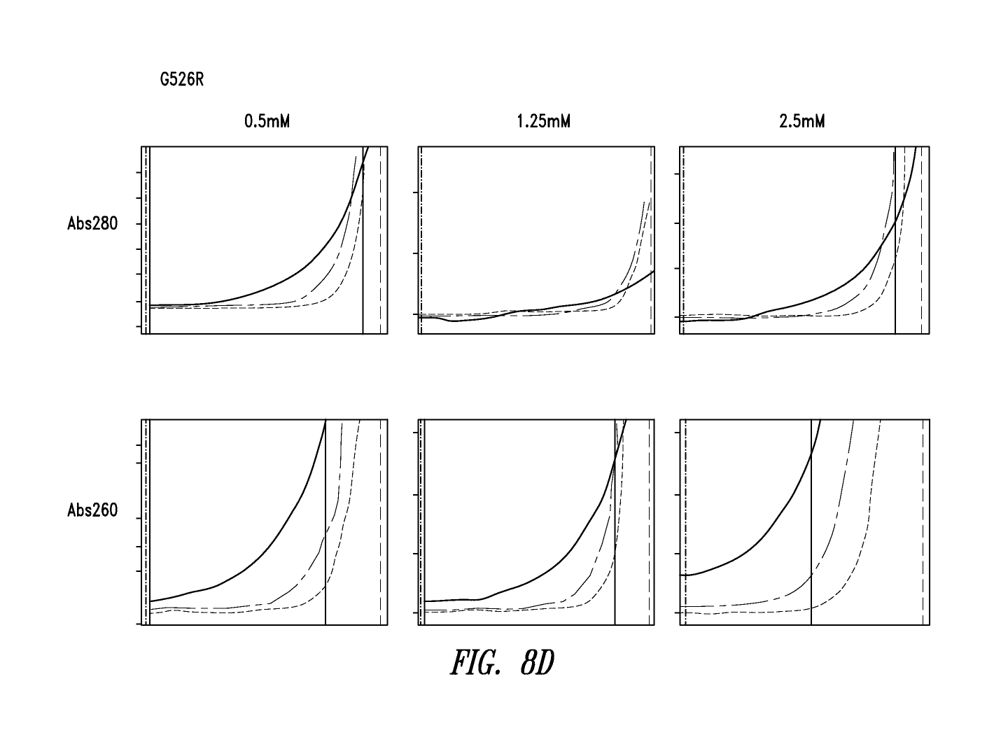

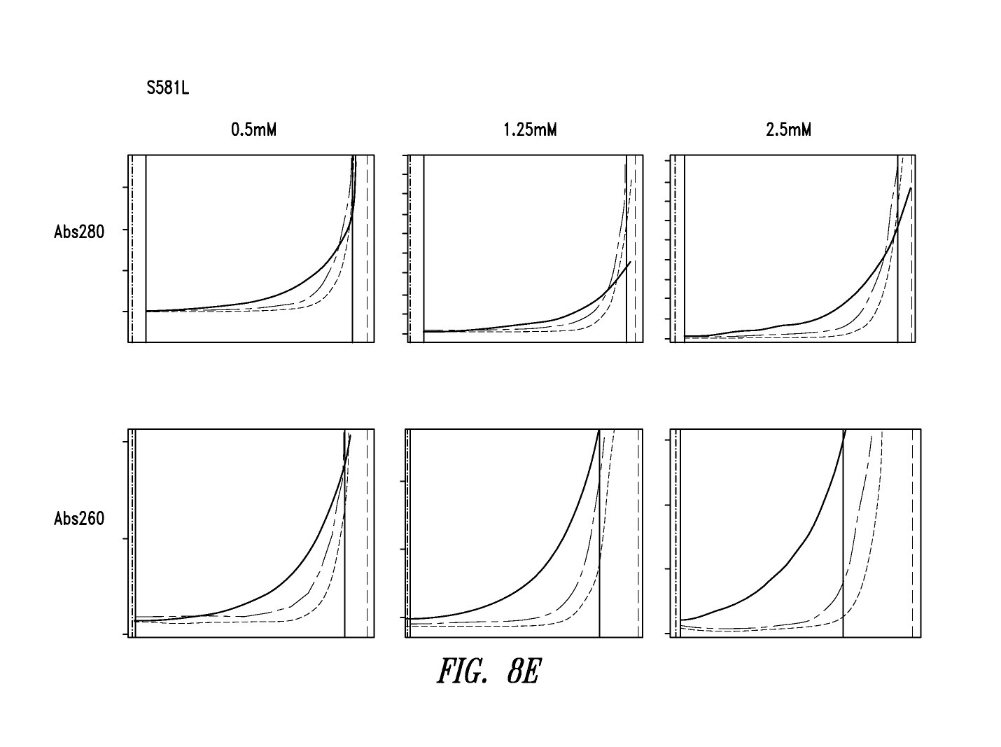

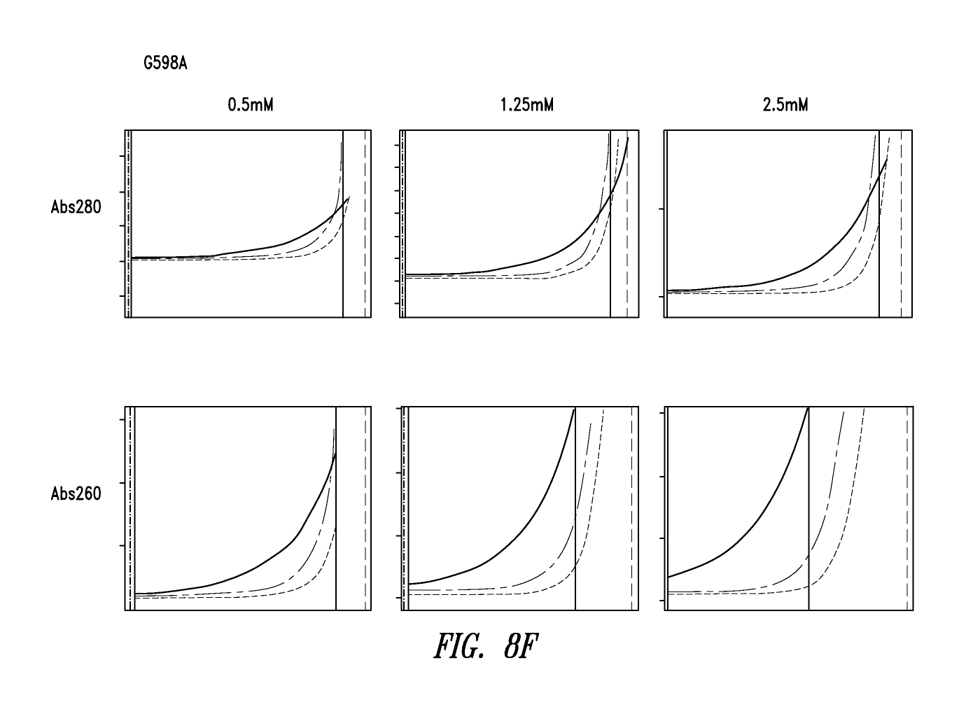

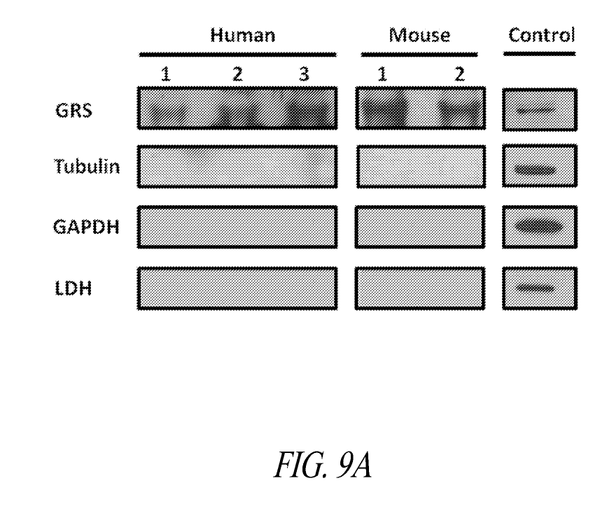

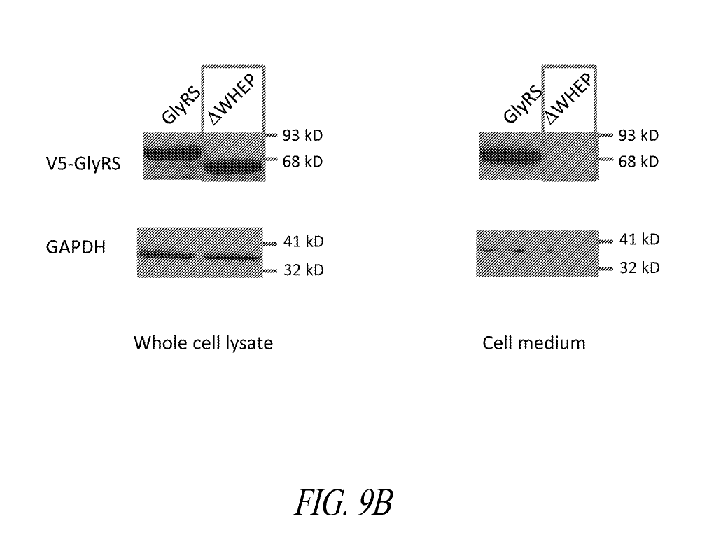

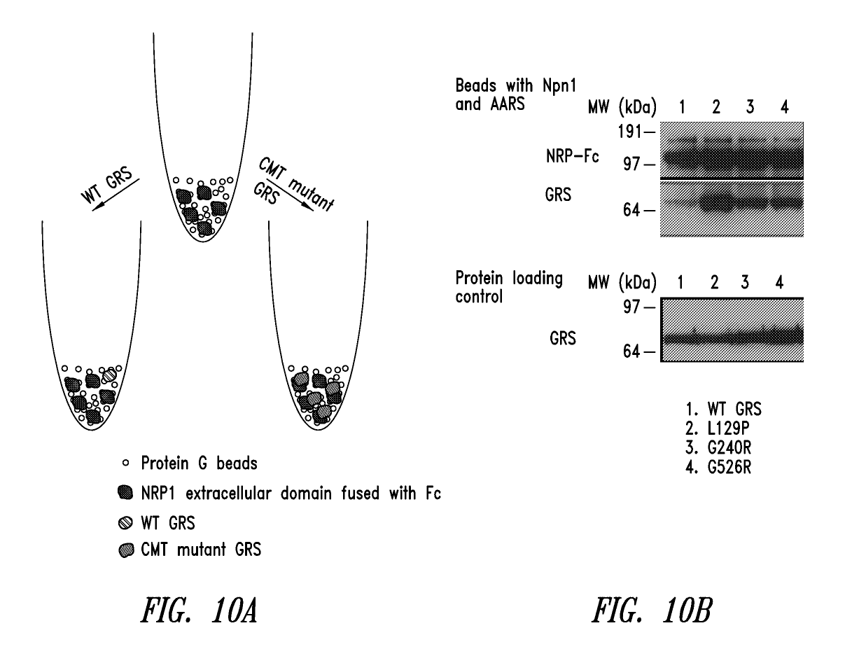

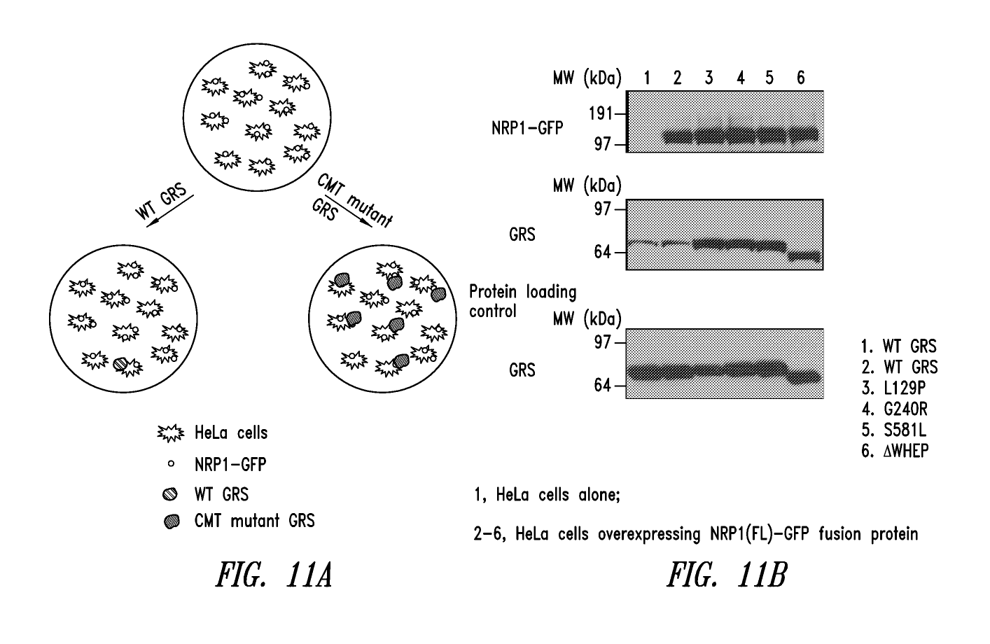

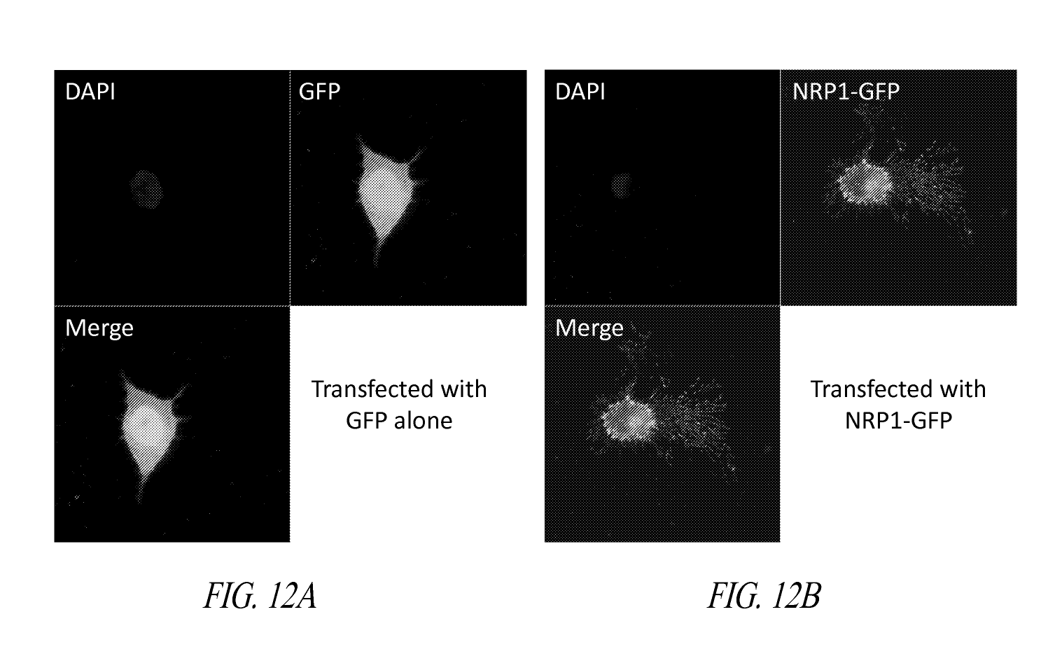

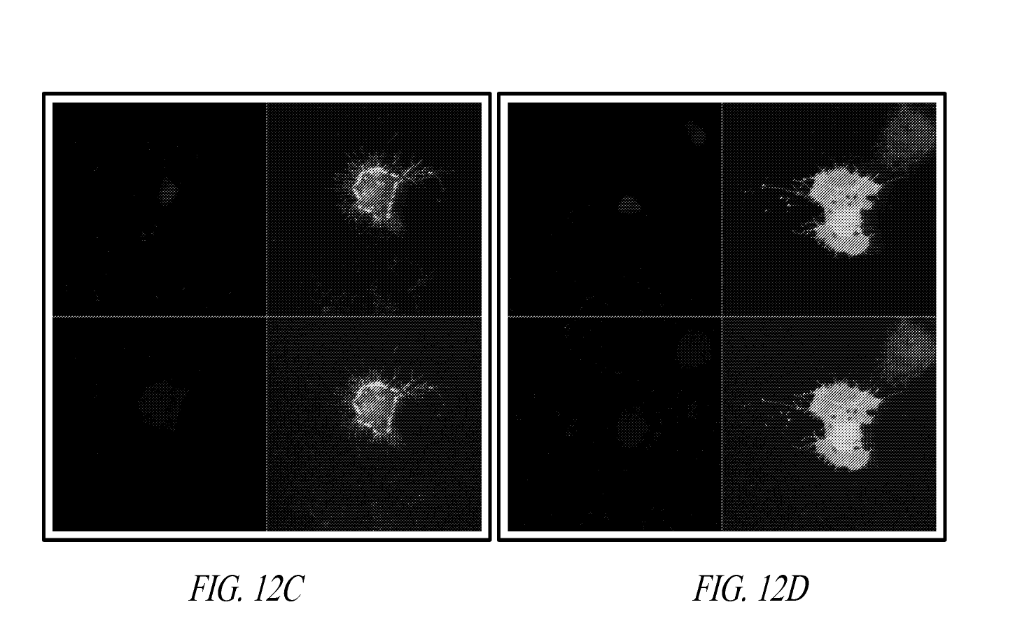

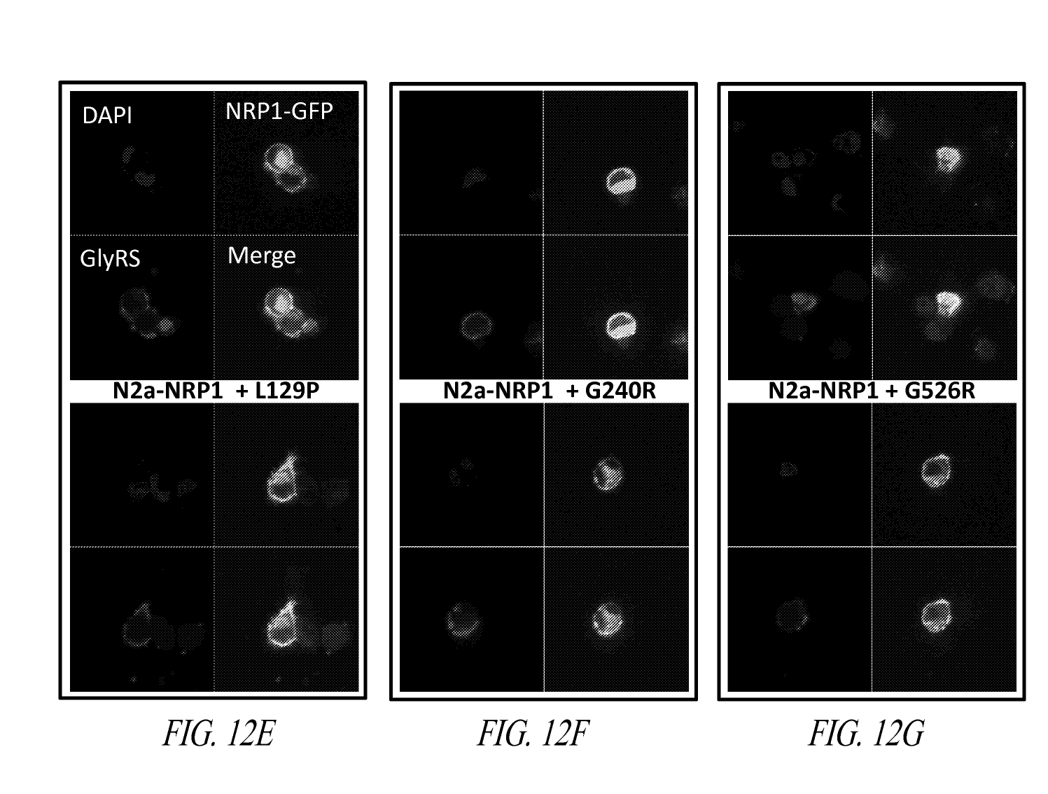

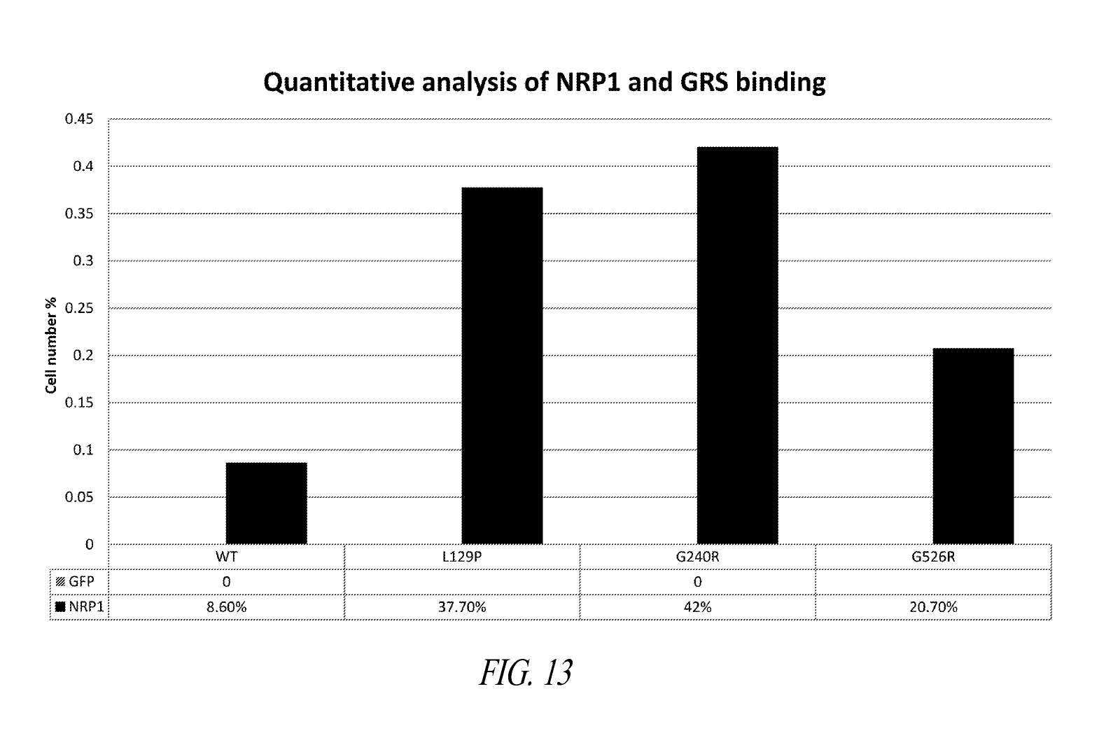

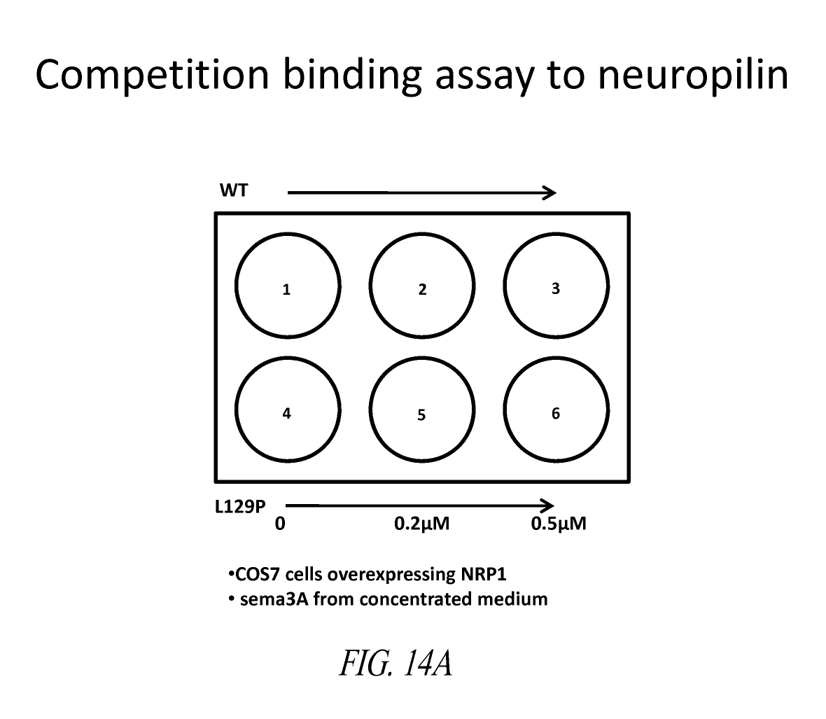

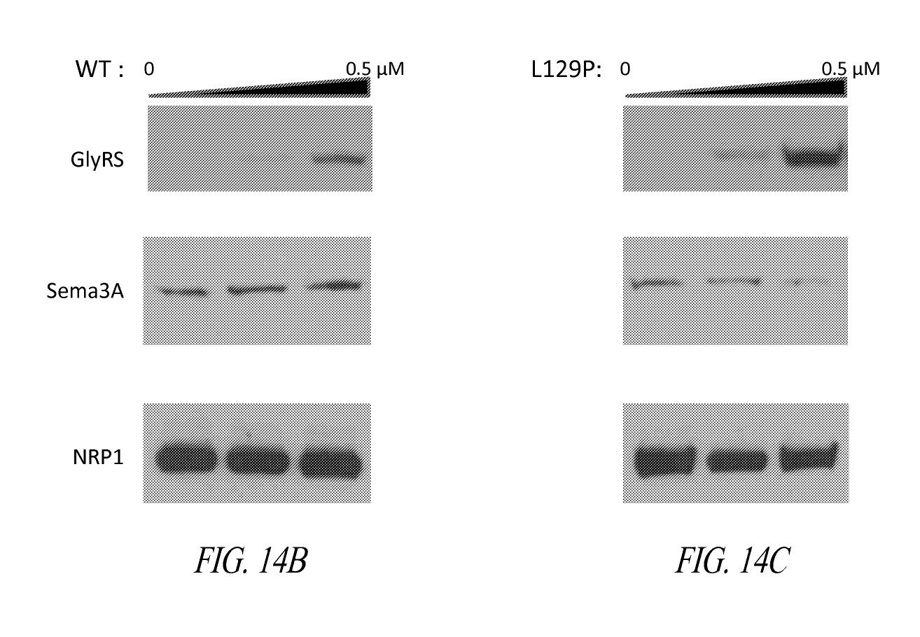

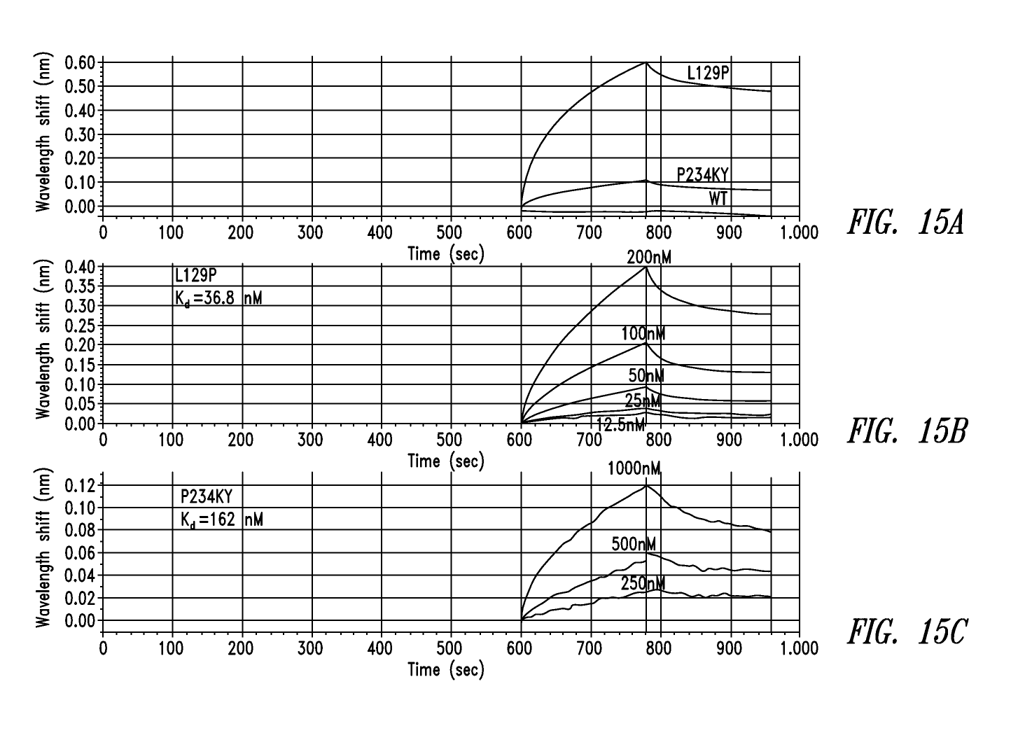

To identify the specific areas that are opened up by the mutations, the HDX MS results were mapped on the primary sequence and on the crystal structure of GlyRS (see FIG. 2 and FIG. 3). For dimer-weakening L129P and G240R mutants, large regions of GlyRS exhibited increased deuterium uptake compared to the WT protein and those areas include the dimerization interface (see FIG. 3A). The dimer interaction is mostly provided by three patches: F78-T137 (which includes the entire motif 1), F224-L242 and L252-E291 of the catalytic domain and is, to a lesser degree, contributed by S581-R606 of the anticodon binding domain). Peptides from these three patches exhibit some of the most pronounced increases in deuterium uptake (e.g., peptides F79-A83, M227-L257 and L258-R288 for L129P, and F79-A83, I232-N253 and L258-E279 for G240R GlyRS). In addition, other areas that are outside of the dimer interface also show large increases in deuterium uptake (e.g., peptides F147-K150 and E515-M531 for both L129P and G240R GlyRS) (see Table 1 and FIGS. 6A-6F). Hence, L129P and G240R mutations appear to induce conformational openings beyond those that dissociate the dimer.

For G526R and S581L mutations that promote dimer association, each mutant protein has one peptide that is less solvent exposed than its counterpart in the WT protein (see FIG. 2). Peptide I108-E123 (part of motif 1) in G526R and peptide R635-I645 in S581L GlyRS had lower deuterium incorporation and may be responsible for strengthening the dimer interactions of G526R and S581L GlyRS. In spite of these regions of reduced hydrogen-deuterium exchange, overall, both mutants are more solvent-exposed than is the WT protein (see FIG. 2 and FIGS. 6C and 6D). As for L129P and G240R GlyRS, this enhanced exposure includes most of, but is not limited to, the dimerization interface (see FIG. 3A). This pattern is also true for G598A GlyRS (see FIG. 2, FIG. 3A, and FIG. 6E).

Unexpectedly, the opened-up areas in each mutant protein largely overlap (see, e.g., FIG. 2). Eight consensus opened-up areas can be identified as ‘hot spots’. To qualify as a hot spot, in general, the area is well-covered in all the mutants tested, and each mutant peptide within the hot spot has more than 5% increase in HDX relative to the WT protein. Those hot spots include peptide A57-A83 adjacent to the WHEP domain and that partially overlaps with the dimer interface (Hot spot 1), L129-D161 that bridges motif 1 to Insertion I (Hot spot 2), N208-Y320 that covers a large area of the dimer interface and motif 2 (Hot spot 3), V366-H378 that is outside the dimer interface (Hot spot 4), P518-M531 that is part of motif 3 (Hot spot 5), L584-Y604 that is the site nearest to the other subunit in the anticodon binding domain (Hot spot 6), and F620-R635 and D654-A663 that are near the C-terminus and distal from the dimer interface (Hot spots 7 and 8). With the exception of motif 1, these hot spots are distributed over the entire dimer interface area, and also cover some areas outside the dimer interface.

At least two types of structural opening effects can be identified from these mutations. First is a local conformational change near the site of the mutation. Each CMT mutant protein tested has increased solvent accessibility in the vicinity of the substitution site (see FIG. 2). Second is a distal effect. As shown in Table 2 below, each mutation also increased the solvent accessibility near the other 12 known CMT-associated sites

Except for H418, all CMT-associated residues are covered by the HDX MS analysis of the WT and at least one mutant GlyRS. That all CMT-mutation-site-containing peptides in all mutants exhibit increased deuterium uptake suggests that any single CMT-causing mutation can induce a conformational opening that exposes all CMT-associated sites. Consistently, except for H418, D500, and S581, which were poorly covered in the HDX MS analysis, the other ten CMT-associated residues are all within the hot spots of the opened-up areas (see FIG. 2 and FIG. 3B.

SAXS analysis confirms the structure opening of G526R GlyRS in solution. Among the five mutants analyzed by AUC and HDX MS, particularly interesting are G526R and S581L, which facilitate dimerization, also show overall increased solvent accessibility, including to most of the dimerization interface. These mutants likely open up the structure without dissociating the dimer and, as a result, the thus likely expand the overall structure of the protein. To test that hypothesis, and to gain additional insight into the conformational change, SAXS analysis was used to compare one exemplary mutant G526R to WT GlyRS.

Scattering curves were measured for both G526R and WT GlyRS (see FIG. 4A). The pair distribution function p(r) obtained by an indirect Fourier transformation of the scattering curve showed that G526R GlyRS in solution is an elongated particle with a maximum dimension (Dₘₐₓ) of 185 Å, compared to Dₘₐₓ=160 Å for WT GlyRS (see FIG. 4B). The radius of gyration (R_{g}) evaluated by Guinier plots of the scattering curve yield R_{g}=44 Å (±0.4) for G526R, and R_{g}=41 Å (±0.9) for WT GlyRS. Both parameters (R_{g} and Dₘₐₓ) indicate that the mutant is larger in size than the WT protein, consistent with the HDX MS analysis and the above-noted hypothesis.

The crystal structure of GlyRS was then fitted onto the ab initio molecular envelopes of G526R and WT GlyRS generated from their scattering curves (see FIG. 4C). Extra unfitted density of each envelop presumably corresponds to the WHEP domain and/or insertion III that were disordered in the crystal structure. Because the extra density is located near the N-terminal residues in the crystal structure, it is most likely contributed from the WHEP domain. Consistently, a model of the WHEP domain with a helix-turn-helix structure fits well into the extra density (see FIG. 4C). The density of the molecular envelope of G526R and WT GlyRSs differs at the location of the WHEP domain suggesting a conformational change of the WHEP domain induced by the G526R mutation.

### Discussion

Several lines of evidence demonstrate that the GlyRS mutation-associated CMT phenotype is not simply caused by a deficiency in aminoacylation. Those results raised the possibility that CMT-causing mutations disrupt an unknown, peripheral neuron-specific function of GlyRS. On the one hand, certain tRNA synthetases are known to be multifunctional proteins (see Guo et al., Nat Rev Mol Cell Biol 11:668-674, 2010; and Guo et al., FEBS Lett 584:434-442, 2010). On the other hand, particularly because of the dominant nature of the GlyRS mutation-associated CMT phenotype, the disease may be linked to a gain-of-function pathogenic role only associated with the mutant GlyRS proteins. In that sense, the CMT-causing mutations in GlyRS resemble mutations in SOD1 that cause a severe motor neuron degenerative disease, amyotrophic lateral sclerosis (ALS). Mutant SOD1 proteins have gained toxic properties that promote aggregations in motor neurons, a common pathological feature for both familial and sporadic types of ALS (see Siddique et al., J Neural Transm Suppl 49:219-233, 1997; and Stathopulos et al., PNAS USA 100:7021-7026, 2003). However, overexpression of mutant GlyRS proteins in motor neurons did not promote aggregations (Antonellis et al., J Neurosci 26:10397-10406, 2006), and no aggregation of GlyRS or other misfolded proteins were found in CMT-mice expressing a mutant GlyRS (Stum et al., Mol Cell Neurosci 46:432-443, 2011). In view of the experiments described herein, these observations are consistent with the possibility that the gain of function of GlyRS CMT mutant proteins involve specific interaction(s) that lead to pathological consequences.

Also, in contrast to the well-spread ALS-causing mutations throughout the SOD1 structure, it has been confirmed herein that all CMT-causing mutations in GlyRS are located near the dimerization interface. Furthermore, HDX MS analysis elucidated a common area opened up by all tested mutations. These mutations have different effects on aminoacylation activity and on dimer stability, but the same neomorphic conformational opening. This opening may be a unifying feature of all CMT-causing mutations. The opened-up areas partially overlap with the dimerization interface and provide new surfaces for potential neomorphic interactions specific to the CMT mutants (see, e.g., FIG. 5). Unexpectedly, not only are the same neomorphic surfaces opened up by different CMT-causing mutations, but all CMT mutation-associated sites are also located within these surfaces. Thus, the mutations may strengthen a neomorphic interaction that contacts the opened-up areas, which would be potential drug targets for treating GlyRS mutation-linked CMT disease.

Considering the physiological concentration of tRNA synthetases being close to our measured K_{d} of dimer-monomer equilibrium for GlyRS (see Table 1), this synthetase is likely to exist in both dimer and monomer forms in vivo. The monomer forms would be especially predominant for L129P and G240R GlyRS and likely to be the form used for mediating neomorphic interaction(s) for those mutants. For other CMT-causing mutations that do not necessarily promote monomer formation, because the opened-up surface overlaps with most of the dimerization interface (see FIG. 3), the monomer forms of those mutants may also mediate neomorphic interaction(s) (see FIG. 5).

As discussed below (see Example 3), deletion of the WHEP domain from GlyRS induces a conformational change resembling that of the CMT-causing mutations (see FIG. 2 and FIG. 3). WHEP domains are also found in other human tRNA synthetases and are critically associated with the mechanisms used to expand and regulate a broad functionome of tRNA synthetases. For example, the WHEP domain of TrpRS regulates an angiostatic activity embedded in the synthetase (see Zhou et al., Nat Struct Mol Biol 17:57-61, 2010). Removal of the WHEP domain exposes a binding site for the VE-cadherin receptor that mediates the angiostatic effect of extracellular TrpRS. Possibly, the WHEP domain in GlyRS has a regulatory role like that in TrpRS, and the neomorphic structural opening is associated with an unknown physiological function of GlyRS that is suppressed by the WHEP domain. In this scenario, a CMT-causing mutation may simply disrupt the WHEP domain suppression and thereby give a gain-of-function phenotype. Moreover, the structural opening effect of the CMT mutations could be mediated through changing the WHEP domain conformation which, interestingly, is evident in our SAXS analysis with G526R GlyRS (see FIG. 4C). Therefore, whether the neomorphic structure opening is associated with a physiological or a pathological function, the gain of function of the CMT-causing mutants could due to the loss of function of the WHEP domain as a regulator.

Formation of the same neomorphic structural variant as a result of different CMT-causing mutations suggests that the ‘wild-type’ structure of GlyRS is on a ‘tipping point’ and is separated by a relatively small energy barrier from the neomorphic conformation. The studies described herein have not only demonstrated that spatially dispersed and seemingly unrelated mutations can perpetrate the same localized effect on a protein, but have also specifically defined the neomorphic regions that are “opened-up” by CMT-associated GlyRS mutations, regions that are otherwise hidden in WT GlyRS. These neomorphic regions represent strong candidate targets for drug discovery, diagnostic, and therapeutic uses related to neuronal disease such as CMT diseases.

### Example 2

### Two Newly Identified CMT-Associated Residues Localize to the Dimer Interface of Glycyl-tRNA Synthetase

Of the 13 CMT-linked mutations on GlyRS (from patients and mice) identified so far (see FIG. 1A), two—C201R from ENU-induced mice (corresponding to C157R in the human sequence) and P244L from a Japanese patient—have been recently identified (see, e.g., Abe and Hayasaka, J Hum Genet 54:310-312, 2009; and Achilli, et al., Dis Model Mech 2:359-373, 2009). Further to carrying out the conformational studies in solution, it was determined that the two newly identified CMT-associated residues P244 and C157, like the other 11, were localized near the dimer interface (see FIG. 1B). P244 is located on a hairpin structure (β8-β9) that forms an anti-parallel β-sheet across the dimer interface, and which harbors two other CMT-associated residues G240 and P234 (FIG. 1C). P234 and I280—other CMT-linked residues—have been reported to “kiss” across dimer interface (see Nangle et al., PNAS USA 104:11239-11244, 2007). Here, C157 is located adjacent to I280, and directly across from P234 of the other subunit. The three CMT-linked residues form a triad with a distance of ˜4 Å between each other (see FIG. 1C). Thus, 13 CMT-linked residues are all localized near the dimer interface.

### Example 3

CMT-Associated Structure Opening of Glycyl-tRNA Synthetase Mutants can be Regulated by the WHEP Domain

The metazoan-specific WHEP domain is dispensable for aminoacylation (Xie et al., supra). Because the CMT phenotype is not correlated with the aminoacylation activity of GlyRS, and because it was shown that the conformation of the WHEP domain appears primarily affected by one of the CMT mutations G526R (see FIG. 4C), it is possible that the WHEP domain plays a role in a CMT-associated mechanism and is involved in the structural opening induced by CMT mutations. The conformational consequence of removing the WHEP domain was therefore investigated.

According to the experiments described in Example 1, HDX MS analysis showed that the deletion of WHEP domain largely opens up GlyRS with average increase in deuterium uptake after 1 h exchange of 26% (see Table 1 and FIG. 6F). This level of increase is comparable with that of the CMT-causing mutants tested. Furthermore, the deletion mutant shares similar areas of structural opening, which include the dimerization interface (see FIG. 2 and FIG. 3A) and cover all CMT-associated mutation sites (Table 2). The similarity of the HDX MS results for WHEP-deleted GlyRS and for CMT-causing mutants suggests a role for the WHEP domain in suppressing a neomorphic conformational change of GlyRS that may lead to CMT.

### Example 4

### GlyRS is Secreted into Human and Mouse Serum and Secretion Requires ΔWHEP Domain

Experiments were performed to determine whether GlyRS can be found in human and mouse serum. Briefly, the level of GRS in serum samples from human patients and two mice was detected by immune-blotting with anti-GlyRS antibody. Cell necrosis was also monitored by detection of tubulin, GAPDH and LDH. N2a cell lysates were used as positive control for the antibodies. FIG. 9A shows that GlyRS antibodies were able to detect GlyRS protein in all of the human and mouse serum samples, showing that GlyRS is secreted from cells. About 2 μl serum was loaded in each lane.

Experiments were also performed to identify regions of GlyRS that associate with cell secretion of GlyRS. Wild-type GlyRS and ΔWHEP-GlyRS plasmids were prepared and transfected into COS7 cells using Lipofectamine 2000 (INVITROGEN®). After 24 hours, cell lysates and medium were collected. Total cell lysate and concentrated cell medium were then subjected to SDS/PAGE analysis. Cell necrosis was also monitored by GAPDH. GlyRS and GAPDH were detected by western blot using anti-V5 and anti-GAPDH antibodies, respectively. FIG. 9B shows that the WEEP domain of GlyRS is required for secretion of GlyRS. Full-length cytoplasmic GlyRS can be detected in both whole cell lysate and cell medium. The ΔWHEP mutant can be detected in whole cell lysate but not cell medium. These experiments thus show that at least a portion of the WHEP domain is required for cell secretion of GlyRS.

### Example 5

### CMT-Associated Mutations of GlyRS Show Enhanced Interaction with Neuropilin

In vitro pull-down and cell-surface binding assays were performed to assess the binding of wild-type GlyRS and CMT-associated GlyRS mutants to neuropilin. For the in vitro pull-down assay, recombinant neuropilin-1/Fc extracellular domain chimeric protein (R&D systems) was first immobilized to Protein G beads, and unbound NRP1 protein was washed away using DPBS buffer. Wild-type or CMT mutant GlyRS (L129P, G240R and G526R)-His fusion proteins were then added individually to the NRP1 immobilized beads and incubated for 1 hour at 4° C. Unbound GlyRS proteins were washed away with DPBS buffer, and SDS-loading buffer was added directly to the beads to elute the GlyRS-NRP1 interaction complexes, if any. NRP1 and GlyRS interaction were analyzed quantitatively by western blot using Anti-His antibody to detect the presence of GlyRS-His fusion proteins.

FIG. 10A shows the reaction scheme for the in vitro pull-down assay, and FIG. 10B shows the results of western-blotting with anti-His antibody (Lane 1 is wild-type GlyRS; Lane 2 is L129P mutant; Lane 3 is G240R mutant; and Lane 4 is G526R mutant). CMT-associated mutant GlyRS proteins (Lanes 2-4) show increased signal relative to wild-type GlyRS (Lane 1), demonstrating that these mutants interact more strongly with neuropilin than wild-type GlyRS.

For the cell surface binding assay, neuropilin (NRP1)-GFP plasmids were prepared and transfected into Hela cells using Lipofectamine 2000 (INVITROGEN®). After 24 hours, the transfected cells were washed with DPBS buffer and then incubated with 200 nM wild-type or CMT-associated GlyRS mutants (L129P, G240R, S581L, and ΔWHEP) in DPBS buffer for 1 hour at 4° C. Unbound GlyRS proteins were washed out with DPBS buffer, and SDS loading buffer was added directly in the wells to collect the whole cells, including the membrane fraction. Western blotting was then performed to detect His-GlyRS and GFP-NRP1 interactions.

FIG. 11A shows the experimental scheme of the cell-surface binding assay, and FIG. 11B shows the results of western-blotting to detect His-GRS and GFP-neuropilin (NRP1) interactions. Lane 1 shows Hela cells transfected only with wild-type GlyRS-His fusion protein (no GFP-NRP1), and Lane 2 shows Hela cells transfected with wild-type GlyRS-His fusion protein in combination with GFP-NRP1. Lanes 3-6 show Hela cells transfected with CMT-associated GlyRS mutants L129P (Lane 3), G240R (Lane 4), S581L (Lane 5), and ΔWHEP (Lane 6) in combination with GFP-NRP1. Increased detection of GlyRS-His fusions were observed in Lanes 4-6, showing that CMT-associated mutations to GlyRS enhance its interaction with neuropilin.

### Example 6

### CMT-Associated Mutations of GlyRS but not Wild-Type GlyRS Reverse Neuropilin-Induced Neurite Outgrowth

Confocal microscopy analysis was performed to assess the influence of GlyRS and CMT-associated GlyRS mutants on neuropilin-mediated neurite outgrowth. Mouse neuroblastoma N2a cells were transfected with either control pEGFP or NRP1-EGFP. After 24 hours, the cells were plated onto Fibronectin coated coverslips. Then N2a cells were then incubated with or without 200 nM wild-type GlyRS or CMT-associated GlyRS mutants (L129P, G240R, G526R) at 4° C. for 1 hour. Cells were then washed three times with DPBS buffer and fixed with 4% formaldehyde. The coverslips were mounted with Prolong Gold Antifade Reagent with DAPI (invitrogen) at 25° C. for overnight. Images were then taken using the Bio-Rad (Zeiss) Radiance 2100 Rainbow laser scanning confocal microscope (LSCM).

FIGS. 12A-12G show the results of these experiments. FIG. 12A shows GFP staining of N2a cells transfected with GFP alone, compared to FIG. 12B, which shows GFP staining of N2a cells transfected with GFP-NRP1. Increased neurite outgrowth can be observed in FIG. 12B, relative to FIG. 12A, showing that GFP-NP1 expression in N2a cells induces neurite outgrowth. FIGS. 12C and 12D show that incubation with wild-type GlyRS had no effect on neuropilin-induced neurite outgrowth. FIG. 12C shows the filopedia and FIG. 12D shows the lamellipodia of GFP-NRP1 expressing N2a cells incubated with wild-type GlyRS. FIGS. 12E (L129P mutant), 12F (G240R mutant), and 12G (G526R mutant) show that incubation with CMT-associated GlyRS mutants reverses neuropilin-induced neurite outgrowth, as evidenced by a relatively rounded cell morphology with little or no detectable neurite outgrowth. These images also show stronger staining of CMT-associated GlyRS mutants relative to wild-type GlyRS, suggesting that these GlyRS mutants interact more strongly with neuropilin-expressing cells than wild-type GlyRS.

FIG. 13 shows the results of a quantitative analysis of NRP1 and GlyRS binding or association, based on cell counts of confocal microscopy images of GlyRS (wild-type or mutants) and GFP-NRP1-expressing cells relative to GlyRS (wild-type or mutant) and GFP-only expressing cells. These results further support the interaction between CMT-associated GlyRS mutants and neuropilin.

### Example 7

### CMT-Associated Mutations of GlyRS but not Wild-Type GlyRS Competitively Inhibit Interaction Between Semaphorin-3A and Neuropilin

Competitive binding assays were performed to further characterize the binding of CMT-associated GlyRS mutants to neuropilin. Specifically, GlyRS proteins were tested for their ability to competitively inhibit the binding between neuropilin-1 and semaphorin-3A (sema3A), the latter being a specific receptor for neuropilin-1. Neuropilin (NRP1) plasmids were transfected into COS7 cells using Lipofectamine 2000 (INVITROGEN®). After 24 hours, the COS7 cells were washed with DPBS buffer and then incubated with sema3A (sema3A was collected from medium of COS7 cells overexpressing sema3A) and different concentration of wild-type GlyRS-His or CMT-associated GlyRS mutant L129P-His (at 0, 0.2 μM, and 0.5 μM) in DPBS buffer for 1 hour at 4° C. Unbound proteins were washed out with DPBS buffer, and SDS loading buffer was added directly to the wells to collect the whole cells including the membrane fraction. Western blotting was performed to detect the relative interaction of His-GRS or V5-sema3A with NRP1.

FIG. 14A illustrates the experimental scheme, and FIGS. 14B-14C show the results of western-blotting to detect interaction of His-tagged GlyRS or V5-tagged sema3A with NRP1. FIG. 14B shows the results for wild-type GlyRS, which does not competitively inhibit binding between sema3A and neuropilin, and FIG. 14C shows the results for the L129P mutant, which does competitively inhibit binding between sema3A and neuropilin. These results further demonstrate that CMT-associated GlyRS mutants specifically interact with neuropilin proteins, such as neuropilin-1.

### Example 8

### Quantitative Measurement of the Binding Affinity Between CMT-Associated GlyRS Mutants and Neuropilin

The binding constants between GlyRS CMT mutants (L129P and P234KY) and Neuropilin-1 (NRP1) were measured using Octet RED (ForteBio, Inc.). Octet RED utilizes bio-layer interferometry (BLI) technology that enables real-time analysis of protein-protein interactions on the surface of a fiber optic biosensor. The protein of interest is firstly immobilized on the biosensor and then exposed to the protein analyte in solution. The binding of two proteins changes the optical properties of the biosensor, leading to a shift in the wavelength of reflected light. This shift in wavelength allows the measurement of the determination of the binding constant.

Protein G-coated biosensors were first immobilized with 5 ug/mL NRP1-Fc chimeric protein (R&D systems) in 1× kinetics buffer (1×PBS, pH 7.4, 0.01% bovine serum albumin, and 0.002% Tween 20) at 30□C. The binding affinity experiments then included the following steps:

1. Baseline acquisition (˜60s);

2. NRP1 immobilization (˜300s);

3. Wash-out of unbound NRP1 (˜120s);

4. Second baseline acquisition (˜120s);

5. Association of GRS CMT mutants for measurement of kₒₙ (˜180s); and

6. Dissociation of GRS CMT mutants for measurement of k_{off} (˜180s).

Kinetic experiments were carried out by varying the concentration of GlyRS proteins flowing through the NRP1-immobilized biosensor. The dissociation constant of the GlyRS-NRP1 interaction was determined by k_{off}/kₒₙ. Global curve fittings used a 1:1 binding model.

As shown in FIG. 15A, no binding was detected between NRP-1 and WT GlyRS, but strong binding was detected between NRP-1 and each of the CMT-associated mutants L129P and P234KY. FIG. 15B shows the kinetic analysis of the binding between L129P and NRP-1 at five concentrations listed. Global fitting of the binding data for the five concentrations of L129P resulted in a dissociation constant of 36.8 nM. FIG. 15C shows the kinetic analysis of P234KY binding to NRP1 at three concentrations listed. Global fitting of binding data for the three concentrations of P234KY resulted in a dissociation constant of 162 nM.

### Example 9

### Generation of Phage Displayed Human Antibodies Selective to Mutant GlyRS1

To generate human monoclonal antibodies with selectivity for disease associated GlyRS mutants compared to the wild type GlyRS1 protein, phage displayed recombinant bivalent Fab mini antibodies (AbD Serotech, USA) are screened against full length human glycyl-tRNA synthetase (SEQ ID NO:1), comprising the L129P mutation, or against full length human glycyl-tRNA synthetase comprising the G240R mutation, or against full length human glycyl-tRNA synthetase comprising the S581L mutation, or against full length human glycyl-tRNA synthetase comprising the G526R mutation, or against full length human glycyl-tRNA synthetase comprising the G598A mutation, or against human glycyl-tRNA synthetase with a WHEP domain deletion, and then profiled against the full length wild type glycyl-tRNA synthetase (SEQ ID NO:1).

Antigen Production:

WT and mutant GlyRS are expressed and purified as described in the Materials and Methods section above.

Antibody Screening:

Phage Libraries (HUCAL®, Morphosys) are selected against recombinant GlyRS mutants, with three rounds of enrichment, following standard screening protocols (U.S. Pat. Nos. 6,300,064; 6,696,248; 6,706,484; 7,264,963). Antibodies displaying greater than a 5-fold ELISA signal for the mutant GlyRS antigen over background are selected for further evaluation.

Selected antibodies are screened in an ELISA format using the full length mutant GlyRS, as well as the full length wild type GlyRS (SEQ ID NO:1) proteins in parallel. Antibodies displaying selectivity for the mutant GlyRS compared to wild type GlyRS are selected for sequencing. Up to 20 positive antibodies are selected for sequencing to find unique clones. Up to 15 unique clones are expressed and purified, for subsequent analysis, as described below.

### Example 10

### Generation of Rabbit Monoclonal Antibodies Selective to Mutant GlyRS1

Generation of Polyclonal Antibodies:

To generate polyclonal antibodies with selectivity for a mutant GlyRS compared to the wild type GlyRS, rabbits are immunized with full length human glycyl-tRNA synthetase (SEQ ID NO:1) comprising the L129P mutation, or with full length human glycyl-tRNA synthetase comprising the G240R mutation, or with full length human glycyl-tRNA synthetase comprising the S581L mutation, or with full length human glycyl-tRNA synthetase comprising the G526R mutation, or with full length human glycyl-tRNA synthetase comprising the G598A mutation, or with human glycyl-tRNA synthetase with a WHEP domain deletion in complete Freunds adjuvant. Immunizations and test bleeds are conducted by Lampire Biological Laboratories (PA), or similar commercial vendor, using recombinant proteins prepared essentially as described above, and screened via ELISA assays.

Antigen Production:

WT and mutant GlyRS are expressed and purified as described in the Materials and Methods section above.

Antibody Screening:

Animals with plasma samples displaying greater than a 5-fold ELISA signal for the mutant GlyRS antigen over background are selected for further evaluation. To assess the relative binding of each polyclonal antibody to the mutant GlyRS compared to the wild type GlyRS, samples of each polyclonal antibody are incubated with equal amounts of either purified recombinant mutant GlyRS, or purified recombinant full length wild type GlyRS. Animals that are producing high affinity antibodies displaying selectivity for the mutant GlyRS compared to wild type GlyRS1 are selected for monoclonal antibody production as described below.

Monoclonal Antibody Production:

Once a suitable animal containing an antibody-producing cell has been identified or produced, spleen, lymph node or bone marrow tissue is removed, and a cell suspension of antibody-producing cells is prepared using standard techniques. See Harlow et al., (Antibodies: A Laboratory Manual, First Edition (1988) Cold Spring Harbor, N.Y.). Hybridomas are obtained by fusing such antibody producing cells with an immortal cell line. If rabbit-rabbit hybridomas are desired, the immortalized cell line will be from a rabbit. Such rabbit-derived fusion partners are known in the art and include, for example, cells of lymphoid origin, such as cells from a rabbit plasmacytoma as described in Spieker-Polet et al., PNAS USA. 92:9348-9352, 1995 and U.S. Pat. No. 5,675,063, rabbit derived 240E cells, or the TP-3 fusion partner described in U.S. Pat. No. 4,859,595, incorporated herein by reference in their entireties. If a rabbit-mouse hybridoma or a rat-mouse or mouse-mouse hybridoma, or the like, is desired, the mouse fusion partner will be derived from an immortalized cell line from a mouse, such as a cell of lymphoid origin, typically from a mouse myeloma cell line. A number of such cell lines are known in the art and are available from the ATCC. Examples of suitable immortal parental cells, include the mouse derived P3/X63-Ag8.653, P3/NS1/1-Ag4-1(NS-1), P3/X63Ag8.U1 (P3U1), SP2/O-Ag14 (Sp2/O, Sp2), PAI, F0, and BW5147; rat derived 210RCY3-Ag.2.3; human derived U-266AR1, GM1500-6TG-A1-2, UC729, CEM-AGR, DIR11, and CEM-T15; and chicken derived DT-40. For further descriptions of rabbit monoclonal antibodies and methods of making the same from rabbit-rabbit and rabbit-mouse fusions, see, e.g., U.S. Pat. No. 5,675,063 (rabbit-rabbit); U.S. Pat. No. 4,859,595 (rabbit-rabbit); U.S. Pat. No. 5,472,868 (rabbit-mouse); and U.S. Pat. No. 4,977,081 (rabbit-mouse). For a description of the production of conventional mouse monoclonal antibodies, see, for example, Kohler and Milstein, Nature (1975) 256:495-497.

Up to 20 positive antibodies clones are selected for sequencing and for subsequent analysis, as described below.

### Example 11

### Testing of Antibodies in Animal Models of CMT

Two mouse models of CMT2D that share pathological features with the human disease, with differing severity, have been described (Motley W W, et al. (2011). PLoS Genet 7(12): e1002399.doi:10.1371/journal.pgen.1002399) and are caused by dominant amino acid substitutions in GlyRS. The GlyRS^{Nmf249} allele (hereafter abbreviated Nmf249) causes reduced body weight and impaired mobility in heterozygous mice. Axon number and neuromuscular junction (NMJ) morphology are normal at post-natal day 7, but subsequently axons are lost without a reduction in myelin thickness; NMJs show partial and sometimes complete denervation (Sebum K L, et al. (2006) An active dominant mutation of glycyl-tRNA synthetase causes neuropathy in a Charcot-Marie-Tooth 2D mouse model. Neuron 51: 715-726). The Nmf249 allele is an insertion in the GlyRS gene that substitutes lysine and tyrosine for proline at position 278 in the mouse GlyRS protein, equivalent to a P234KY change in human GlyRS (Note: numbering differences are because the human annotation does not consider the N-terminal mitochondrial localization signal appended through alternative start codon usage).

The GlyRS^{C201R} allele (hereafter abbreviated C201R) is less severe and was identified in a chemical mutagenesis screen. In addition to impaired grip strength, these mice have impaired motor control, diminished muscle force, reduced weight, a shift towards smaller axon diameters, and some muscle denervation (Achilli F, et al., (2009) An ENU-induced mutation in mouse glycyl-tRNA synthetase (GARS) causes peripheral sensory and motor phenotypes creating a model of Charcot-Marie-Tooth type 2D peripheral neuropathy. Dis Model Mech 2: 359-373). The mouse C201R substitution is equivalent to C157R in human GARS. These two alleles demonstrate the spectrum of phenotypic severity in mice and provide a range of tests that can be used to quantify the effects on neuromuscular function.

To test the ability of antibodies to block the dominant negative mutation in GlyRS, candidate antibodies are prepared using standard procedures and prepared sterile at a concentration of about 10 mg/ml in PBS. CMT2D mice (about 30 days old) are injected sc with candidate antibodies in the range of 1 to 10 mg/kg on a weekly basis for 2 months. CMT2D mice (about 7 to 30 days old) are injected IP with candidate antibodies in the range of 1 to 10 mg/kg three times a week for 2 months. Control animals receive injections of PBS alone. Injected and control mice are monitored for behavioral changes, nerve conduction velocity changes and then the animals are euthanized by CO2 inhalation for tissue analysis as described below.

Tissue Lysate Preparation:

Spinal cord and sciatic nerve are isolated from animals immediately after they are euthanized by CO₂ inhalation. The tissues are frozen in liquid nitrogen and stored at −80° C. The tissues are then homogenized in 1% NP-40 in phosphate buffered saline (PBS) supplemented with Protease Inhibitor Cocktail Tablets (Roche, Basal, Switzerland) using a PowerGen Model 125 Homogenizer (Fisher Scientific, Pittsburgh, Pa.), centrifuged at 14,000 g for 10 min at 4° C. Cleared homogenates are then sonicated at 4° C. and centrifuged again at 14,000 g for 10 min. Protein concentrations are assessed using a Bradford assay (BioRad, Hurcules, Calif.). 20 μg of protein is then analyzed by immunoblot.

Teased Nerve Immunohistochemistry:

Teased nerve fibers are stained as described in the methods of Stum M G, et al., (2010) Mol Cell Neurosci 46: 432-443. In brief, sciatic nerves are excised, immediately placed in fresh 4% paraformaldehyde, and fixed on ice for 15 minutes. Nerves are then transferred to ice cold PBS for dissection and teasing. Using #5 forceps and 30 gauge needles, nerve sheaths are removed and the nerves are cut into 1 cm segments and teased apart at one end. The nerve segments are then transferred to a fresh Superfrost Plus Gold slides (Fisher Scientific, Pittsburgh, Pa.) and pulled from a drop of PBS onto a dry section of the slide to straighten the fibers for imaging. The slides are then dried overnight at room temperature and incubated in acetone at −20° C. for 10 min. The samples are then rehydrated with two 5 min incubations in PBS and blocked with 5% normal goat serum in PBS with 0.5% Triton X-100 for 1 h at room temperature. Primary antibodies rabbit anti-GARS (1:500) (Abeam), mouse anti-neurofilament with the 2H3 antibody (1:500) (Developmental Studies Hybridoma Bank, Iowa City, Iowa) are gently placed on the slide, covered with Parafilm coverslips (Pechinery Plastic, Chicago, Ill.) and stored in a humidified chamber overnight at 4° Celsius. After three 5-min washes in PBS, the samples are incubated in the following secondary antibodies diluted 1:1,000 in blocking solution: AlexaFluor 555 goat anti-rabbit, and AlexaFluor 488 goat anti-mouse IgG₁ (y1) (Invitrogen, Carlsbad, Calif.). The samples are covered with Parafilm coverslips and incubated for 2 h at room temperature. The samples are then washed three times in PBS for 5 min each in watch glasses and mounted with Vectashield (Vector Labs, Burlingame, Calif.).

Motor and Sensory Nerve Analysis:

The sensory and motor branches of the femoral nerve are isolated and fixed overnight in 2% glutaraldehyde and 2% paraformaldehyde in a 0.1 M cacodylate buffer. The tissue is then processed for transmission electron microscopy and embedded in plastic before 0.5 μm sections are cut and stained with toluidine blue. For axon counting and axon diameter measurement the images are captured using a Nikon Eclipse E600 microscope with 40× and 100× objectives. Axon counts are done using the Cell Counter Plug-in in ImageJ. Left and right nerves are averaged. Axon diameters are measured using the Measure and Label Plugin, also in ImageJ.

NMJ Imaging and Analysis:

Mouse plantaris muscles are surgically removed and fixed in freshly prepared 2% paraformaldehyde in PBS for four hours. The samples are then transferred to a blocking and permeabilizing solution of 5% normal goat serum and 0.5% Triton-X 100 in PBS for 1 h before they are pressed between two glass slides using a binder clip for 15 min, after which they are returned to the blocking and permeabilizing solution. The samples are then incubated overnight at 4° C. with 1:1,000 dilutions of anti-SV2 and anti-neurofilament (2H3) primary antibodies (Developmental Studies Hybridoma Bank, Iowa City, Iowa). After at least three 1 h washes in PBS with 0.5% Triton-X 100, the samples are transferred to blocking and permeabilizing solution with AlexaFluor 488 goat anti-mouse IgG₁ (y1) (Invitrogen, Carlsbad, Calif.) and α-bungarotoxin conjugated with Alexa Fluor 594. After incubation overnight at 4° C., the samples are washed three times for 1 h each and mounted with Vectashield mounting media (Vector Labs, Burlingame, Calif.) and imaged using a confocal microscope.

Confocal Microscopy:

Confocal images are gathered using a Carl Zeiss LSM 710 or Leica SP5 laser-scanning confocal microscope with a 63× objective. Z stacks are collapsed into projected images and merged using ImageJ. The color balance of the NMJ images is adjusted for clarity.

Nerve Conduction Studies:

Sciatic nerve conduction velocity is calculated by measuring the latency of compound motor action potentials recorded in the muscle of the left rear paw. The mice are anesthetized with 1% isofluorane and placed on a thermostatically regulated heating pad to maintain normal body temperature. Action potentials are produced by subcutaneous stimulation at the sciatic notch and at the ankle. For recording, the active needle electrode is inserted in the center of the paw and a reference electrode is placed in the skin between the first and second digits.

Antibodies showing efficacy in the animal models of CMT2D, are further evaluated for biophysical characteristics, affinity, stability and cross reactivity against the entire set of CMT disease associated mutations. Preferred antibodies showing optimal affinity and cross reactivity profiles are nominated as candidates for clinical studies.

1.-112. (canceled)

113. A method for blocking neuropilin activity, comprising contacting a neuropilin protein with a glycyl-tRNA synthetase (GlyRS) mutant polypeptide, wherein the GlyRS mutant polypeptide exhibits specific binding to the neuropilin protein.

114. The method of claim 113, where said GlyRS mutant competitively inhibits binding of the neuropilin protein to one or more neuropilin ligands.

115. The method of claim 114, where at least one of said one or more neuropilin ligands is a vascular endothelial growth factor (VEGF), a semaphorin, a placental growth factor, a heparin-binding protein, fibroblast growth factor-2, or a hepatocyte growth factor.

116. The method of claim 113, where said neuropilin activity is associated with one or more of angiogenesis, cell migration, cell invasion, cell metastasis, and cell adhesion.

117. The method of claim 113, wherein said GlyRS mutant is a mutant associated with Charcot-Marie-Tooth disease (CMT).

118. The method of claim 113, where said GlyRS mutant comprises one or more exposed neomorphic regions defined by amino acid residues A57-A83, L129-D161, N208-Y320, V366-H378, P518-M531, L584-Y604, F620-R635, and D654-A663 of the full-length human GlyRS, or a fragment thereof.

119. The method of claim 113, where said GlyRS mutant comprises one or more of A57V, E71G, L129P, C157R, P234KY, G240R, P244L, I280F, H418R, D500N, G526R, S581L, and G598A mutations relative to wild-type human GlyRS.

120. The method of claim 113, where said GlyRS mutant consists essentially of residues A57-A663 of wild-type human GlyRS, or an antigenic fragment thereof.

121. The method of claim 113, where said GlyRS mutant comprises amino acid residues F79-A83, M227-L257, I232-N253, L258-E279, F147-K150, E515-M531, A57-A663, A57-A83, L129-D161, N208-Y320, V366-H378, P518-531, L584-Y604, F620-R635, or D654-A663 of wild-type human GlyRS.

122. The method of claim 113, where said GlyRS mutant comprises L129P and G526R mutations relative to wild-type human GlyRS.

123. The method of claim 113, where the neuropilin protein is neuropilin-1 (NRP-1).

124. The method of claim 113, wherein contacting the neuropilin protein with the GlyRS mutant polypeptide comprises administering the GlyRS mutant polypeptide to a subject comprising the neuropilin protein.

125. The method of claim 124, wherein the subject has a disease or condition associated with one or more of increased neuropilin activity, increased neuropilin expression, increased activity of a neuropilin ligand, and increased expression of a neuropilin ligand.

126. The method of claim 125, where the subject has a cancer.

127. The method of claim 126, where the cancer is one or more of prostate cancer, breast cancer, colon cancer, rectal cancer, lung cancer, astrocytoma, ovarian cancer, testicular cancer, stomach cancer, bladder cancer, pancreatic cancer, liver cancer, kidney cancer, brain cancer, melanoma, non-melanoma skin cancer, bone cancer, lymphoma, leukemia, thyroid cancer, endometrial cancer, multiple myeloma, acute myeloid leukemia, neuroblastoma, glioblastoma, and non-Hodgkin's lymphoma.

128. A method for treating a neuronal disease, comprising administering to a subject in need thereof an inhibitor of a glycyl-tRNA synthetase (GlyRS) mutant polypeptide, wherein the GlyRS mutant competitively inhibits binding of the neuropilin protein to one or more neuropilin ligands.

129. The method of claim 128, wherein the GlyRS mutant polypeptide comprises one or more of A57V, E71G, L129P, C157R, P234KY, G240R, P244L, I280F, H418R, D500N, G526R, S581L, and G598A mutations.

130. The method of claim 129, wherein the inhibitor of the GlyRS mutant polypeptide is a monoclonal antibody specific for the GlyRS mutant polypeptide.

131. The method of claim 129, wherein the inhibitor of the GlyRS mutant polypeptide is a monoclonal antibody specific for the GlyRS mutant polypeptide comprising L129P and G526R mutations.

132. The method of claim 128, where the inhibitor of the GlyRS mutant polypeptide is an antibody or antigen-specific binding fragment for the GlyRS mutant polypeptide, or a small molecule that binds the GlyRS mutant polypeptide.

133. The method of claim 128, wherein the neuronal disease is a distal spinal muscular atrophy (dSMA) or a distal hereditary motor neuropathy (dHMN).

134. The method of claim 128, wherein the neuronal disease is Charcot-Marie-Tooth Disease Type 2D (CMT2D) or Distal Spinal Muscular Atrophy Type V (dSMA-V).
